(19) United States
(12) Patent Application Publication
(10) Pub. No.: US 2022/0235067 A1
(43) Pub. Date: Jul. 28, 2022

(54) COMPOSITIONS AND METHODS OF FABRICATION OF NEAR INFRARED DEVICES

(71) Applicant: The Regents of the University of California, Oakland, CA (US)

(72) Inventors: Guillermo C. Bazan, Santa Barbara, CA (US); Jaewon Lee, Goleta, CA (US); Seyeong Song, Santa Barbara, CA (US); Ziyue Zhu, Goleta, CA (US); Thuc-Quyen Nguyen, Santa Barbara, CA (US); Seo-Jin Ko, Goleta, CA (US)

(73) Assignee: The Regents of the University of California, Oakland, CA (US)

(21) Appl. No.: 17/617,777

(22) PCT Filed: Jun. 26, 2020
(86) PCT No.: PCT/US20/39963

§ 371 Date: Dec. 9, 2021

Related U.S. Application Data
(60) Provisional application No. 63/029,135, filed on May 22, 2020.
(60) Provisional application No. 62/965,620, filed on Jan. 24, 2020.
(60) Provisional application No. 62/866,849, filed on Jun. 26, 2019.

(51) Int. Cl. C07D 495/04; C07D 495/14; C07D 495/22; C07D 519/00; C07F 9/6568; C07F 7/30; C07F 7/08; H01L 51/00; H01L 51/42

(52) U.S. Cl. CPC C07D 495/04; H01L 51/0094; C07D 495/22; C07D 519/00; C07F 9/65686; C07F 7/30; C07F 7/0816; H01L 51/0043; H01L 51/0036; H01L 51/0051; H01L 51/0068; H01L 51/4246; H01L 51/0037; H01L 51/0071; H01L 51/0074; C07D 495/14

(57) ABSTRACT

A composition of matter including a donor including a dithiophene unit combined with a non-fullerene acceptor. Further disclosed is a device comprising an active region including the composition of matter. Example devices include a solar cell or a photodetector.

### CROSS REFERENCE TO RELATED APPLICATIONS

This application claims the benefit under 35 U.S.C. Section 119(e) of the following co-pending and commonly-assigned applications:

U.S. Provisional Patent Application No. 62/866,849, filed Jun. 26, 2019, by Guillermo C Bazan, Jaewon Lee, Seyeong Song, Thuc-Quyen Nguyen, and Seo-Jin Ko, entitled “COMPOSITIONS AND METHODS OF FABRICATION OF NEAR INFRARED PHOTOVOLTAIC DEVICES” Attorney's Docket No. 30794.735-US-P1 (2019-965-1);

U.S. Provisional Patent Application No. 63/029,135, filed May 22, 2020, by Guillermo C. Bazan, Jaewon Lee, Seyeong Song, Thuc-Quyen Nguyen, and Seo-Jin Ko, entitled “COMPOSITIONS AND METHODS OF FABRICATION OF NEAR INFRARED PHOTOVOLTAIC DEVICES” Attorney's Docket No. 30794.735-US-P2 (2019-965-2); and

U.S. Provisional Patent Application No. 62/965,620, filed Jan. 24, 2020, by Guillermo Bazan, Seyeong Song, Jaewon Lee, and Ziyue Zhu, entitled “NEAR INFRARED (NIR) ORGANIC ELECTRONIC DEVICES” Attorney's Docket No. 30794.760-US-P1 (2020-093-1);

all of which applications are incorporated by reference herein.

This application is related to the following co-pending an commonly assigned U.S. applications:

U.S. Utility patent application Ser. No. 16/179,294, filed Nov. 2, 2018, by Martin Seifrid, Guillermo C. Bazan, Jaewon Lee, Thuc-Quyen Nguyen, and Seo-Jin Ko, entitled “NARROW BANDGAP NON-FULLERENE ACCEPTORS AND DEVICES INCLUDING NARROW BANDGAP NON-FULLERENE ACCEPTORS,” Attorney's Docket No. 30794.657-US-U1 (2018-083), which application claims the benefit under 35 USC 119(e) of U.S. Provisional Patent Application No. 62/580,710, filed Nov. 2, 2017, by Martin Seifrid, Guillermo C. Bazan, Jaewon Lee, Thuc-Quyen Nguyen, and Seo-Jin Ko, entitled “NARROW BANDGAP NON-FULLERENE ACCEPTORS AND DEVICES INCLUDING NARROW BANDGAP NON-FULLERENE ACCEPTORS,” Attorney's Docket No. 30794.657-US-P1 (2018-083);

U.S. Utility patent application Ser. No. 16/792,000, filed Feb. 14, 2020, by Jaewon Lee, Seo-Jin Ko, Jianfei Huang, Martin Seifrid, Hengbin Wang, Thuc-Quyen Nguyen, and Guillermo C. Bazan, entitled “ORGANIC SOLAR CELL AND PHOTODETECTOR MATERIALS AND DEVICES” Attorney's Docket No. 30794.717-US-U1 (2019-400-1), which application claims the benefit under 35 USC 119(e) of U.S. Provisional Patent Application No. 62/806,232, filed Feb. 15, 2019, by Jaewon Lee, Seo-Jin Ko, Jianfei Huang, Martin Seifrid, Hengbin Wang, Thuc-Quyen Nguyen, and Guillermo C. Bazan, entitled “ORGANIC SOLAR CELL AND PHOTODETECTOR MATERIALS AND DEVICES” Attorney's Docket No. 30794.717-US-P1 (2019-400-1); and U.S. Provisional Patent Application No. 62/866,797, filed Jun. 26, 2019, by Thuc-Quyen Nguyen. Jianfei Huang. Jaewon Lee, Guillermo C Bazan, and Hengbin Wang, entitled “ORGANIC SOLAR CELL AND PHOTODETECTOR MATERIALS AND DEVICES” Attorney's Docket No. 30794.734-US-P1 (2019-937-1);

all of which applications are incorporated by reference herein.

### BACKGROUND OF THE INVENTION

### 1. Field of the Invention

The present invention relates to organic device (e.g., solar cells and photodetectors) and methods of making the same.

### 2. Description of the Related Art

(Note: This application references a number of different references as indicated throughout the specification by one or more reference numbers in superscripts, e.g., ^{[x]}. A list of these different publications ordered according to these reference numbers can be found below in the section entitled “References.” Each of these publications is incorporated by reference herein.

Organic optoelectronic devices have attracted attention for their inherent characteristics to be printed into ultra-thin, flexible, and conformal products through low-cost solution-processing techniques.^{[1,2]} Organic semiconductors offer clear advantages related to their molecular diversities of organic chromophores which allow organic photodiodes to be explored for a wide range of optical applications by tailoring the absorption spectra.^{[3-7]} Near-infrared (NIR) responsive organic semiconductors provide the potential in future applications such as semitransparent devices for building-integrated or green house systems.^{[8-10]} Photodetectors (OPDs) with NIR responsivity have plenty of applications such as image sensing, night surveillance, optical communication, and health monitoring.^{[11-13]}. NIR sensing has been conventionally realized with detectors based on single crystal inorganic semiconductor materials (e.g. Si, Ge, GaInAs, perovskite), which typically have drawbacks including costly processing, mechanical inflexibility, and sensitivity to temperature^{[37-39]}. Considering that the spectral response window of organic semiconductors can be readily tuned by rational molecular design, NIR OPDs have been emerged as a cost-effective material choice. Bulk-heterojunction (BHJ) structure consisting of a donor and an acceptor components to promote the charge separation are generally adopted in organic solar cells.^{[14-16]}

Conversely, the vast majority of state-of-the-art OPD systems comprise a semiconducting donor polymer governing the absorption range of the device, combined with a fullerene.^{[13]} These OPDs exhibit disadvantages over commercially available inorganic devices (e.g. their relatively low photoresponsivities in the NIR region), which can be attributed to the low external quantum efficiency (EQE) due to limited NIR light absorption of the fullerene based acceptors, poor carrier generation and extraction with increased charge recombination when the bandgap of the donor polymers become narrower, large noise current and consequently low detectivity related to the poorly suppressed charge transport in the dark under reverse bias. Narrow bandgap (NBG) non-fullerene electron acceptors (NFA) are an emerging class of NIR organic absorbers that overcome some shortages of the BHJ photodiodes based on the fullerenes. Of note are the structural flexibility that provides an opportunity to promote an energy level variability as well as to tailor absorption characteristics toward NIR light with outstanding optoelectronic responses.^{[17-19]} By combining narrow bandgap non-fullerene acceptors with wide gap donor polymers, strong absorption and high EQE across visible to NIR spectrum had led to solar cell efficiency up to 17%^{[40-41]}.

What is needed is organic devices with both improved efficiency at near infrared wavelengths and high transparency at visible wavelengths to realize efficient semitransparent and transparent NIR organic photodetectors and solar cells. The present disclosure satisfies this need.

### SUMMARY OF THE INVENTION

The present disclosure describes a surprising and unexpected combination of donors and Non-Fullerene Acceptors (NFAs) which have strong absorption in the near infrared NIR region but relatively small absorption (high transparency) in the visible light region. In one or more examples, the donors and NFAs have narrow bandgaps of less than 1.4 eV (e.g., in the range 1.1-1.4 eV) while absorbing wavelengths up to at least ˜1100 nm. Compositions with such donor and non-fullerene acceptors exhibit broader NIR absorption and high transparency at visible wavelengths detected by human eyesight, thereby opening up new applications for semitransparent and transparent organic electronic devices.

In a first example, a NIR polymer PM2 and small molecule X2 were selected as the donors, and various NIR NFAs were selected as the acceptors for the electronic devices. Surprisingly, solar cells with PCE (power conversion efficiency) over 9% and photo current over 15 mA/cm² were achieved. Surprisingly, photodetectors with responsivity as high as 0.36 A/W and specific detectivity close to 10¹² Jones in the near IR region (beyond 800 nm wavelength) were also achieved. Surprisingly, the devices also showed very high transparency in the visible light region.

In a second example, NIR absorbing donor (P2) and various NIR-non fullerene acceptors were synthesized and introduced for semitransparent organic solar cells and organic photodetectors. The P2 polymer has an ultra-narrow energy bandgap of ˜1.12 V and absorbs sunlight at wavelengths of up to ˜1200 nm. Compositions with such NIR non-fullerene acceptors also exhibited broader NIR light absorption and high transparency for wavelengths detectable by a human's naked eye. As a result, solar cells with PCE (power conversion efficiency) close to 3% and photo current over 10 mA/cm² were achieved. In one or more examples, the devices could surprisingly be fabricated without time consuming post-treatment.

Compositions of matter and devices according to embodiments described herein include, but are not limited to, the following.

1. A composition of matter, comprising:

a semiconducting compound of the structure (and isomers thereof):

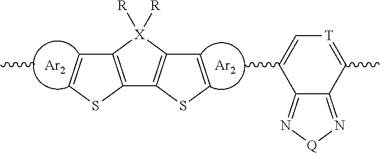

wherein:

each Ar₂ is independently a substituted or non-substituted aromatic functional group, or Ar₂ is nothing and the valence of the ring is completed with hydrogen;

each Q is independently O, S Se, or N—R₄;

each R and R₄ is independently hydrogen or a substituted or non-substituted alkyl, aryl, alkoxy or thioether chain;

T is N, C—F, or C—Cl;

X is C, Si, Ge, N or P; and

a non-fullerene acceptor combined with the semiconducting compound.

2. The composition of matter of example 1, wherein the semiconducting compound comprises the structure:

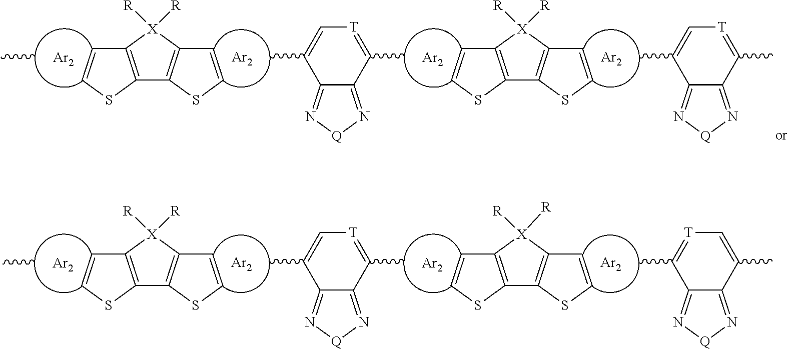

3. The composition of matter of example 2, wherein the semiconducting compound comprises the structure:

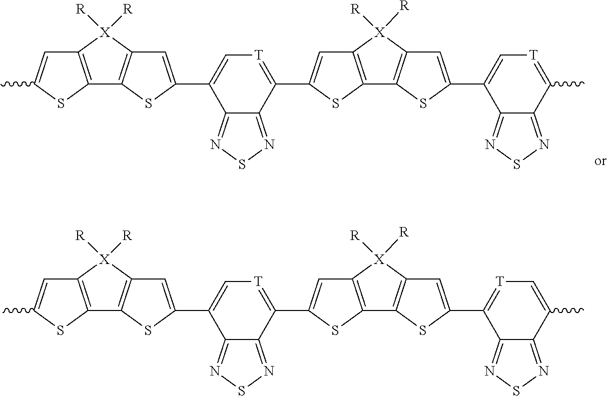

4. The composition of matter of example 1, wherein the semiconducting compound comprises the structure:

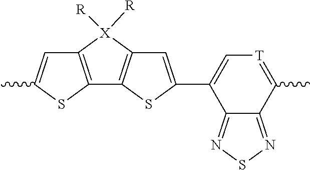

wherein:

each R is independently hydrogen or a substituted or non-substituted alkyl, aryl, alkoxy or thioether chain;

T is N, C—F, or C—Cl; and

X is C, Si, Ge, N or P.

5. The composition of matter of example 1, wherein the semiconducting compound further comprises the structure:

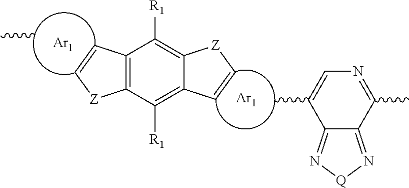

wherein:

each Ar₁ is independently a substituted or non-substituted aromatic functional group, or each Ar₁ is nothing and the valence of the ring is completed with hydrogen.

each Z is independently O, S, Se, or N—R₄;

each R₁, R₄ is independently hydrogen or a substituted or non-substituted alkyl, aryl, alkoxy or thioether chain.

each Q is independently O, S Se, or N—R₄;

6. The composition of matter of example 5, wherein the semiconducting compound comprises the structure:

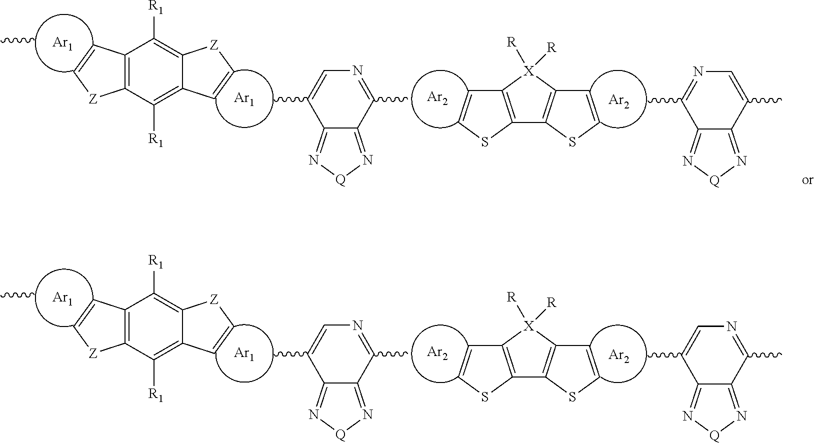

7. The composition of matter of example 6, wherein the semiconducting compound comprises the structure:

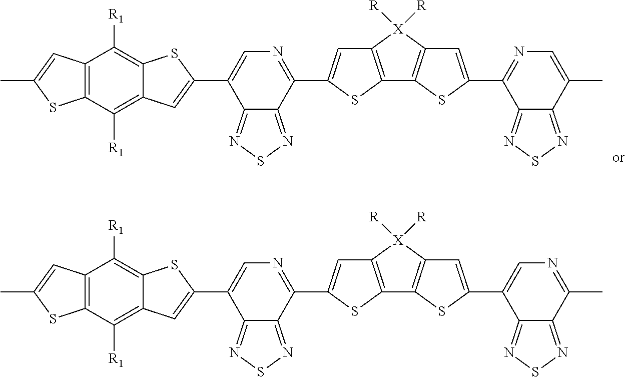

8. The composition of matter of any of the examples 1-7, wherein the non-fullerene acceptor has the structure:

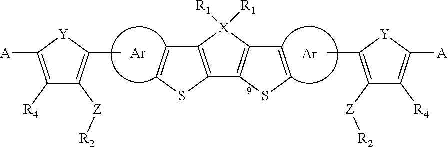

wherein:

each Ar is independently a substituted or non-substituted aromatic functional group, or each Ar is independently nothing and the valence of its respective thiophene ring is completed with hydrogen;

X is C, Si, Ge, N or P; Y is O, S, Se or N—R₃;

Z is O, S, Se, or N—R₃;

A is an acceptor moiety; and

each R₁, R₂ and R₃ is independently hydrogen or a substituted or non-substituted alkyl, aryl or alkoxy chain; R₄ is either a hydrogen or the same as Z—R₂.

9. The composition of matter of any of the examples 1-8, wherein the non-fullerene acceptor has a bandgap less than or equal to the bandgap of the semiconducting compound.

10. The composition of matter of any of the examples 1-9, wherein the semiconducting compound comprises a semiconducting polymer having a repeat unit comprising the structure of any of the examples 1-7.

11. The composition of matter of example 10, wherein the semiconducting polymer comprises a semiconducting polymer having the structure:

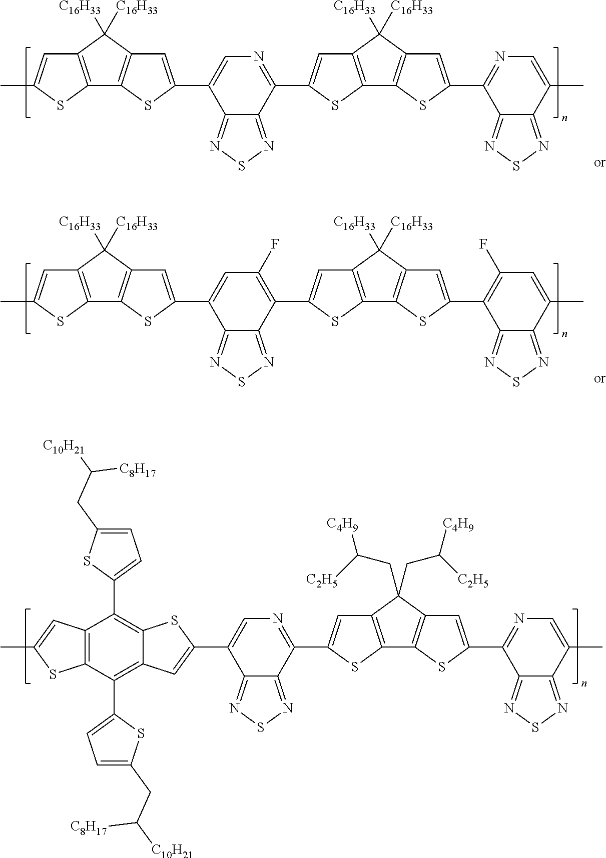

12. The composition of matter of examples 10 or 11, wherein the acceptor unit in the semiconducting compound is regioregularly arranged along the conjugated main chain section (the side chains comprising C and H, e.g., C₁₀H₂₁ can be any R comprising a substituted or non-substituted alkyl, aryl or alkoxy chain as described herein.

13. The composition of matter of any of the examples 1-12, wherein the non-fullerene acceptor comprises:

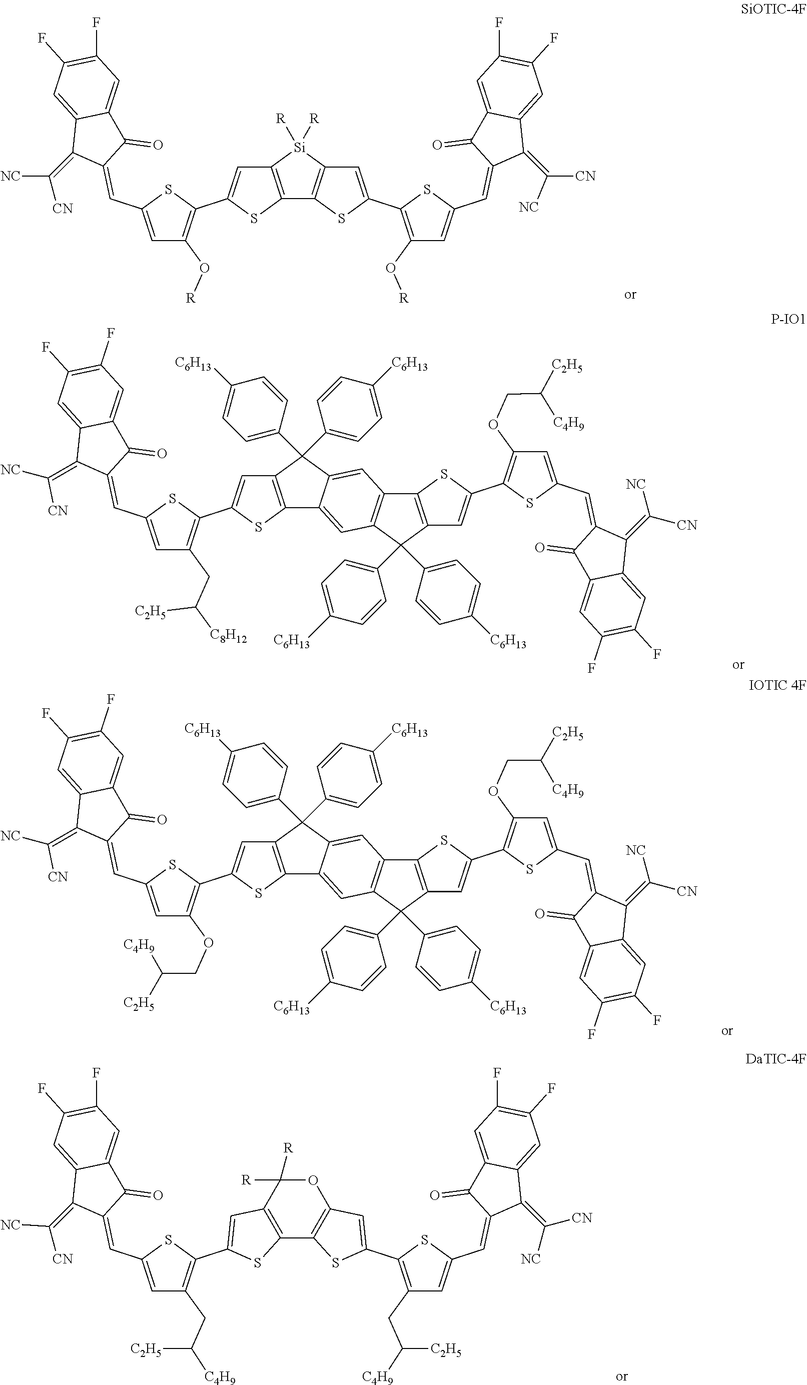

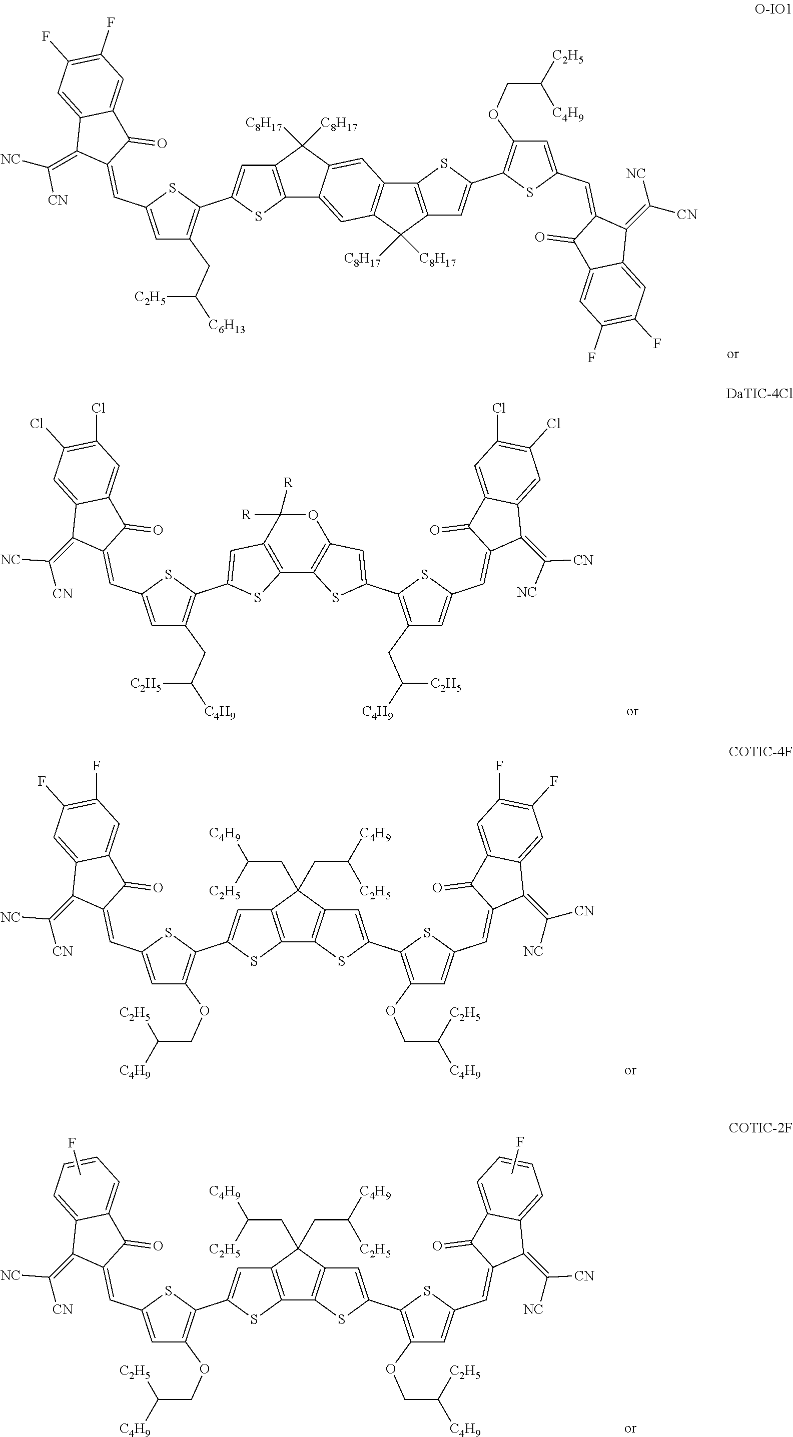

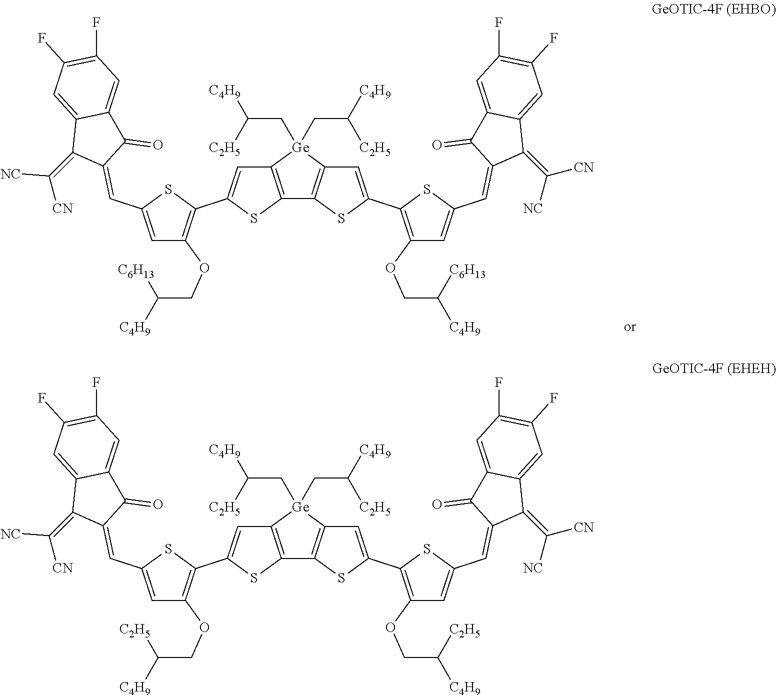

wherein R is a solubilizing chain comprising a substituted or non-substituted alkyl, aryl or alkoxy chain and the side chains comprising C and H may be any solubilizing chain comprising a substituted or non-substituted alkyl, aryl or alkoxy chain.

14. The composition of matter of any of the examples 1-13, wherein the non-fullerene acceptor has the structure:

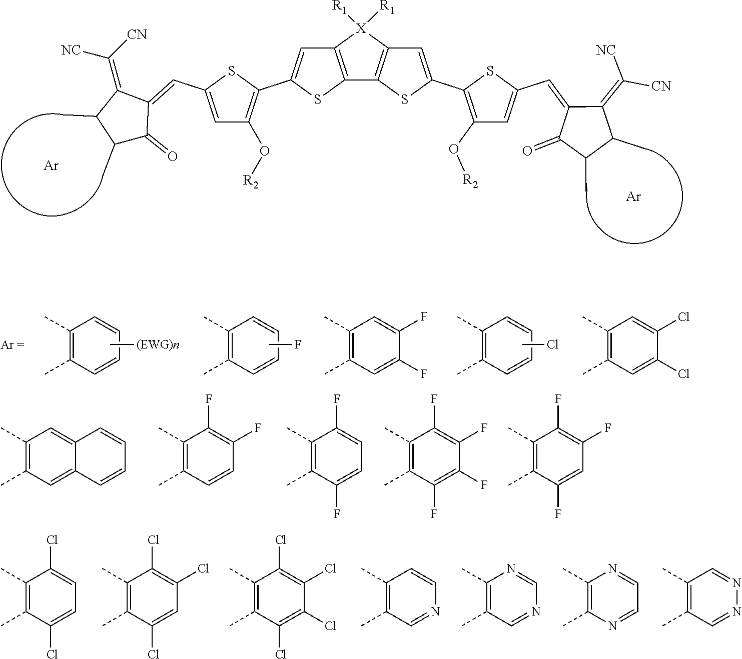

wherein n is an integer; X is C, Si, Ge, N; EWG=any electron withdrawing group; and each R₁ and R₂ is independently hydrogen or a substituted or non-substituted alkyl, aryl or alkoxy chain.

15. The composition of matter of any of the examples 1-14, wherein the semiconducting compound comprises a semiconducting small molecule having a repeat unit comprising the structure of any of the examples 1-7.

16. The composition of matter of example 15, wherein the semiconducting compound is a small molecule donor comprising the structure of E-A-(D1-A)ₙ-E, wherein:

D1 is

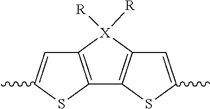

A is

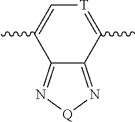

each R is independently hydrogen or a substituted or non-substituted alkyl, aryl, alkoxy or thioether chain;

X is C, Si, Ge, N or P;

each T is independently C—H, N, C—F or C—Cl;

Q is O, S, Se or N—R₄,

E is an alkylated bithiophene, and

n=1, 2 or 3.

16a. The composition of matter of any of the examples 1-6, wherein the semiconducting compound has a bandgap less than or equal to the bandgap of the non-fullerene acceptor.

17. The composition of matter of any of the examples 1-16, further comprising a bulk heterojunction comprising the composition of matter, wherein the semiconducting compound comprises a donor forming an interconnected network and heterointerface with the non fullerene acceptor, the donor and the acceptor are phase separated, and the donor phase is optionally crystalline.

18. The composition of matter of any of the examples 1-17, wherein the non fullerene acceptor has a bandgap of 1.3 eV or less (e.g., in a range of 0.8 eV-1.3 eV or in a range of 1 eV-1.3 eV where eV is electron volts.

19. A device comprising the composition of matter of any of the examples 1-18, wherein the device comprises a solar cell.

20. A device comprising the composition of matter of any of the examples 1-19, wherein the device comprises a photodetector.

21. The device of any of the examples 19-20, further comprising an active region comprising the composition of matter of any of the examples 1-18, wherein holes and electrons are generated in the active region in response to electromagnetic radiation incident on the active region,

the electrons are collected in the non-fullerene acceptor and are transmitted through to a cathode,

the holes are collected in the semiconducting compound comprising a donor and transmitted through to an anode,

so that the device outputs current in response to the electromagnetic radiation.

22. The device of any of the examples 19-21, comprising:

a film comprising a thickness of less than 1 micrometer and comprising:
the semiconducting compound having a transmittance of at least 70% for visible electromagnetic radiation having the wavelength in a range of 400 nm to 600 nm, and
the photodetector having an external quantum efficiency (EQE) of at least 30%, a responsivity of at least 0.1 A/W (amps per watt), and a specific detectivity of at least 10¹⁰ Jones for the electromagnetic radiation having the wavelength in a range of 700 nm-900 nm.

23. The device of example 22, wherein the composition of matter has the structure:

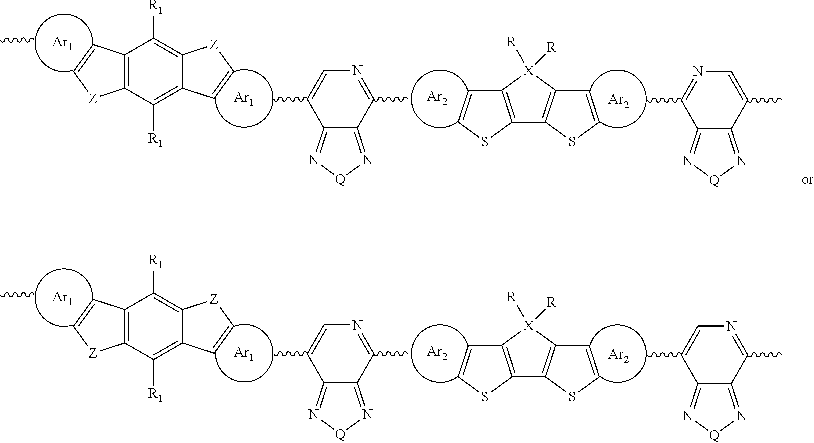

each Ar₁ and Ar₂ is independently a substituted or non-substituted aromatic functional group, or each Ar₁ and Ar₂ is nothing and the valence of the ring is completed with hydrogen.

each Z is independently O, S, Se, or N—R₄;

each X is C, Si, Ge, N or P;

each Q is independently O, S Se, or N—R₄.

and

each R, R₁ and R₄ is independently hydrogen or a substituted or non-substituted alkyl, aryl, alkoxy or thioether chain.

24. The device of example 23, wherein the semiconducting compound has the structure:

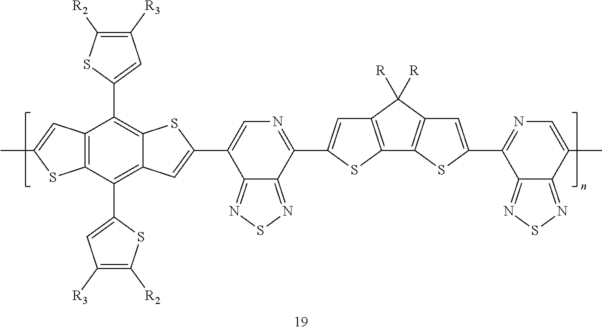

where each R, R₂ and R₃ is independently hydrogen or a substituted or non-substituted alkyl, aryl, alkoxy or thioether chain, and

n is an integer.

25. The device of example 22, further comprising:

a source of the visible electromagnetic radiation;

the photodetector on the source; and

a cover or window on the photodetector, such that the visible electromagnetic radiation is transmitted through the photodetector and the cover or window to a viewer.

26. The device of any of the examples 20-25, further comprising a biomedical sensor, wherein the photodetector measures electromagnetic radiation scattered or reflected from living tissue or cells.

27. A device, comprising:

an active region comprising organic semiconducting compounds (e.g., of any of the examples 1-18) outputting an electrical signal in response to electromagnetic radiation incident on the active region, the active region having:

a thickness less than 1 micrometer;

a transmittivity of at least 70% for the electromagnetic radiation having the wavelength in a range of 400 nanometers (nm) to 600 nm, and

an EQE of at least 30% for the electromagnetic radiation having the wavelength in a range of 700 nm-900 nm.

28. The device of example 27, wherein the semiconducting compound has the structure:

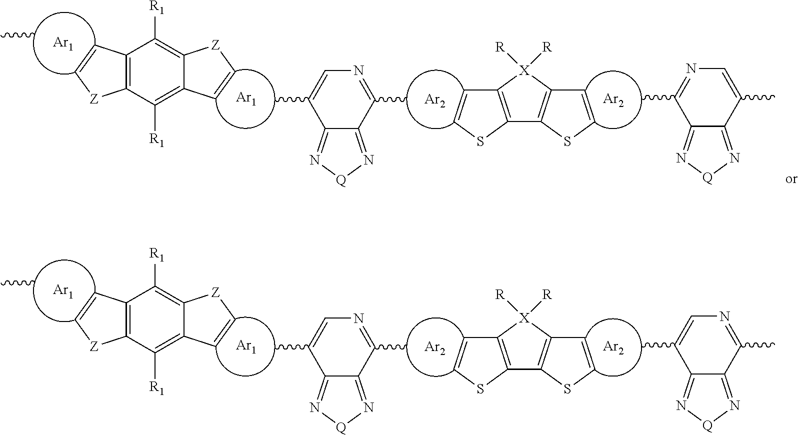

each Ar₁ and Ar₂ is independently a substituted or non-substituted aromatic functional group, or each Ar₁ and Ar₂ is nothing and the valence of the ring is completed with hydrogen.

each Z is independently O, S, Se, or N—R₄;

each X is C, Si, Ge, N or P;

each Q is independently O, S Se, or N—R₄

and

each R, R₁ and R₄ is independently hydrogen or a substituted or non-substituted alkyl, aryl, alkoxy or thioether chain.

29. An infrared photodetector, comprising:

a first electrode;

a first carrier transport layer;

an active layer, wherein the first carrier transport layer is between the first electrode and the active layer;

a second carrier transport layer, wherein the active layer is between the first carrier transport layer and the second carrier transport layer; and

a second electrode on the second carrier transport layer, wherein:

the active layer comprises the composition of matter of any of the examples 1-18, and

a transmittance of the photodetector is 50% or more at the wavelengths of 400-600 nm.

30. The photodetector of example 29, wherein the semiconducting compound in the active layer comprises P2.

31. The photodetector of example 29, wherein the semiconducting compound in the active layer comprises PM2.

32. A device comprising the photodetector of any of the examples 29-31, comprising:

a display emitting the wavelengths;

the photodetector on or above the display;

a screen on or above the photodetector, wherein the display is readable by eye of a viewer through the photodetector; and

a circuit connected to the photodetector, the circuit determining a gesture of the viewer from a signal outputted from photodetector in response to infrared radiation incident on the photodetector.

33. The device of example 32, wherein the photodetector has a thickness of 3 micrometers or less.

### BRIEF DESCRIPTION OF THE DRAWINGS

Referring now to the drawings in which like reference numbers represent corresponding parts throughout:

FIG. 1. (a) Chemical structure of X2, PM2, ITIC-4F, IOTIC-4F and SiOTIC-4F. (b) Energy level diagram of the materials.

FIG. 2. (a) Film absorption spectra of X2 and PM2 and (b) film absorption spectra of ITIC-4F, IOTIC-4F and SiOTIC-4F.

FIG. 3. (a) J-V characteristics and (b) EQE spectra of the OPV devices blended of X2:ITIC-4F, PM2:IOTIC-4F and PM2:SiOTIC-4F under illumination of an AM 1.5 G at 100 mW cm⁻².

FIGS. 4(a)-(h). Device performance of blends comprising X2 with different NFAs and as a function of processing conditions, showing (a) current density v. voltage for X2:IOTIC-4F, (b) table of device performance for IOTIC-4F, (c) current density v. voltage for X2:IOTIC-2F, (d) table of device performance for IOTIC-2F, (e) current density v. voltage for X2:ITIC-4F, (f) table of device performance for ITIC-4F, (g) current density v. voltage for various X2:NFA, (h) table of device performance for various X2:NFA.

FIG. 4(i). Summary of device performances for solar cell devices comprising X2:NFA blends having the device structure: ITO/PEDOT:PSS/Active/ZnO NPs/Al; or ITO/ZnO/Active/MoOx/Ag.

FIGS. 5(a)-(d). Absorption spectra of NIR NFAs and PM2:NFA blends, for (a) PM2, SiOTIC-4F, IOTIC-4F, CITC-4F, (b) blends of PM2:DaTIC-4X (X=H, F, Cl), (c) CTIC-4F, SiOTIC-4F, IOTIC-4F, CETIC, O-IO1, P-IO1.

FIGS. 6(a)-(e). Film transmittance of NIR PM2:NFA devices, for (a) PM2:IOTIC-4F, (b) PM2:CTIC-4F, and (c) PM2:SiOTIC-4F, (d) PM2:DaTIC-4X (X=H, F, Cl), and (e) PM2:IOTIC-4F and PM2:CTIC-4F.

FIG. 7(a)-(i). J-V characteristics and EQE spectra of PM2:NFA solar cell devices. Device structure: ITO/PEDOT:PSS/Active/ZnO NPs/Al; or ITO/ZnO/Active/MoOx/Ag, showing (a) current density v voltage for PM2:ITIC-Th device, (b) current density v voltage for PM2:SiOTIC-4F device, (c) current density v. voltage for PM2:IOTIC-4F device, (d) EQE for PM2:IOTIC-4F device, (e) EQE for PM2:SiOTIC-4F device, (f) atomic force microscope image showing the morphology of 10 PM2:SiOTIC-4F blend, showing surface height profile (brighter is higher) (g) Current density for PM2:CTIC-4F device, (h) EQE for PM2:CTIC-4F device, (i) current density for PM2:DaTIC-4X (X=H, F, Cl) device, (j) EQE for PM2:DaTIC-4X (X=H, F, Cl) device, (k) current density v. voltage PM2:O-IO1 (O-IO1-4F) device, (l) current density PM2:p-IO1 (p-IO1-4F) device, and (m) EQE PM2:O-IO1 device.

FIG. 8. Summary of device performances for solar cell devices comprising PM2:NFA blends having the device structure: ITO/PEDOT:PSS/Active/ZnO NPs/Al; or ITO/ZnO/Active/MoOx/Ag.

FIGS. 9(a)-(c). Tables showing device performance of PM2 devices as a function of processing conditions (a) PM2:SiOTIC-4F devices, (b) PM2:IOTIC-4F devices (c) other PM2 devices.

FIGS. 10(a)-(d). Device performance (a. EQE; b. dark current, c. responsivity and d. specific detectivity) of NIR PM2:IOTIC-4F and PM2:CTIC-4F photodetectors.

FIGS. 10(e)-(g) OPD device performance including responsivity (e) specific detectivity (f) and (g) tabulated data for PM2:SiOTIC-4F photodetectors.

FIGS. 10(h)-(i) OPD device performance, including (h) current density, (i) transmittance for PM2:SiOTIC.

FIGS. 10(j)-(l). Device performance of j. EQE; k. dark current, 1. transmittance of NIR X2:ITIC-4F photodetectors.

FIGS. 10(m)-(o). Device performance of (m) responsivity, (n) specific detectivity and (o) table summary of NIR X2:ITIC-4F photodetectors.

FIG. 11. Summary of PM2:IOTIC-4F and PM2:CTIC-4F NIR photodetector device performances.

FIG. 12 (a) Chemical structure of P2, COTIC-2F, CO6IC, C2P1, GeOTIC-4F-EH and GeOTIC-4F-BO.

FIG. 13 (a) Absorption spectra of P2 and (b) Absorption spectra of COTIC-2F, CO6IC, C2P1, GeOTIC-4F-EH and GeOTIC-4F-BO in solution.

FIG. 13 (c) Transmittance, (d, e) J-V characteristics and (f) EQE spectra of P2:NFA solar cell devices.

FIGS. 13(g)-(k) OPD device performance including (g) dark current (h) responsivity (j) specific detectivity (k) tabulated data for P2:IOTIC-4F, P2:o-IO1, and P2:COTIC-4F photodetectors.

FIG. 14(a) Device (e.g., solar cell or photodetector device) structure.

FIG. 14(b) Device on a window or display.

FIG. 15(a). Summary of device performances for solar cell devices comprising P2:NFA blends having the device structure: ITO/PEDOT:PSS/Active/ZnO NPs/Al; or ITO/ZnO/Active/MoOx/Ag.

FIG. 15(b). Further examples of NFAs.

FIG. 16. Example display application of photodetectors according to examples described herein.

FIG. 17(a)-(b). Further example applications of photodetectors according to examples described herein, wherein (a) illustrates a biomedical sensor and (b) illustrates a metrology device.

FIG. 17(c). Schematic of a bulk heterojunction comprising donor domains and NFA domains in the active region between a first electrode and a second electrode of a device according to one or more examples described herein.

FIG. 18. Flowchart illustrating a method of making a device or composition of matter.

### DETAILED DESCRIPTION OF THE INVENTION

In the following description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

### TECHNICAL DESCRIPTION

### A. First Example: Non Fullerene Acceptor (NFA) Combined with X2 and PM2 Donors

Small molecule organic solar cells exhibit many advantageous properties including, but not limited to, the possibility of tailoring their orbitals and molecular structure to achieve strong absorption, good reproducibility of synthesis, and high crystallinity for efficient charge transport.

Small molecule (SM) donor X2 and polymer donor PM2 are narrow band gap donors with optical bandgap (E_{g}^{opt}) of 1.41 eV and 1.42 eV respectively^{[14-19]} and exhibiting absorption up to ˜ 900 nm in near IR region. The optimum BHJ morphology of OSCs (organic solar cells) requires nanoscale intermixed domains of donor and acceptor components with a bicontinuous network for efficient exciton splitting and charge transport^{[20]}. Solution-processed polymer solar cells have been well established in the past decade^{[21]}. However, in comparison with polymer-based OSCs, the morphological properties and the consequent photovoltaic properties of SM-OSCs are more sensitive to the conditions during film-forming process^{[22]}. Crystallization of molecular donors is often crucial to ensure intermolecular interaction and phase separation required for the efficient photocurrent generation.^{[5-7,22]} Thus, additional processing methods, i.e. solvent additives, thermal annealing (TA), and/or solvent vapor annealing (SVA) are required to kinetically favored crystallization and phase separation^{[23-26]}. On the other hand, donor materials, such as X2, with a strong ability to crystallize without requiring additional treatments can produce favorable inter-connected networks and phase separation from the blends with fullerene.^{[27]} Such compositions with non-fullerene small molecules as acceptor may yield highly efficient NIR organic solar cells via continuous, high throughput, and environmentally friendly manufacturing routes.

A1. Optical and Electrical Property of NIR Materials According to the First Example

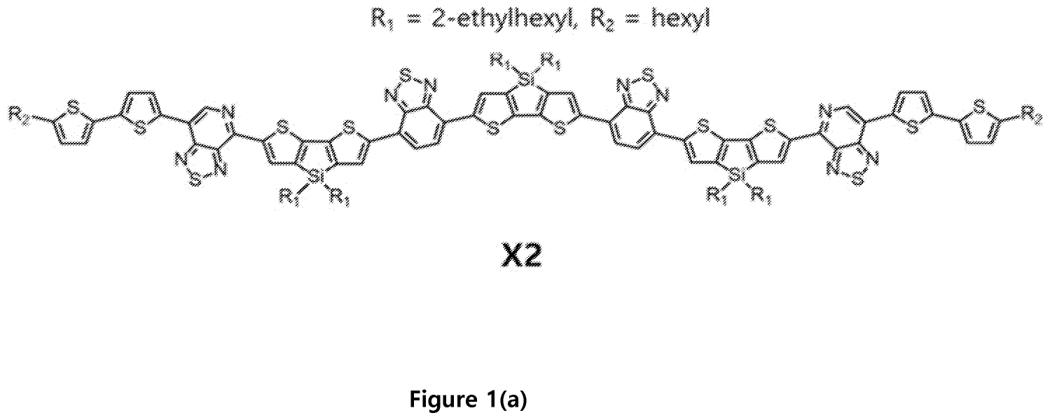

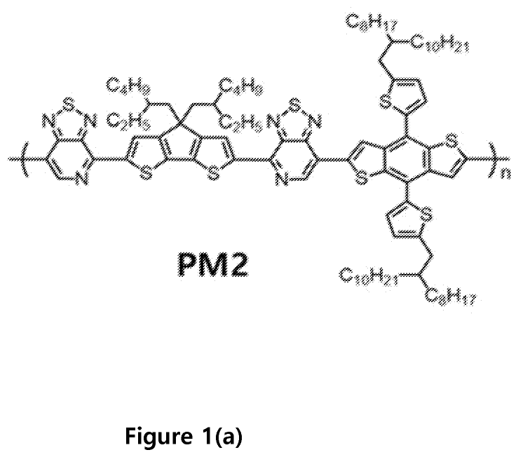

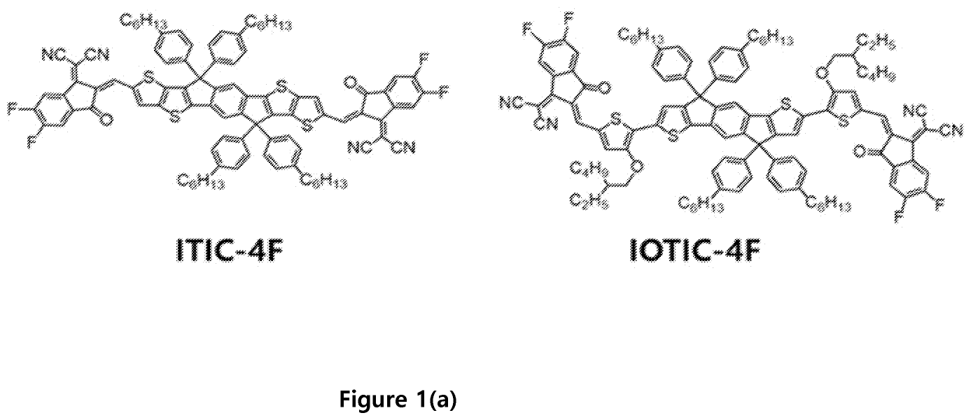

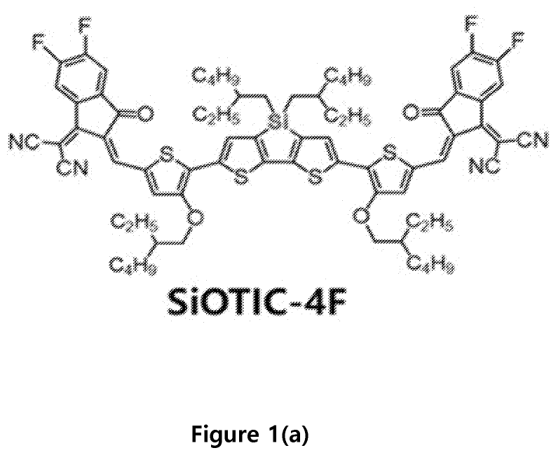

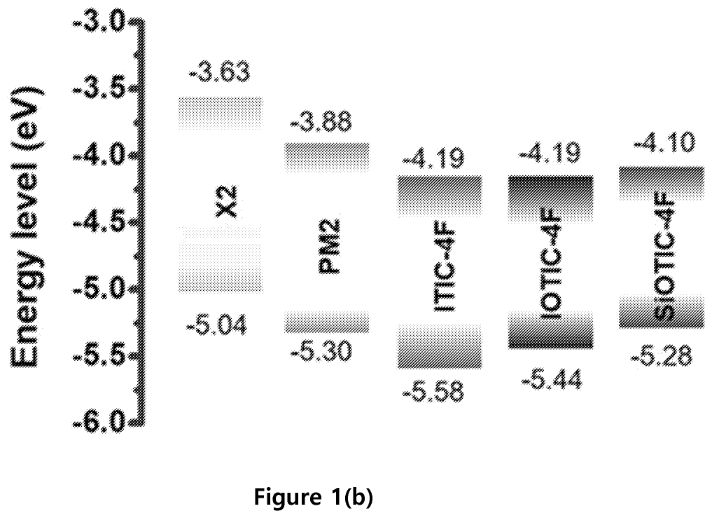

The present disclosure describes electrical property and absorption spectra of multiple NIR non-fullerene acceptors (NFAs) and NIR donors. The molecular structures of donor and acceptors, named X2, PM2, ITIC-4F, IOTIC-4F and SiOTIC-4F, are illustrated in FIG. 1(a) and their energy band diagrams are depicted in FIG. 1(b). Both donors (X2 and PM2) reported in work^{[14-22,27]} have narrow optical energy band gap (E_{g}^{opt}) of around 1.4 eV.

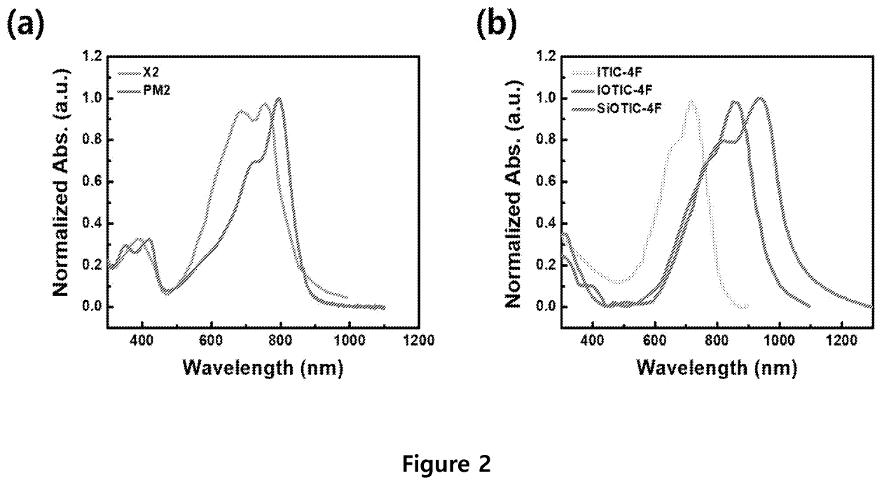

The HOMO energy level and LUMO energy level of X2 are −5.04 eV and −3.63 eV, respectively. While PM2 has −5.30 eV for HOMO energy level and −3.88 eV for LUMO energy level. As shown in FIG. 2(a), two donors displayed absorption spectra up to a NIR wavelength of at least 900 nm, wherein the X2 small molecule has double shoulder peaks at 685 nm and 756 nm due to its strong aggregation in a highly crystalline structure. The PM2 polymer has a maximum absorption peak at 793 nm.

ITIC-4F exhibits an optical bandgap of 1.51 eV and −5.58 eV HOMO and −4.19 eV LUMO. First ITIC-4F, IOTIC-4F and SiOTIC-4F were tested as the non-fullerene acceptors. The ITIC-4F Film exhibits broad absorption with a λₘₐₓ of 717 nm and an E_{g}^{opt} of 1.51 eV. The IOTIC-4F film shows a maximum absorption peak at 856 nm with an absorption onset of ˜995 nm, corresponding to an E_{g}^{opt} of 1.25 eV. Lastly, structured SiOTIC-4F displays an absorption up to at least 1050 nm (E_{g}^{opt} of 1.18 eV) with a maxima peak at 937 nm, which is more redshifted relative to the other two acceptors. The main absorption region of these non-fullerene acceptors was in the wavelength range 700-1000 nm, which is desirable for semitransparent organic photovoltaic (OPV) and photodetector applications. The absorption in the visible light region is much weaker, up to ten times lower (X2 and PM2) than their maximum absorption, or close to zero (IOTIC-4F and SiOTIC-4F), which indicate high device transparency (low optical absorption) in visible light region.

TABLE 1
Optical and electrochemical properties of X2,
PM2, ITIC-4F, IOTIC-4F and SiOTIC-4F.

| Compound | E_{g}^{opt} (eV)^{a} | E_{HOMO} (eV)^{b} | E_{LUMO} (eV)^{c} |
|---|---|---|---|
| X2 | 1.41 | −5.04 | −3.63 |
| PM2 | 1.42 | −5.30 | −3.88 |
| ITIC-4F | 1.51 | −5.58 | −4.19 |
| IOTIC-4F | 1.25 | −5.44 | −4.19 |
| SiOTIC-4F | 1.18 | −5.28 | −4.11^{d} |

^{a}Optical band gap calculated from the absorption edge of thin film.
^{b}HOMO enemy level estimated from the onset oxidation potential.
^{c}LUMO energy level estimated from the onset reduction potential.
^{d}LUMO energy level estimated from following equation E_{LUMO} = E_{g}^{opt} + E_{HOMO}.
Except for SiOTIC-4F, all LUMOS are measured by CV measurement.

A2. Photovoltaic Performance of Bulk-Heterojunction Solar Cells Comprising Active Regions Including Compositions of Matter According to the First Example

2.1. Device Fabrication and Characterization Method

Organic Photovoltaic (OPV) cells are one example of devices that can be used to clarify the sensitiveness of materials' response to photons. A composition comprising of NIR absorbing donor and non-fullerene-based acceptor (NFA) were prepared and tested in an OPV device having a high performance in the near infrared (NIR) region.

The OSC devices based on blends of X2:ITIC-4F and PM2:SiOTIC-4F were fabricated in an inverted device structure of indium-tin-oxide (ITO)/zinc oxide (ZnO)/NIR D:A/MoO₃/Ag. For the OSCs of PM2:IOTIC-4F, the devices had configuration of indium-tin-oxide (ITO)/poly(3,4-ethylenedioxythiophene) polystyrene sulfonate (PEDOT:PSS)/PM2:IOTIC-4F/ZnO nanoparticles (NPs)/Al. The procedures of device fabrication were as follows. Firstly, the ITO-coated glass substrates were cleaned with detergent, then ultra-sonicated in acetone and isopropyl alcohol, and subsequently dried in an oven at 100° C. Then, the cleaned ITO substrates were ultraviolet-ozone treated for 20 min to remove tiny organic residues. The zinc oxide (ZnO) solution was prepared using mixture of diethyl zinc solution in toluene and tetrahydrofuran (THF) (1:2, v/v %) and the ZnO film (ca. 35 nm thick) was spin-coated at 4000 rpm for 15 s and annealed at 110° C. for 15 min. PEDOT:PSS (Baytron P Clevios™ AI 4083, Germany) was spin-coated (ca. 45 nm thick), dried at 140° C. for 10 min. The X2:ITIC-4F blend solution was prepared by dissolving total 17 mg/ml of X2 (1.0 wt %) and ITIC-4F (1.5 wt %) in Chloroform (CF) with and without 0.5 vol % 1,8-diiodooctane or 0.5 vol % 1-chloronaphthalene. The blend solution of PM2:IOTIC-4F as well as the blend solution of PM2:SiOTIC-4F were prepared by dissolving total 20 mg/ml of PM2 (1.0 wt %):IOTIC-4F (1.0 wt %) in chlorobenzene (CB) or total 17 mg/ml of PM2 (1.0 wt %):SiOTIC-4F (1.5 wt %) in CF. These solutions were spin-coated at 2000 to 4000 rpm for film thickness optimization in a nitrogen-filled glove box. In case of conventional device of PM2:IOTIC-4F blend, the ZnO NPs in methanol were deposited on the active layer at 4000 rpm for 20 s. The device was pumped down in vacuum (<10⁻⁶ torr), and MoO₃/Ag (6 nm/100 nm thick) electrode for inverted architecture or A1 (100 nm) electrode for conventional architecture were deposited by thermal evaporation. The active area via using aperture was 9.4 mm². Photovoltaic characteristic measurement was carried out in the glove box using a solar simulator equipped with a Keithley 2635A source measurement unit. J-V curves were measured under AM 1.5G illumination at 100 mW cm². EQE measurement was conducted in nitrogen-filled glove box using an EQE system. The monochromatic light intensity was calibrated using a Si photodiode and chopped at 100 Hz.

A2.2. Photovoltaic Characteristics

The blend of X2 small molecule donor and ITIC-4F non-fullerene acceptor has large HOMO/HOMO offset (˜0.54 eV), an efficient energetic driving force for exciton dissociation. The blends of PM2:SiOTIC-4F and PM2:IOTIC-4F showed much smaller HOMO/HOMO offset of −0.02 eV and 0.14 eV, respectively. Although PM2:SiOTIC-4F has negative value of HOMO_{offset}, it still exhibited decent photovoltaic performance. An empirical 0.3 eV minimum of HOMO/HOMO offset and LUMO/LUMO offset was observed in fullerene-based OSCs for efficient charge carrier generation, separation and extraction. It becomes problematic to maintain such energetic offset when the donor bandgap becomes narrower and narrower into the NIR region. As a consequence, poor carrier generation, extraction, and severe charge recombination is generally observed for NIR donor/fullerene based devices, especially when the bandgap approaches or is narrower than 1.4 eV, which leads to poor device EQE. It is encouraging that driving energies required for efficient charge separation in NIR donor/NFA solar cells described here are smaller than the empirical 0.3 eV observed in fullerene-based OSCs, which is advantageous to minimize energy losses from the difference between optical bandgap, this enables high photocurrents and high voltage to be achieved simultaneously.

These three compositions have sufficient absorption located in the near infrared region up to 1000 nm of wavelength, while simultaneously maintaining high transparency in visible region (400-600 nm), which is suitable for transparent organic photovoltaics. To optimize morphology of the active layers, device fabrication conditions were adjusted by introducing processing additive and changing spin-coating speed for thickness control.

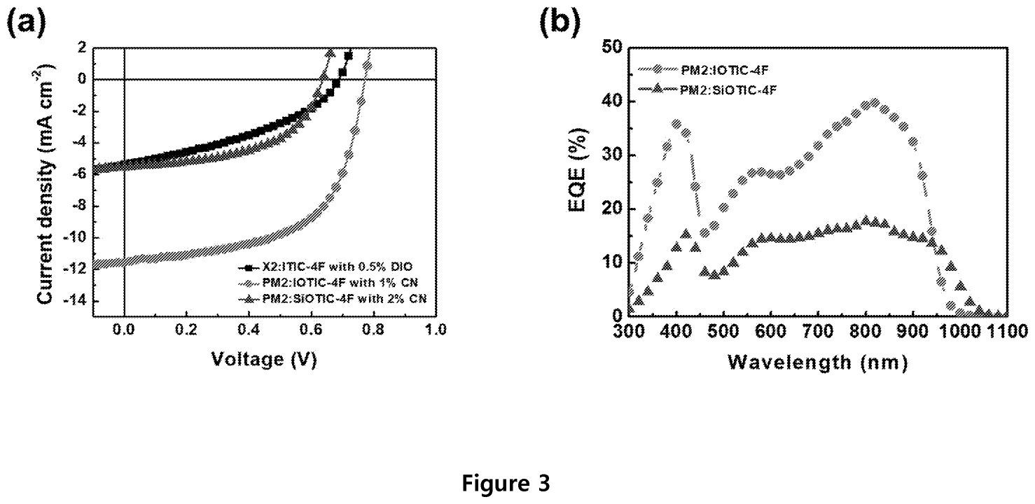

FIG. 3(a) shows current-voltage (J-V) characteristics of the NIR OPVs. Table 2 summarizes the photovoltaic parameters of the optimized OPVs based on X2:ITIC-4F, 10 PM2:IOTIC-4F and PM2:SiOTIC-4F with and without processing additives.

In Current-voltage (J-V) characteristics, the X2:ITIC-4F-based devices exhibited a power conversion efficiency (PCE) of ˜1.43% with J_{SC} of 5.36 mA cm⁻², V_{OC} of 0.69 V and fill factor (FF) of 0.39, which was fabricated with 0.5 vol % DIO additive. Note that X2 and ITIC-4F displayed overlapping absorption in the range of 600-800 nm wavelength, which could be a major factor of the limited J_{SC}. PM2:IOTIC-4F showed higher PCE up to 5.25% arising from enhanced J_{SC} of 11.59 mA cm⁻² by 1 vol % CN additive in solution, V_{OC} of 0.77 V and FF of 0.59. PM2:SiOTIC-4F based OPVs with a HOMO_{offset} close to 0 showed 3.17% PCE with 8.71 mA cm⁻² (J_{SC}), 0.66 V (V_{OC}) and 0.55 (FF).

FIG. 3(b) shows the external quantum efficiency (EQE) spectra of optimal OPV devices with PM2 as donor. The PM2:SiOTIC-4F-based device exhibited broad solar spectral response from 300 to 1050 nm, and PM2:IOTIC-4F-based device showed EQE spectra up to 1000 nm with an EQE maximum of 40% at 820 nm, which agrees with absorption of PM2:NFAs blends. The EQE values have an obvious decrease in the range of 400-600 nm, which indicates the high transparency (low optical absorption) in visible region for the naked eye. Notably, the PM2:IOTIC-4F-based device had the maximum EQE over 700 am and it also showed 24% EQE difference between the minimum EQE and the maximum EQE values.

TABLE 2
Performance of the NIR OPVs

| | | J_{SC} (mA | V_{OC} | FF | PCE |
|---|---|---|---|---|---|
| Blends^{a} | Solvent | cm⁻²) | (V) | (%) | (%)^{c} |
| X2:ITIC- | CF | 4.81 | 0.70 | 0.35 | 1.18 |
| 4F | 99.5% | 5.36 | 0.69 | 0.39 | 1.43 |
| | CF: 0.5% | | | | |
| | DIO | | | | |
| | 99.5% | 4.50 | 0.68 | 0.40 | 1.23 |
| | CF: 0.5% | | | | |
| | CN | | | | |
| PM2:IOTI | CB | 9.39 | 0.81 | 0.53 | 3.99 |
| C-4F | 99% | 11.59 | 0.77 | 0.59 | 5.25 |
| | CB: 1% | | | | |
| | CN | | | | |
| | 99% | 10.66 | 0.76 | 0.59 | 4.77 |
| | CB: 2% CN | | | | |
| PM2:SiOT | CF | 3.29 | 0.69 | 0.45 | 1.03 |
| IC-4F | 98% | 8.71 | 0.66 | 0.55 | 3.17 |
| | CF: 2% CN | | | | |

^{a}X2:ITIC-4F = 1:1.5 (w/w), PM2:IOTIC-4F = 1:1 (w/w) and PM2:SiOTIC-4F = 1:1.5 (w/w)
^{b}Diiodooctane (DIO), chloronaphthalene (CN) were used as processing solvent,
^{c}Highest PCEs.

A.2.3 Electronic Proprieties

The indacenodithiophene (IDT)-based acceptors like ITIC-4F show lower electron mobility (10⁻⁵-10⁻⁴ cm² V⁻¹ s⁻¹) as compared with the fullerene acceptors ((10⁻⁴-10⁻³ cm² V⁻¹ s⁻¹), which may result in an unbalanced electron and hole mobility when blending with high hole mobility donor materials.

A3.1 Effects of Processing Environment

Small molecules may be sensitive to processing environment. Certain treatments such as thermal annealing, solvent annealing and adding processing additives were needed to form moderate crystallinities and adjust domain size. Too high crystallinity and large domain size reduces the charge dissociation efficiency and photovoltaic performance in the bulk heterojunction organic solar cells (OSCs).

The performance of OPVs based on X2:ITIC-4F with various treatments is shown below (see Table 3) Solvent vapor annealing with tetrahydrofuran (THF) gave higher PCE 2%, arising from improved J_{SC} of 7.29 mA/cm², V_{OC} of 0.63, and FF of 0.45.

TABLE 3
Photovoltaic parameters of OPVs fabricated with
various treatments

| | | Spin-rate | J_{SC} | V_{OC} | | PCE |
|---|---|---|---|---|---|---|
| Ratio | Additives | (rpm) | (mA/cm2) | (V) | FF | (%) |
| 4:6 | — | 2000 | 4.81 | 0.70 | 0.35 | 1.18 |
| 4:6 | 0.5% DIO | 2000 | 5.36 | 0.69 | 0.39 | 1.43 |
| 3:7 | 0.5% DIO | 3000 | 5.22 | 0.67 | 0.38 | 1.35 |
| 3:7 | 1% DIO | 3000 | 5.41 | 0.67 | 0.41 | 1.49 |
| | Solvent | | | | | |
| | vapor | Time | J_{SC} | V_{OC} | | PCE |
| Ratio | annealing | (min) | (mA/cm2) | (V) | FF | (%) |
| 3:7 | CF | 1 | 4.38 | 0.65 | 0.31 | 0.97 |
| | | 3 | 6.92 | 0.70 | 0.38 | 1.85 |
| | THF | 1 | 4.22 | 0.69 | 0.37 | 1.09 |
| | | 3 | 7.29 | 0.63 | 0.45 | 2.03 |
| | DCM | 1 | 4.63 | 0.59 | 0.33 | 0.91 |
| | | 3 | 4.96 | 0.66 | 0.35 | 1.14 |

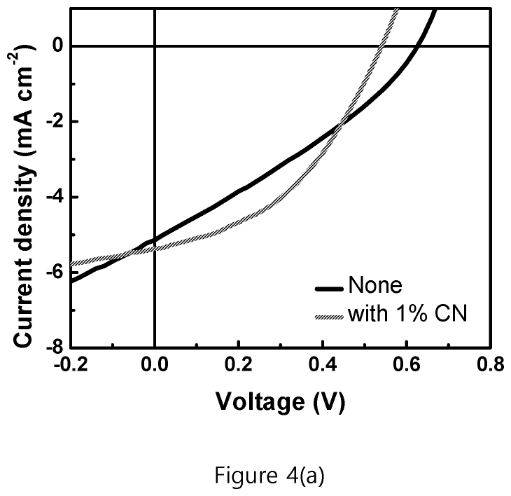

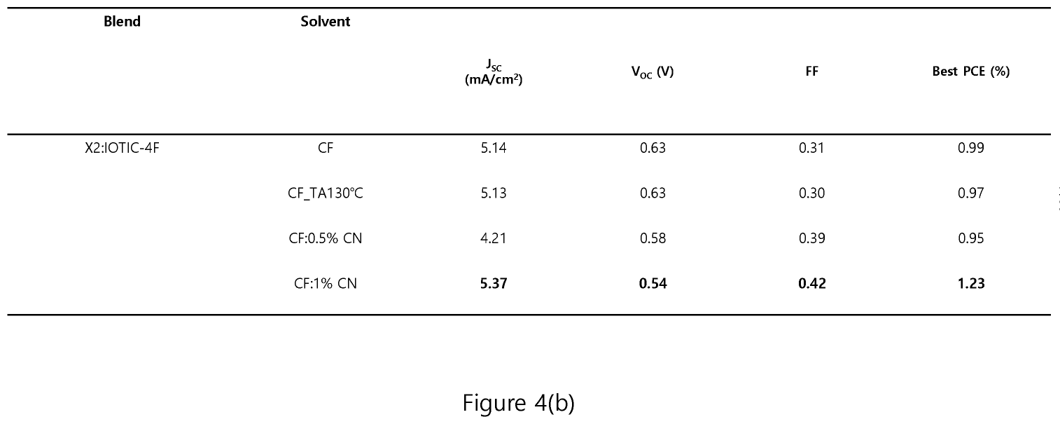

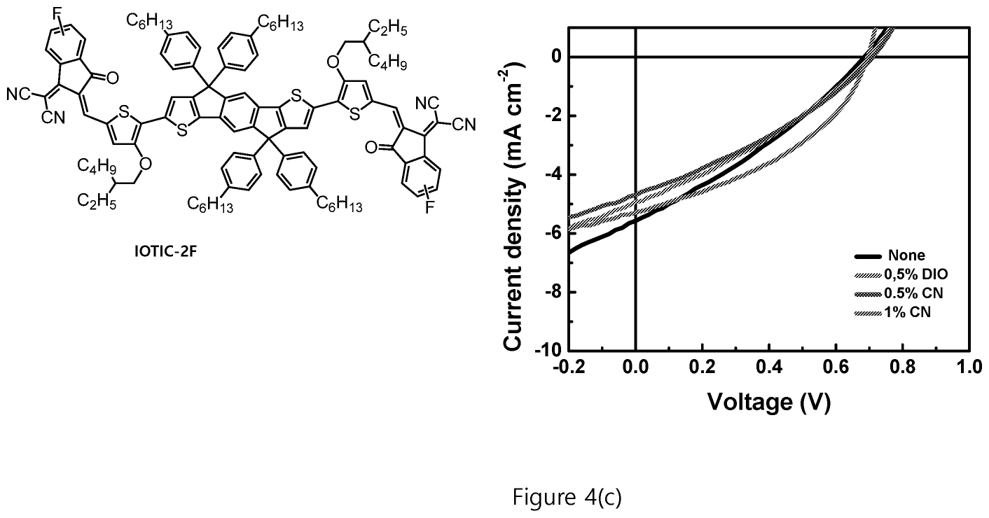

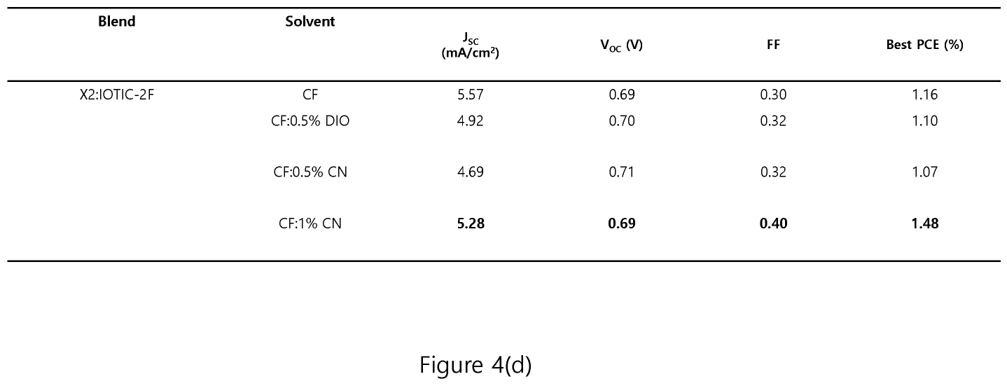

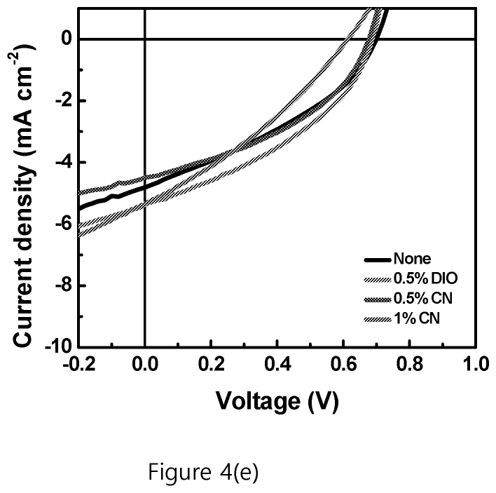

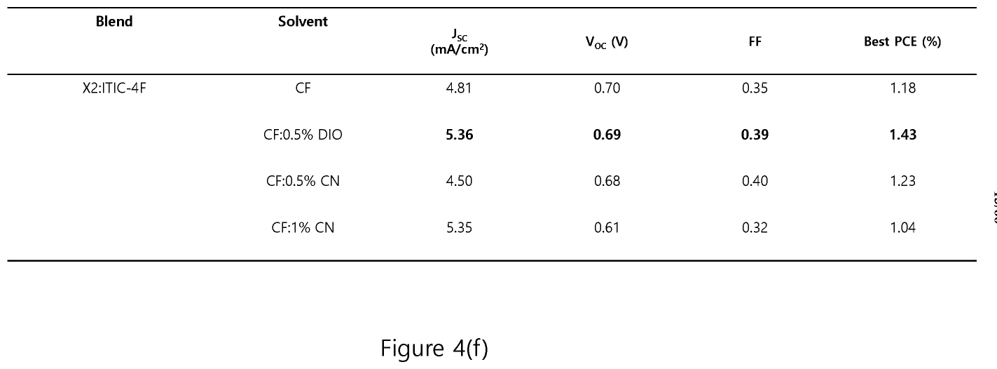

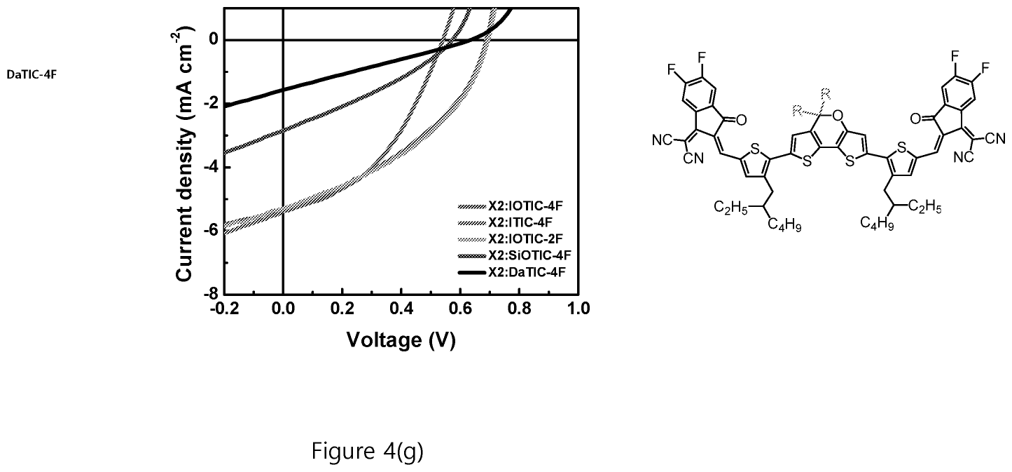

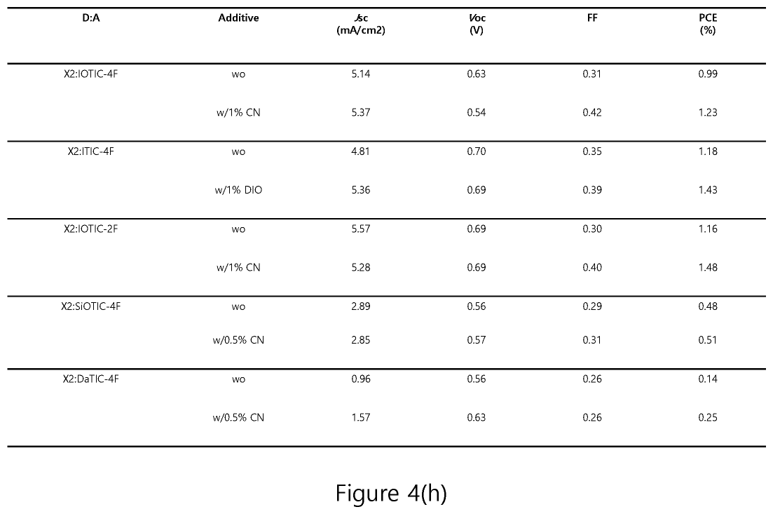

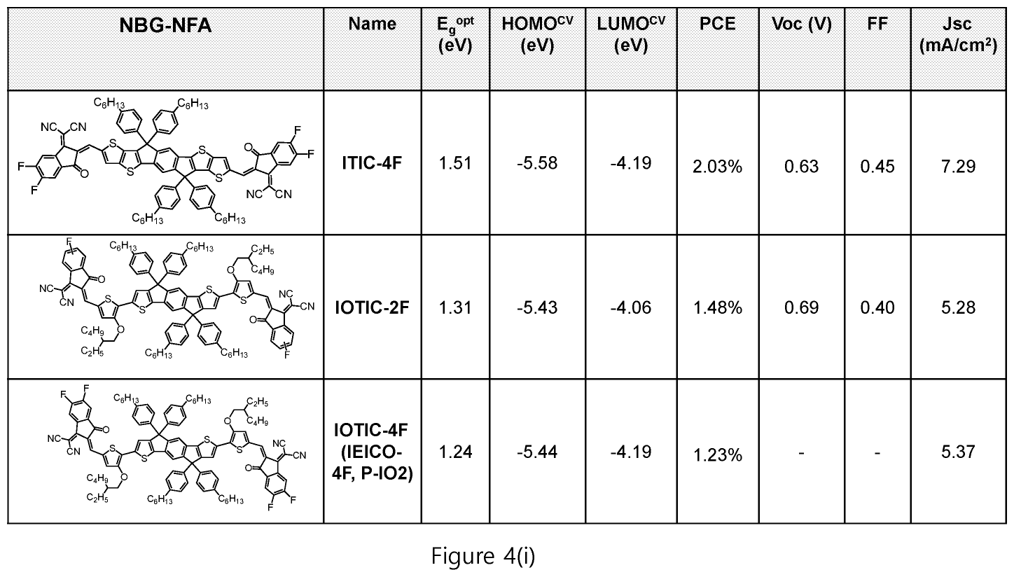

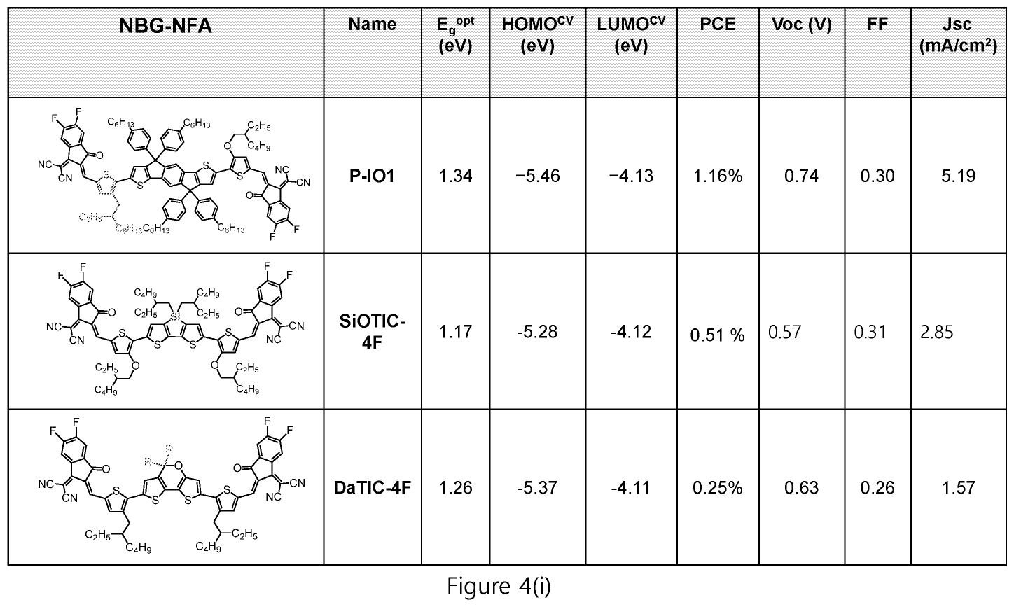

FIGS. 4(a)-(b) illustrate device performance as a function of processing conditions for a device structure comprising an active region comprising X2:IOTIC-4F. The device was fabricated as follows with the following device structure: ITO/PEDOT:PSS/Active/ZnO NPs/Al; PEDOT:PSS: AI4083, 4000 rpm, 40s_TA 140° C. 10 min; ZnO NPs: Dissolved in MeOH 4000 pm, 30s_TA 110° C. 10 min; Active layer: D:A (1:1.5) 17 mg/ml in CF without/with CN (0.5% and 1% v/v) additive; and Al: 100 nm by thermal evaporation.

FIGS. 4(c)-(d) illustrate device performance as a function of processing conditions for a device structure comprising an active region comprising X2:IOTIC-2F. The device was fabricated as follows with the following device structure: ITO/ZnO/Active/MoOx/Ag. ZnO: Diethylzinc solution: THF (2:1 v/v %) 4000 rpm, 20s_TA 110° C. 10 min. Active layer: D:A (1:1.5) 17 mg/ml in CF without/with CN and DIO (0.5%, 1% v/v) additive. MoOx/Ag: 7 nm, 90 nm by thermal evaporation.

FIG. 4(e)-(f) illustrates device performance as a function of processing conditions for a device structure comprising an active region comprising X2:ITIC-4F. The device was fabricated as follows with the following device structure: ITO/ZnO/Active/MoOx/Ag ZnO: Diethylzinc solution: THF (2:1 v/v %) 4000 rpm, 20s_TA 110° C. 10 min. Active layer: D:A (1:1.5) 17 mg/ml in CF without/with CN and DIO (0.5%, 1% v/v) additive. MoOx/Ag: 7 nm, 90 nm by thermal evaporation.

FIG. 4(h-i) summarize performance with donor X2 as a function of NFA.

A.3.2 Bi Layer Structure Photovoltaic Performance

To confirm which materials are dominantly affected by additional treatment, bi-layer structured OPVs were fabricated and tested. We tested the effect of adding DIO processing additive, and THF-based solvent vapor annealing (SVA) for both donor layer and electron layer, respectively. Taking into account of material solubility and device configuration, the OPVs were fabricated with conventional structure (ITO/MoO₃/Donor/Acceptor/Al). the OPV performance is listed in Table 4. For the best result, X2 required DIO processing additive and solvent vapor annealing, while, ITIC-4F needed only solvent vapor annealing. This indicates that X2 needs molecular re-ordering in the film for carrier balance with electron movement in ITIC-4F domains.

TABLE 4
Bi-layer structured photovoltaic performance.

| Donor | Acceptor | J_{SC} | V_{OC} | | PCE |
|---|---|---|---|---|---|
| condition | condition | (mA/cm2) | (V) | FF | (%) |
| X2 | ITIC-4F | 2.45 | 0.46 | 0.37 | 0.42 |
| | ITIC-4F with | 1.75 | 0.43 | 0.35 | 0.27 |
| | 0.5% DIO | | | | |
| | ITIC-4F with | 2.00 | 0.62 | 0.30 | 0.38 |
| | THE SVA-1 min | | | | |
| X2 with 0.5% | ITIC-4F | 3.92 | 0.45 | 0.41 | 0.73 |
| DIO | ITIC-4F with | 3.36 | 0.42 | 0.36 | 0.51 |
| | 0.5% DIO | | | | |
| | ITIC-4F with | 1.73 | 0.47 | 0.25 | 0.21 |
| | THF SVA-1 min | | | | |
| X2 with THF | ITIC-4F | 3.49 | 0.37 | 0.32 | 0.42 |
| SVA-1 min | ITIC-4F with | 3.28 | 0.48 | 0.36 | 0.56 |
| | 0.5% DIO | | | | |
| | ITIC-4F with | 3.43 | 0.60 | 0.34 | 0.70 |
| | THF SVA-1 min | | | | |
| X2 with THF | ITIC-4F | 3.62 | 0.53 | 0.38 | 0.74 |
| SVA-3min | ITIC-4F with | 3.00 | 0.49 | 0.36 | 0.53 |
| | 0.5% DIO | | | | |
| | ITIC-4F with | 3.56 | 0.59 | 0.36 | 0.77 |
| | THF SVA-1 min | | | | |

A.4 Device Performance with Donor PM2 as Function of NFA

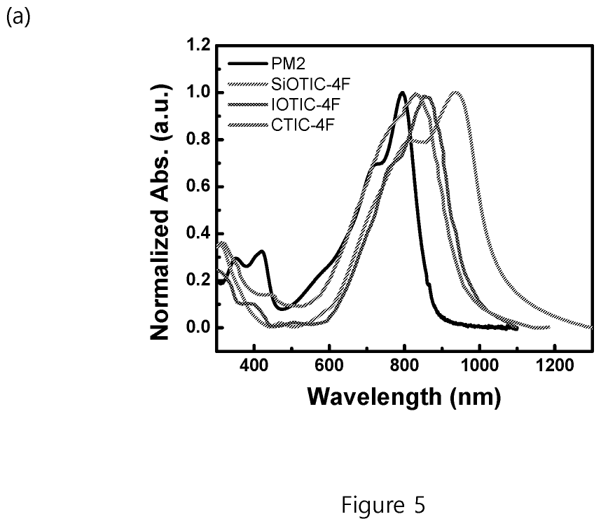

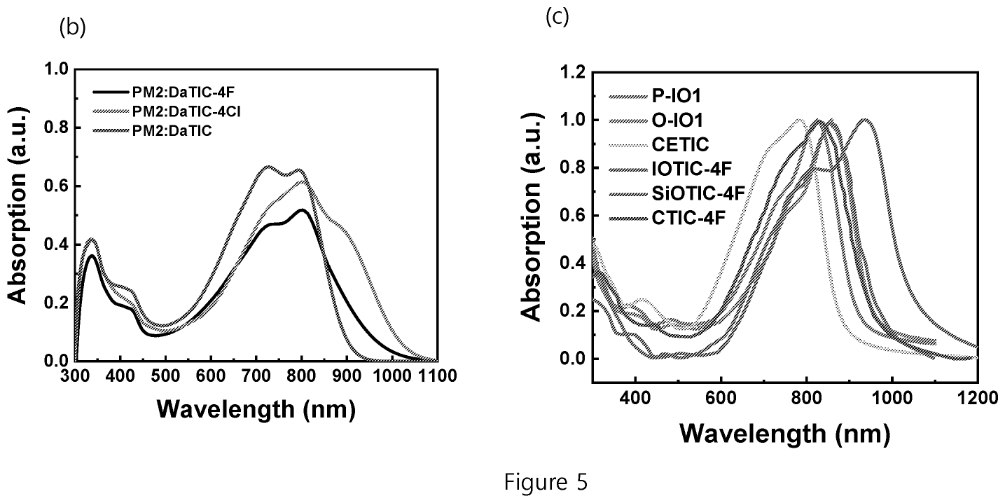

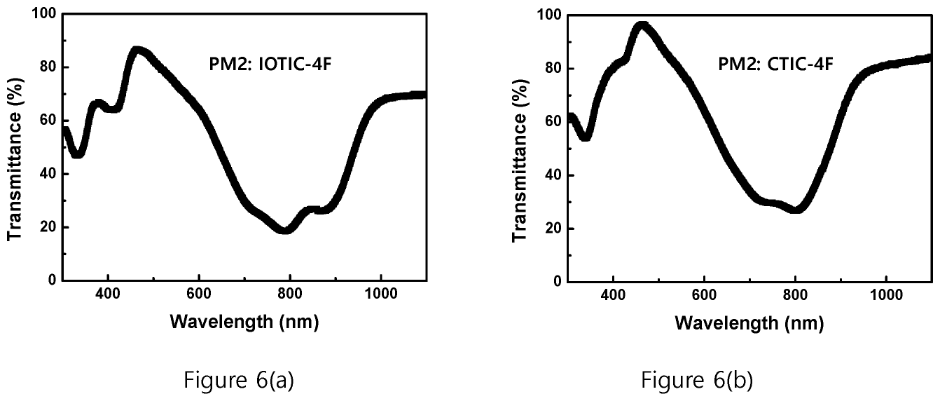

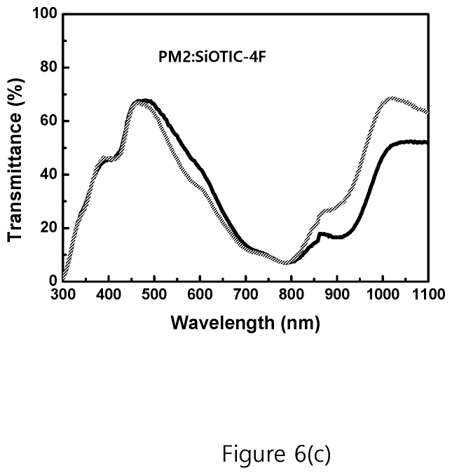

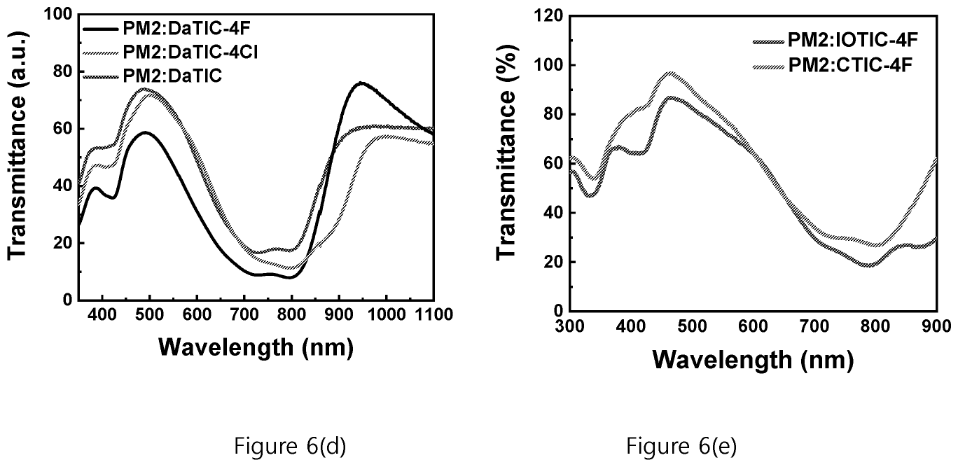

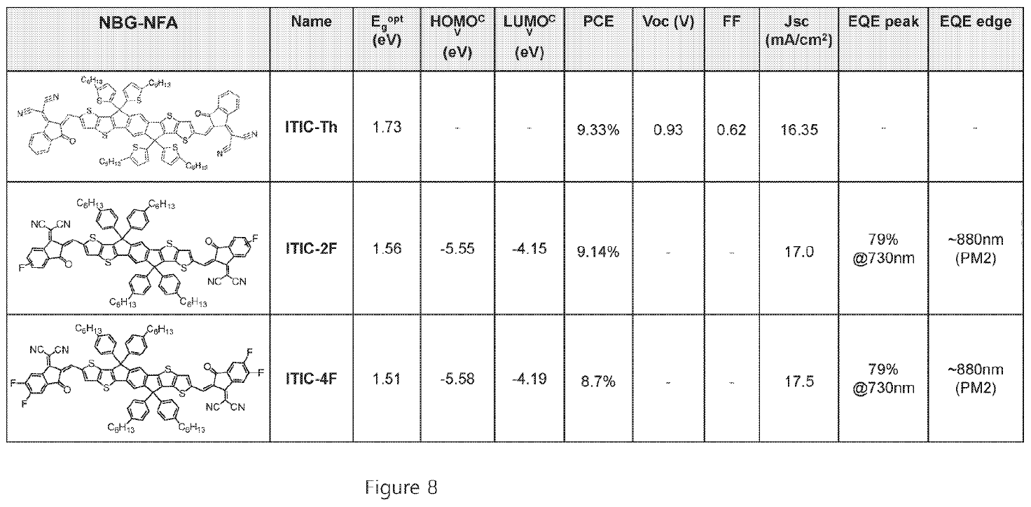

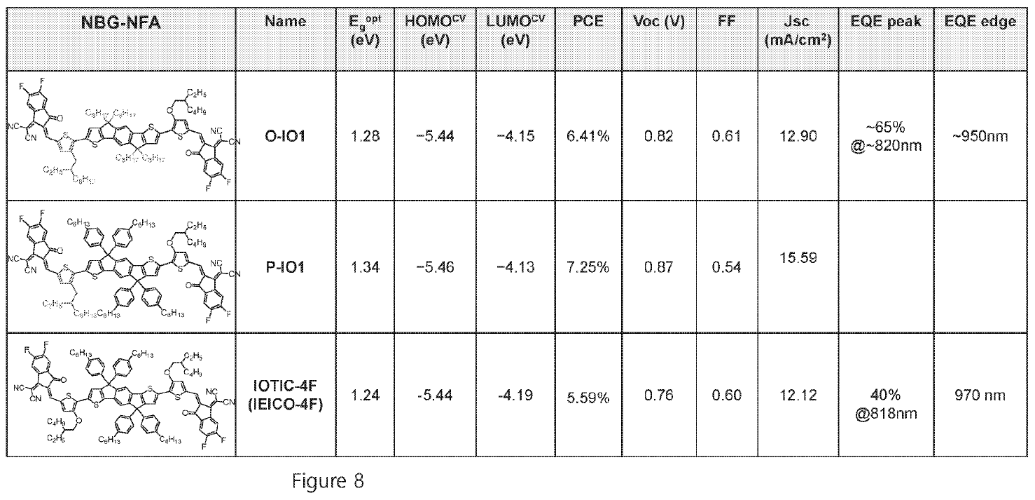

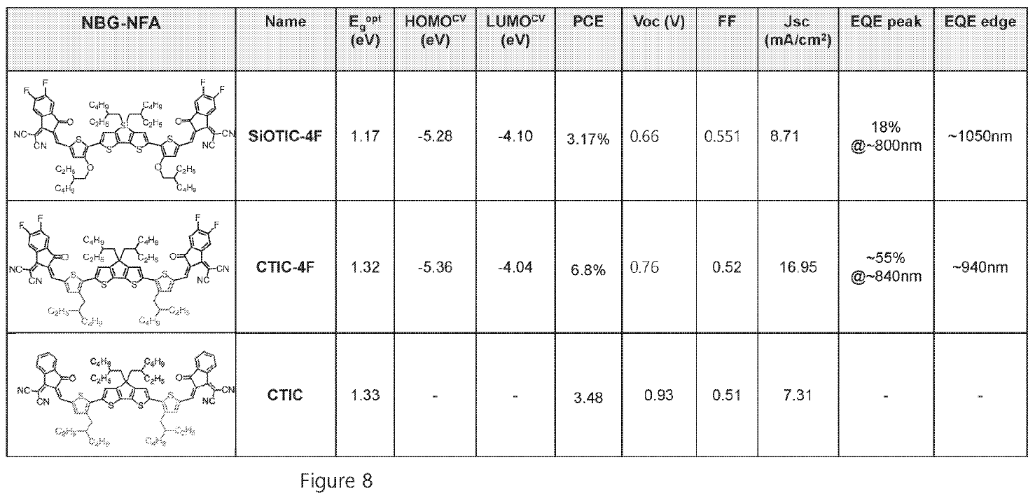

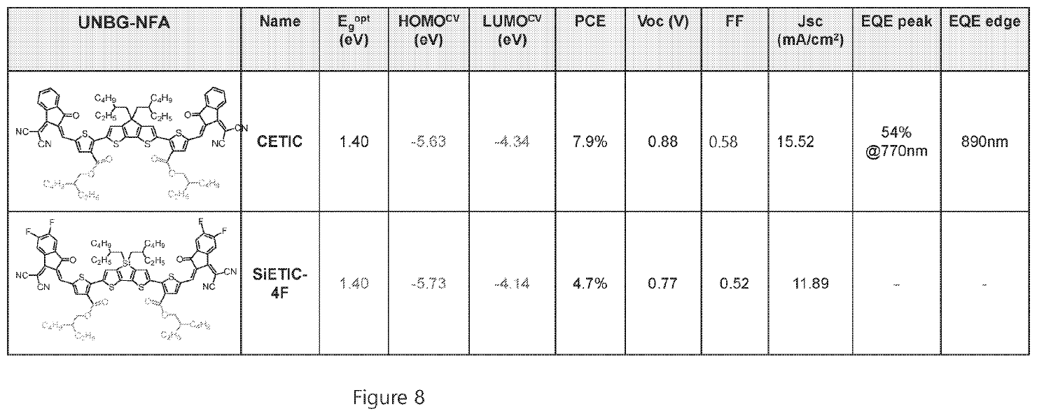

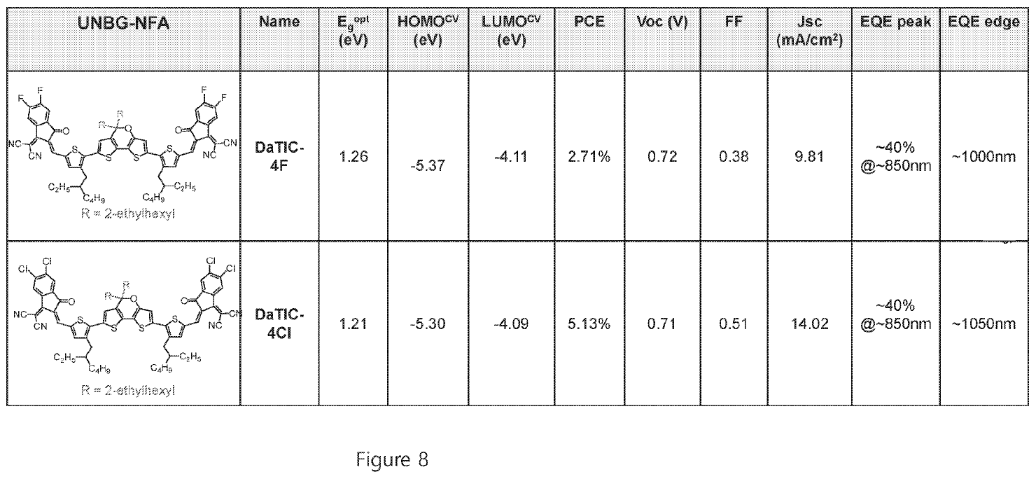

Photovoltaic properties of PM2 with various NFAs with bandgap in the range of 1.1-1.4 eV were tested either in a device or an inverted device structure. The NFA bandgaps, HOMO-LUMO levels, device V_{oc}, J_{sc}, FFs, PCEs, EQE peaks and EQE edges are summarized in tabular form in FIG. 8. Device absorption spectra, transmittance spectra, J-V characteristics and EQE spectra are summarized in FIGS. 5-7, respectively. The summary demonstrates that PM2 is a suitable donor for all type of NIR non-fullerene acceptors, which gives high J_{sc}, V_{oc}, solar cell efficiencies and transparency. EQE maximum up to 80% in the NIR region and EQE responses beyond 1000 nm are demonstrated. Device transmittance over 50% to 70% are achieved in the visible light region (400-600 nm), with maximum transmittance close to 100%.

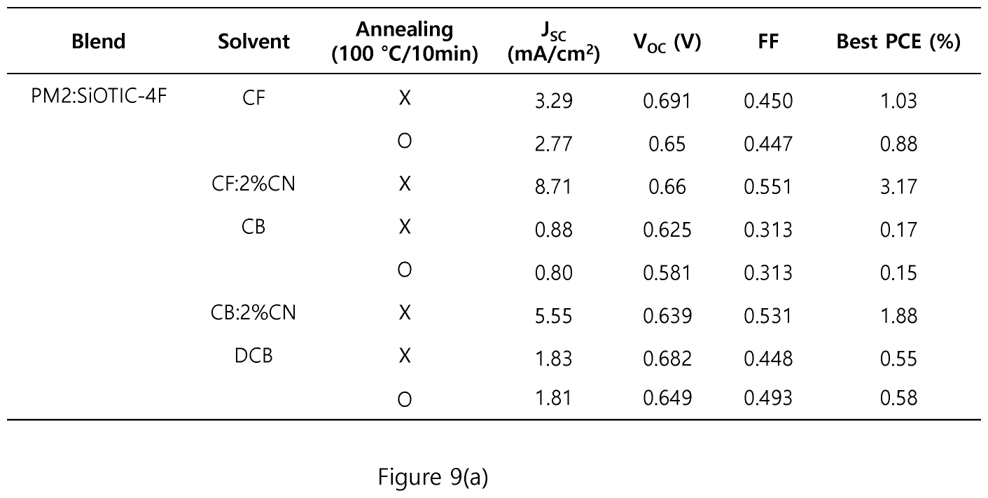

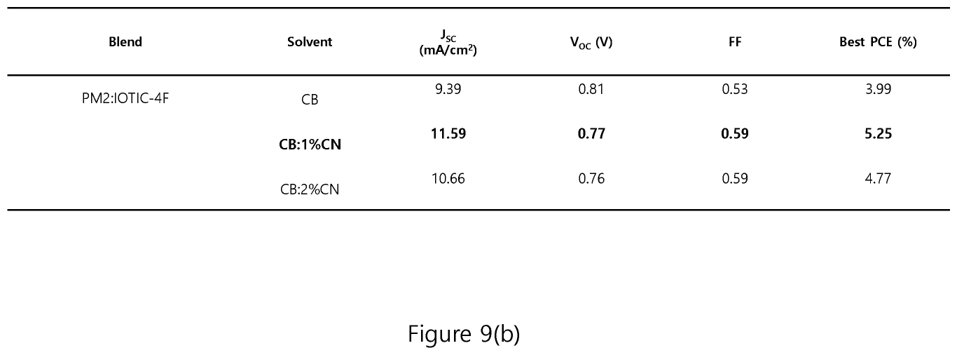

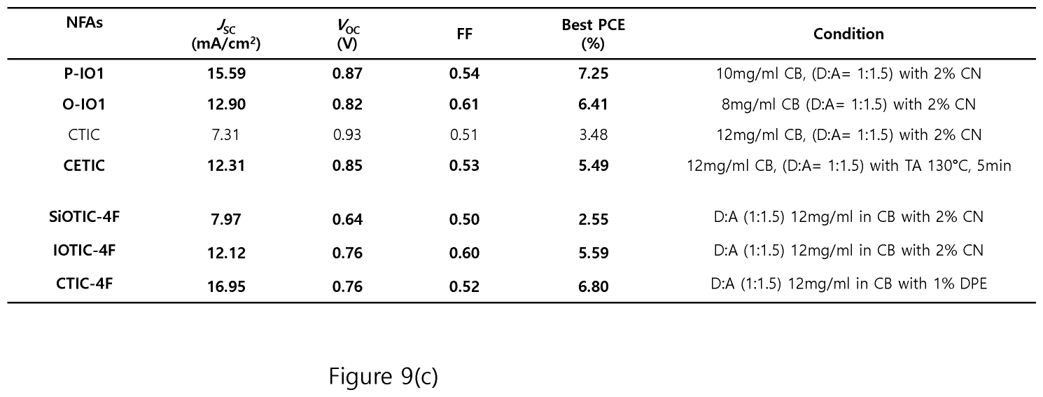

FIG. 9(a) illustrates the device performance for PM2:SiOTIC-4F blend for various processing conditions and for the device structure: ITO/ZnO/Active/MoOx/Ag, ZnO: Diethylzinc solution: THE (2:1 v/v %) 4000 rpm, 20s_TA 110° C. 10 min, Active layer: D:A (1:1.5) 12 mg/ml in CF and CB without/with CN (2% v/v) additive. MoOx/Ag: 7 nm, 90 nm by thermal evaporation.

FIG. 9(b) illustrates the device performance for PM2:IOTIC-4F blend for various processing conditions and for the device structure: ITO/PEDOT:PSS/Active/ZnO NPs/Al PEDOT:PSS: AI4083, 4000 rpm, 40s_TA 140° C. 10 min, ZnO NPs: Dissolved in MeOH 4000 pm, 30s_TA 110° C. 10 min, Active layer: D:A (1:1) 10 mg/ml in CB without/with CN (1% and 2% v/v) additive, Al: 100 nm by thermal evaporation.

FIG. 9(c) illustrates a summary of the OPV device performance using PM2 as a function of device processing conditions.

A.5 Photodetector Performance of Bulk-Heterojunction Devices

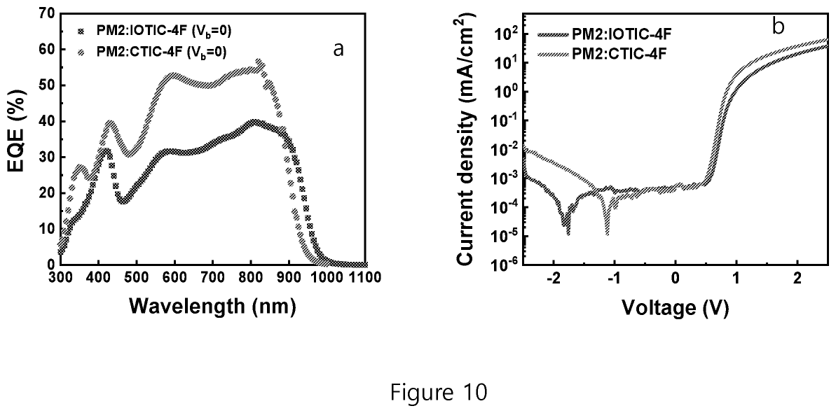

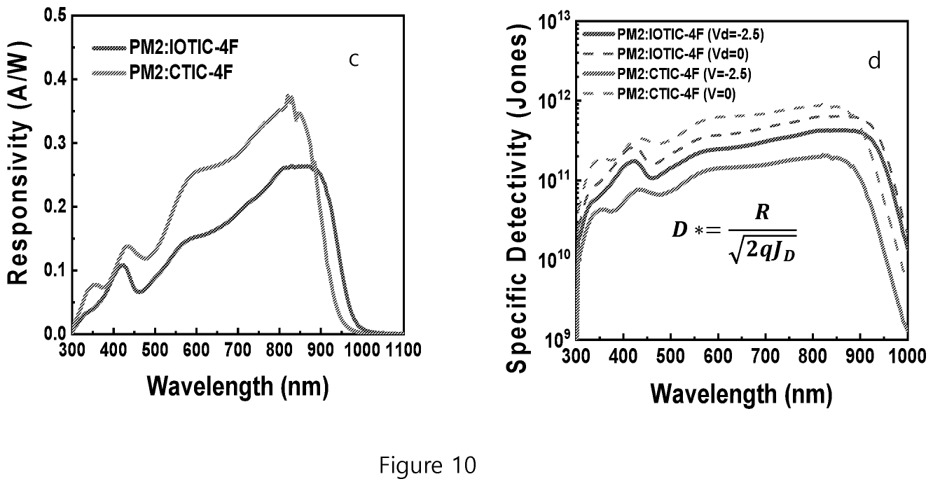

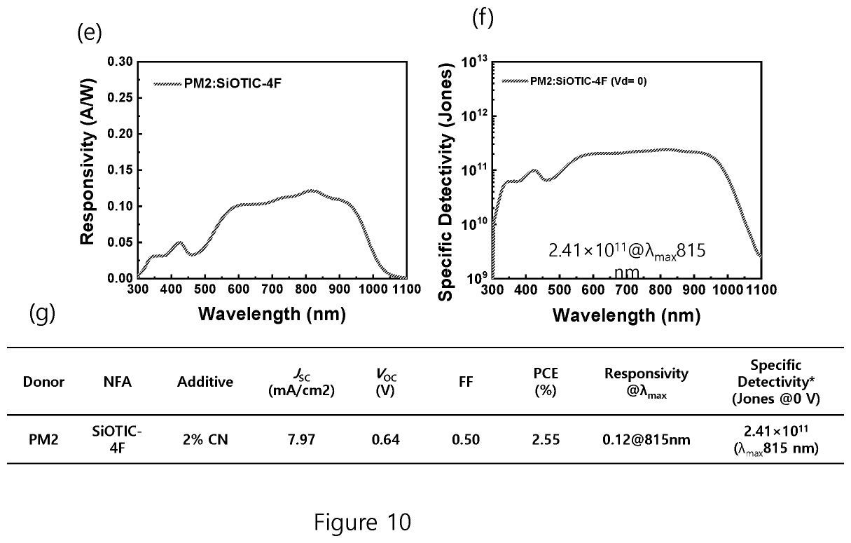

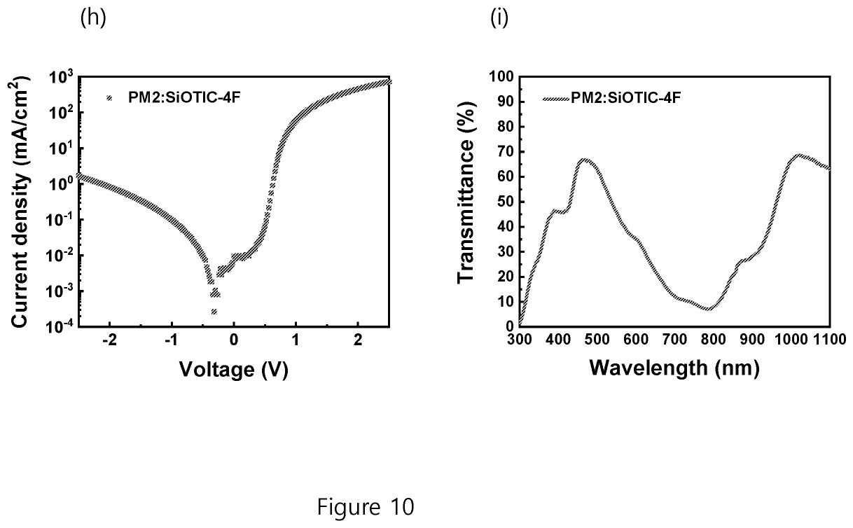

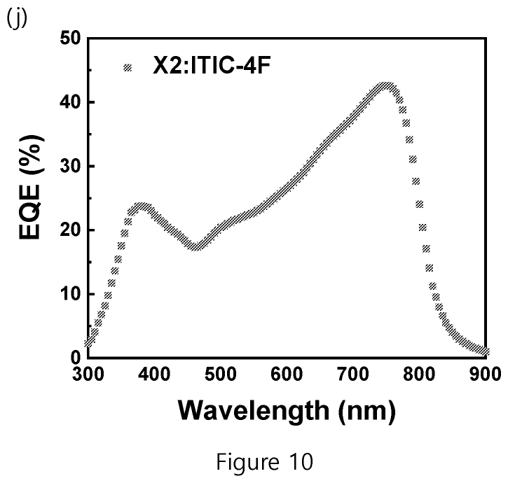

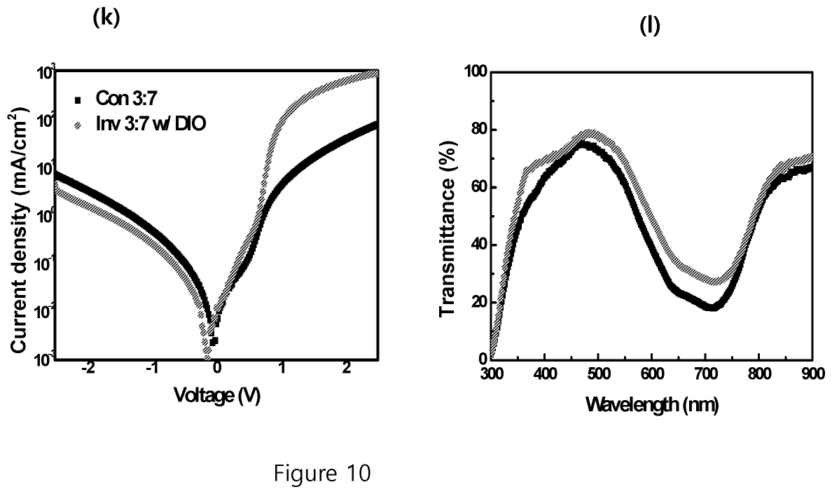

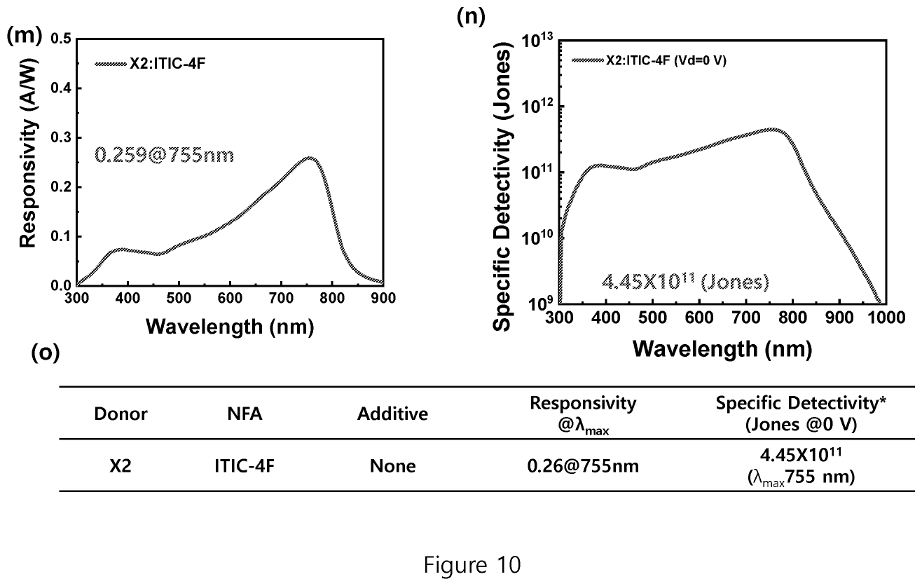

Taking advantage of the optoelectronic properties of PM2:NFA and X2:NFA, we also fabricated efficient NIR organic photodetectors. The responsivity (R), which is an important parameter for evaluating the light-responding performance of a photodetector, is defined as the ratio of photocurrent to the incident light intensity, and can be calculated from the EQE according to the following equation:

$$R = \frac{{J}_{ph}}{{I}_{light}} = \frac{EQE λ}{1240}$$

where Jₚₕ is the photocurrent density in A/cm², I_{light} is the incident light intensity in W/cm², λ is the wavelength. FIG. 10c shows the spectral responsivity of the bulk heterojunction (BHJ) photodiodes based on PM2:IOTIC-4F and PM2:CTIC-4F, respectively. FIG. 10e shows the spectral responsivity of the BHJ photodiodes based on PM2:SiOTIC-4F. FIG. 10(m) shows the spectral responsivity of the BHJ photodiodes based on X2:ITIC-4F. All devices showed broad photo-response across the visible to NIR region. The maximum responsivity of 0.26, 0.36 A/W and 0.12 A/W was found at 860 nm, 830 nm and 815 nm for PM2:IOTIC-4F, PM2:CTIC-4F and PM2:SiOTIC-4F under short-circuit condition, respectively. The maximum responsivity of 0.26 A/W was found at 755 nm for X2:ITIC-4F under short-circuit condition. When the photodetectors are operated under reverse bias, responsivity is expected to further increase as a result of more efficient charge collection under external electric field.

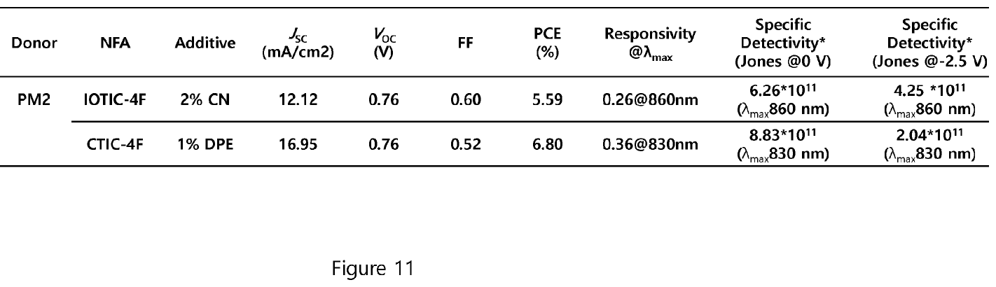

In addition to responsivity, another critical figure of merit for the photodetector is specific detectivity (D*), which evaluates the sensitivity of a photodetector to weak optical signals. The shot noise-limited specific detectivity can be calculated from the responsivity and dark J-V characteristics. The D* of the photodetectors is shown in FIG. 10d, 10f and FIG. 10(n). All devices showed broad photo-response across the visible to NIR region. At 0 V, specific detectivity of 6.26×10¹¹ at 860 nm, 8.83×10¹¹ jones at 830 nm, 2.41×10¹¹ at 815 nm and 4.45×10¹¹ jones at 755 nm are obtained for PM2:IOTIC-4F, PM2:CTIC-4F, PM2:SiOTIC-4F and X2:ITIC-4F based devices. As the reverse bias increases, the D* decreases for NFAs based photodetectors. At −2.5 V, the corresponding values are 4.25×10¹¹ and 2.04×10¹¹ jones for PM2:IOTIC-4F and PM2:CTIC-4F, respectively. This is due to positive effect of increasing R is outweighed by the negative effect of increasing the Ja, and thus the noise, under larger reverse bias. This suggests the limiting factor of detectivity performance is mainly associated with dark current and interface engineering are being carried out to minimize the dark current under reverse bias. The device fabrication conditions and photodetector properties are further summarized in FIG. 11 (Device structure: ITO/ZnO/Active/MoOx/Ag; ZnO: Diethylzinc solution: THF (2:1 v/v %) 4000 rpm, 20s_TA 110° C. 10 min; Active layer: D:A (1:1.5) 12 mg/ml in CB with CN (2% v/v) additive and TA 110° C. for PM2:IOTIC-4F), D:A (1:1.5) 12 mg/ml in CB with DPE (1% v/v) additive for PM2:CTIC-4F; and MoOx/Ag: 7 nm, 90 nm by thermal evaporation.

### B. Second Example: NFAs Combined with P2 Type Polymers

Since the active layer absorption in fullerene based solar cells is mainly in the range from 300 nm to 800 nm, utilization of the near infrared (NIR) region of the solar spectrum has been limited.^{[5-8]} Till now semitransparent organic solar cells have limited usage as an energy source for Internet of Things (IoT) generation and applications such as vehicle windows and smart building exteriors or windows. Moreover, since 50% of the sunlight at the earth's surface comprises infrared (IR) radiation intensity, ideal organic solar cells as next generation energy sources require narrow bandgap materials.

The combination of NIR donor and NIR NFAs described herein may be used realize high performance solar cells with high transparency. As described herein, by tuning their optical and electrical properties, donors and acceptors can absorb in the near infrared (NIR) region of the electromagnetic spectrum. In this regard, P2 was chosen as a narrow band gap material (bandgap less than 1.12 eV; HOMO energy level and LUMO energy level of P2 are −5.16 eV and −3.70 eV) to combine with NIR non-fullerene acceptors exhibiting a narrow bandgap of less than 1.30 eV. Such compositions of P2:COTIC-2F, P2:CO6IC, P2:C2P1, P2:GeOTIC-4F-EH and P2:GeOTIC-4F-BO can be applied to NIR organic solar cells and photodetectors. These narrow band gap devices are shown here to exhibit relatively higher transparency in the visible region as compared to fullerene-based organic solar cells^{[2-4]} while also exhibiting strong absorption of photons in the NIR region.

In a recent study, it was shown that crystallization of molecular materials is often crucial for ensuring intermolecular interactions and phase separation required for the efficient electron extraction. Thus, additional processing methods, i.e. solvent additives, thermal annealing (TA) are required to kinetically favor crystallization and phase separation^{[14-17]}.

B1. Optical and Electrical Property of NIR Materials According to the Second Example

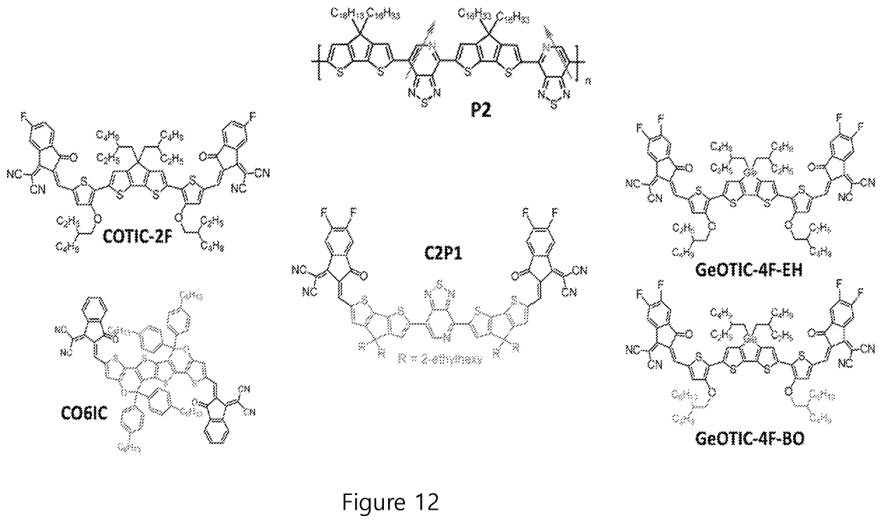

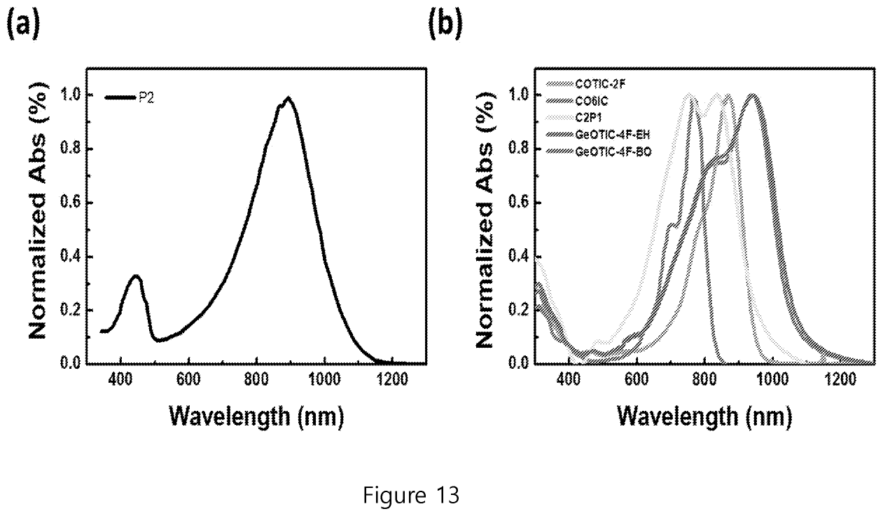

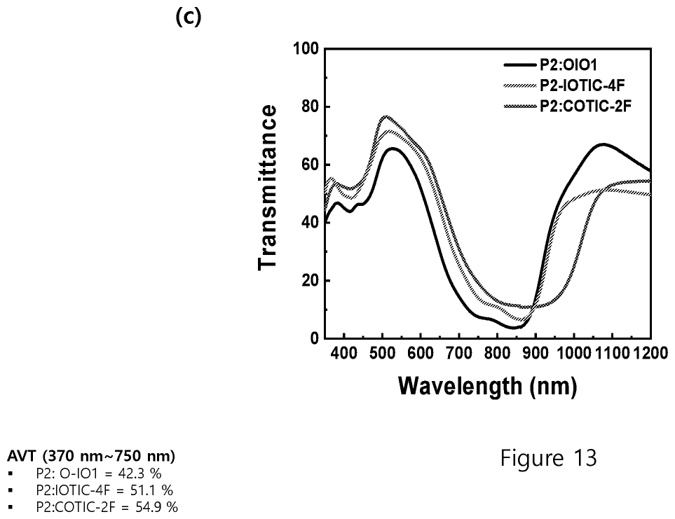

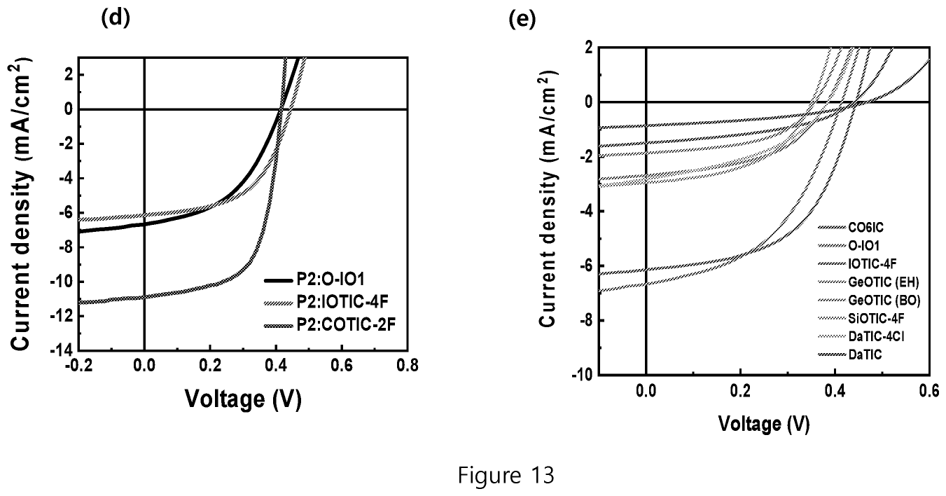

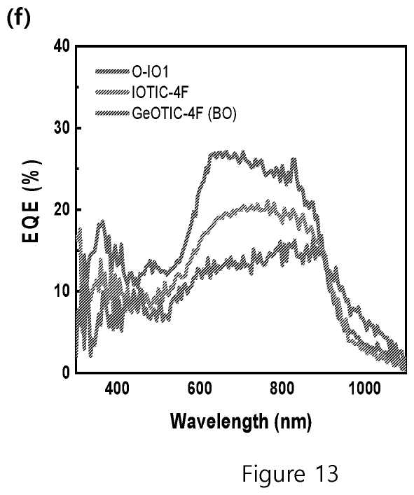

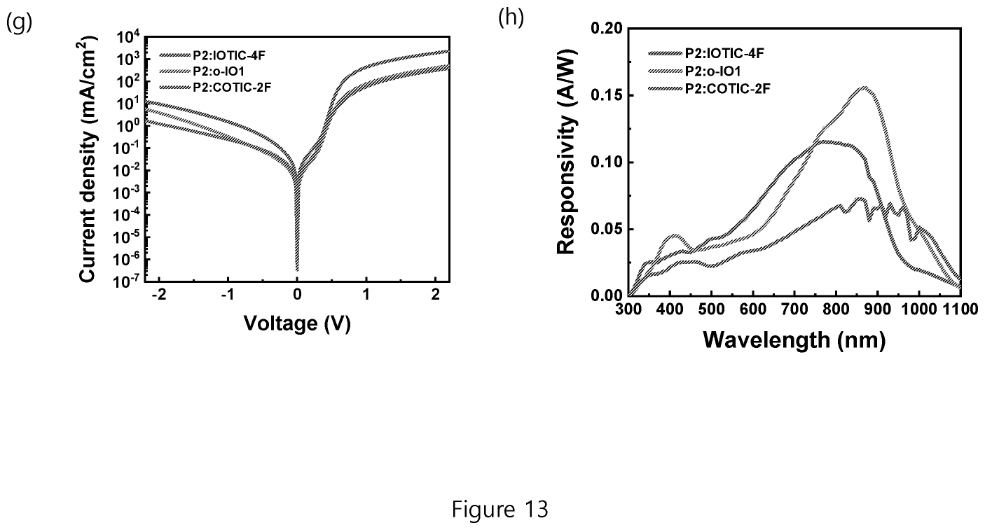

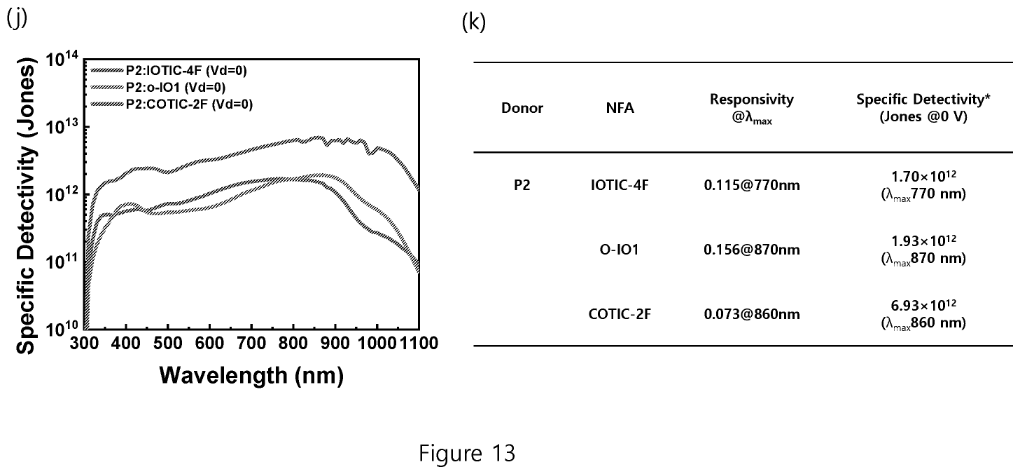

This example describes electrical property and absorption spectra of donor P2 and acceptors (COTIC-2F, CO6IC, C2P1, GeOTIC-4F-EH and GeOTIC-4F-BO). The chemical structures are illustrated in FIG. 12 and their absorptions are depicted in FIG. 13. As shown in FIG. 13, the P2 polymer donor absorbs up to a NIR wavelength of at least 1100 nm of near infrared region and has a maximum absorption peak at 885 nm (strong main absorption peak between 700-1000 nm, low absorption in the 400-600 nm range).

The COTIC-2F acceptor exhibits broadened absorption with a λₘₐₓ of 960 nm and an E_{g}^{opt} of 1.29 eV in solution. The COTIC-2F film shows a maximum absorption peak at 982 nm with an absorption onset of ˜1160 nm (E_{g}^{opt} ˜1.07 eV). The CO6IC acceptor exhibits broadened absorption with a λₘₐₓ of 760 nm and an E_{g}^{opt} of 1.50 eV in solution. The C2P1 solution shows a maximum absorption peak at 755 nm and 833 nm with an absorption onset of ˜968 nm, corresponding to an E_{g}^{opt} of 1.28 eV. Lastly, structured GeOTIC-4F series materials display absorption until 1060 nm in solution (E_{g}^{opt} of 1.14 and 1.17 eV, respectively) with a maxima peak at 940 nm. The main absorption region of these non-fullerene acceptors is at 700-1000 nm with weak or close to zero absorption in the visible light range, which is desirable for semitransparent and transparent organic photovoltaic (OPV) and photodetector applications. FIGS. 13(g)-(i) OPD device performance including responsivity (g) specific detectivity (h) and (i) tabulated data for PM2:SiOTIC-4F photodetectors.

TABLE 5
Optical and electrochemical properties of P2, COTIC-2F,
CO6IC, C2P1, GeOTIC-4F-EH and GeOTIC-4F-BO.

| compd | E_{g}^{opt} (eV)^{a} | E_{HOMO} (eV)^{b} | E_{LUMO} (eV)^{c} |
|---|---|---|---|
| P2 | 1.12 | −5.16 | −3.70 |
| COTIC-2F | 1.29 | −5.20 | −4.11 |
| CO6IC | 1.50 | −5.57 | −4.17 |
| C2P1 | 1.28 | — | — |
| GeOTIC-4F-EH | 1.14 | −5.51 | −4.03 |
| GeOTIC-4F-BO | 1.17 | — | — |

^{a}Optical band gap calculated from the absorption edge of solution (materials are dissolved in chloroform (CF)).
^{b}HOMO energy level estimated from the onset oxidation potential.
^{c}LUMO energy level estimated from the onset reduction potential.

B2. Device Fabrication and Characterization Method

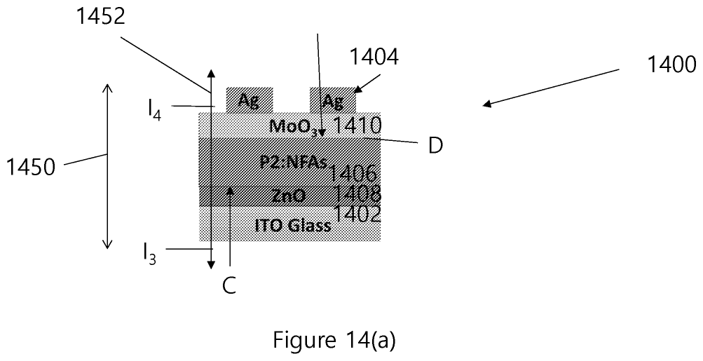

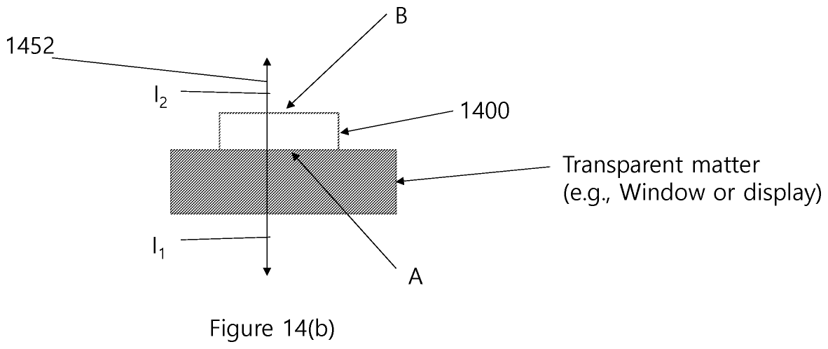

FIG. 14(a) illustrates a photosensitive device 1400 (solar cell or photodetector) comprising a cathode 1402; an anode 1404: both the cathode and anode can be transparent in some embodiments; and the active region or layer 1406 having a thickness between the cathode and the anode. The device further includes a hole blocking layer 1408 and an electron blocking layer 1410. Both the charge blocking layers can be transparent in some embodiments; In one or more examples, the device has a thickness 1450 of 3 micrometers or less. FIG. 14(b) illustrates the device on a transparent matter (e.g., window). The device and or the device and the window can be transparent to electromagnetic radiation 1452 having visible wavelengths (e.g., in a range of 400 nm-600 nm).

The OSC devices based on blends of P2:COTIC-2F, P2:CO6IC, P2:C2P1, P2:GeOTIC-4F-EH and P2:GeOTIC-4F-BO were fabricated in an inverted device structure of indium-tin-oxide (ITO)/zinc oxide (ZnO)/NIR D:A/MoO₃/Ag or conventional device structure of ITO/PEDOT:PSS/NIR D:A/Al. This device architecture (illustrated in FIG. 14a) is for long-term stability of OSCs. The procedures of device fabrication were as follows. Firstly, the ITO-coated glass substrates were cleaned with detergent, then ultra-sonicated in acetone and isopropyl alcohol, and subsequently dried in an oven at 100° C. Then, the cleaned ITO substrates were ultraviolet-ozone treated for 20 min to remove tiny organic residues. The zinc oxide (ZnO) solution was prepared using mixture of diethyl zinc solution in toluene and tetrahydrofuran (THF) (1:2, v/v %) and the ZnO film (ca. 35 nm thick) was spin-coated at 4000 rpm for 15 s and annealed at 110° C. for 15 min. Otherwise, ZnO nanoparticles in alcohol (Iso-propanol or/and Methanol) were spin-coated at 3000 rpm for 20 s and annealed at 110° C. for 5 min. The blend solution of P2:NFAs dissolved in organic solvents such as chloroform, chlorobenzene, ortho-dichlorobenzene, etc., which are with and without processing additives such as 1,8-diiodooctane or 1-chloronaphthalene etc. These solutions are spin-coated at 2000 to 4000 rpm for optimization in a nitrogen-filled glove box. The device was pumped down in vacuum (<10⁻⁶ torr), and the MoO₃/Ag (6 nm/100 nm thick) electrode for inverted architecture or A1 (100 nm) electrode for conventional architecture were deposited by thermal evaporation. Photovoltaic characteristics measurements were carried out at the glove box by the solar simulator equipped with a Keithley 2635A source measurement unit. J-V curves were measured under AM 1.5G illumination at 100 mW cm⁻². EQE measurements were conducted in nitrogen-filled glove box using an EQE system.

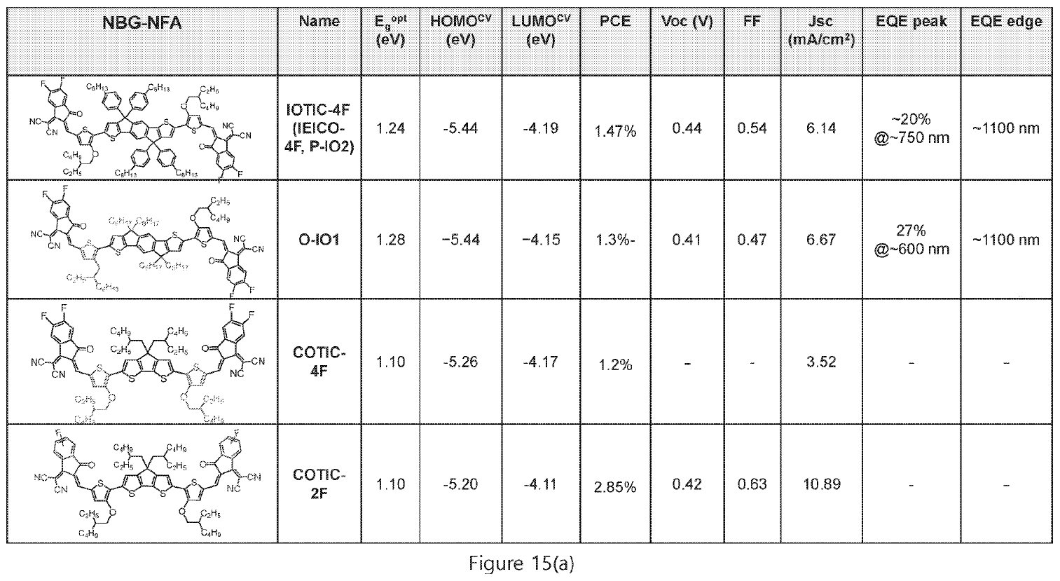

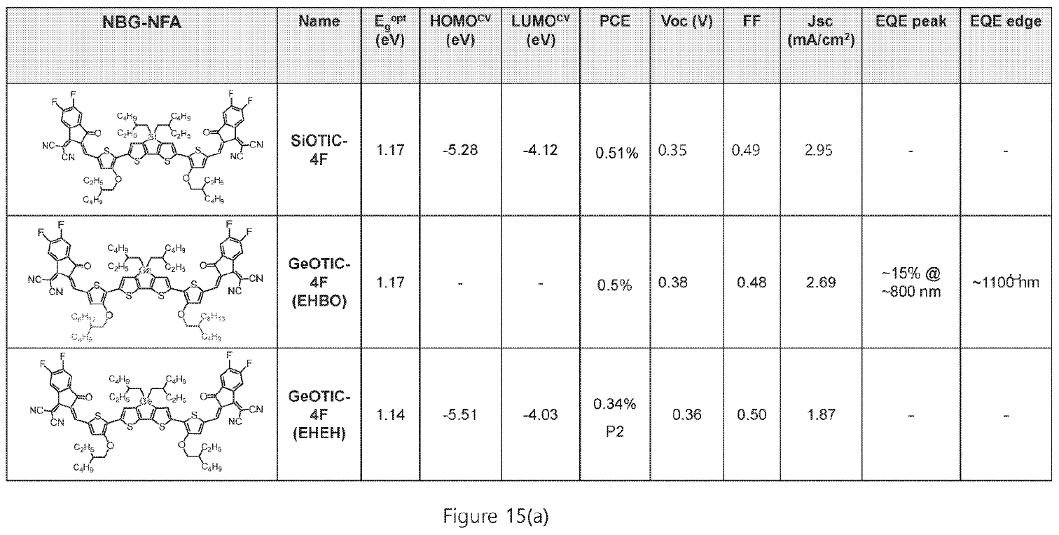

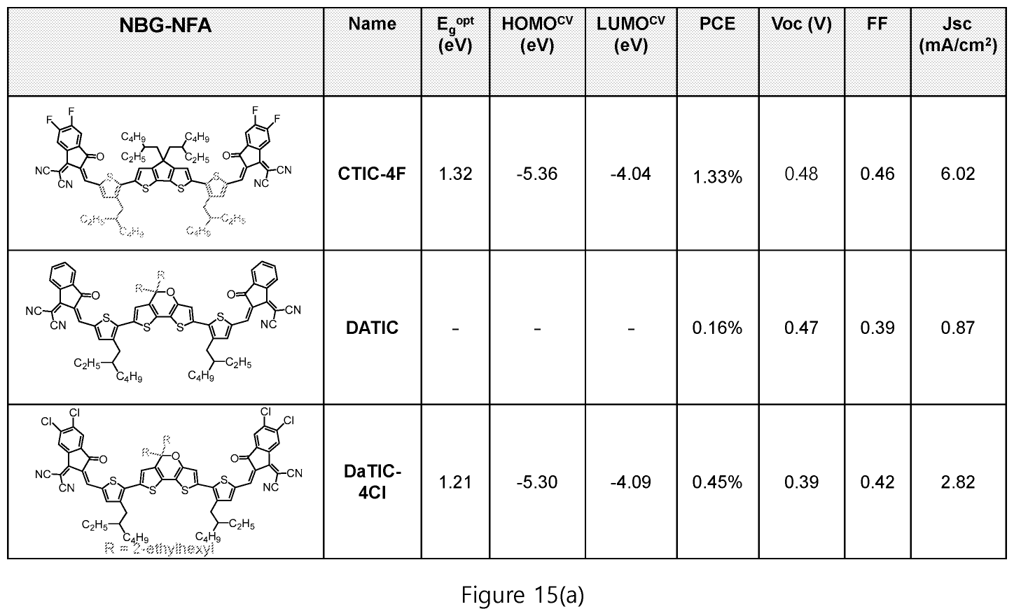

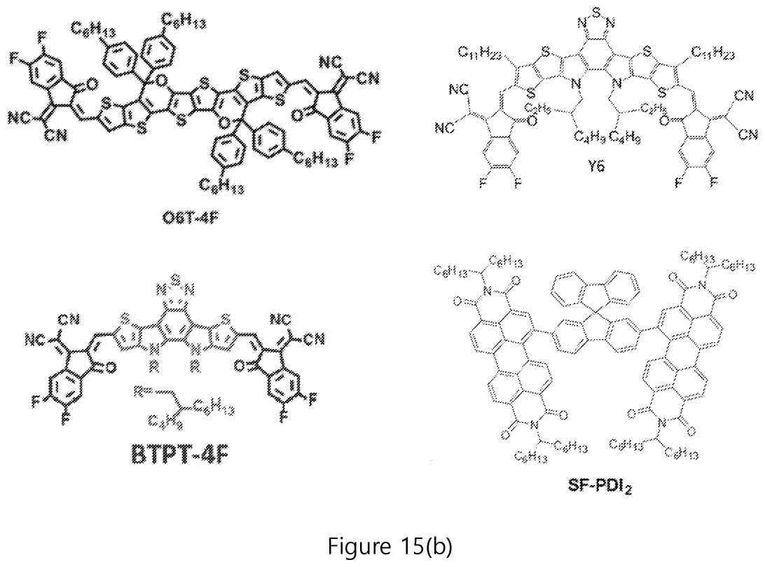

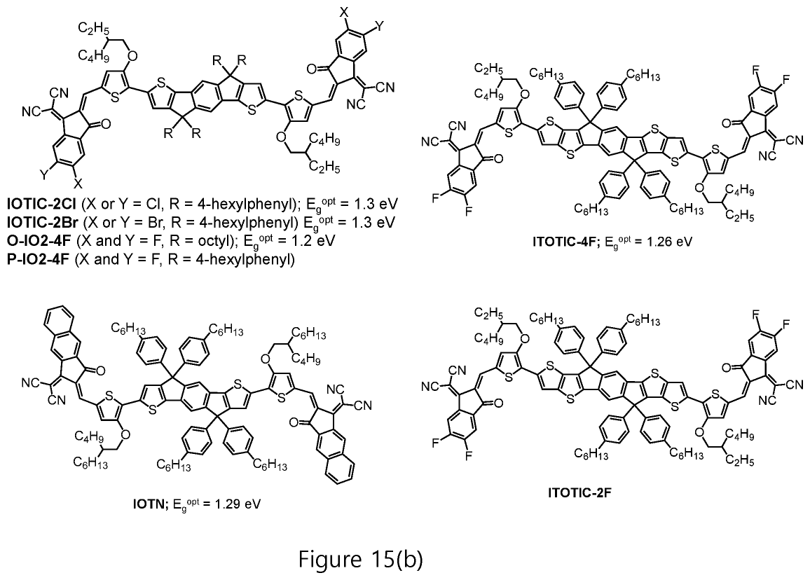

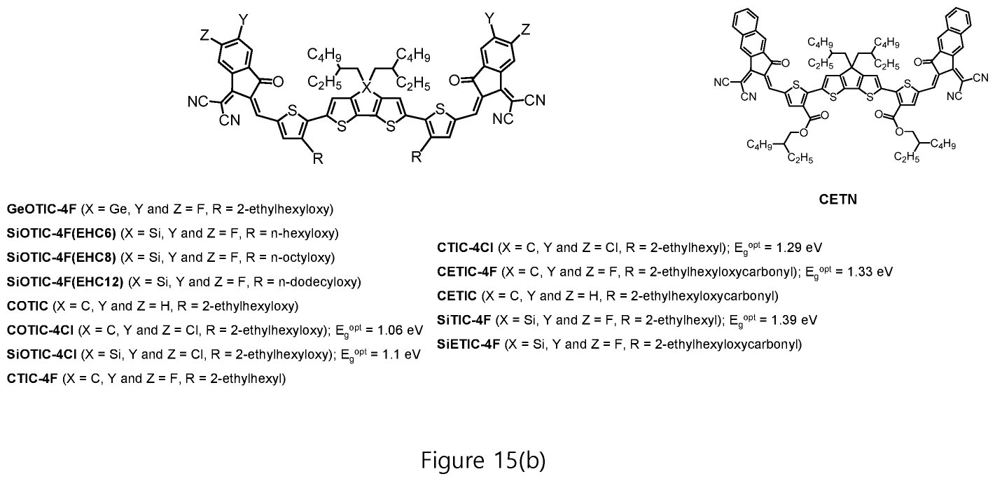

Photovoltaic properties of P2 with various NFAs with bandgap in the range of 1.0-1.3 eV were tested either in a conventional or an inverted device structure. The NFA bandgaps, HOMO-LUMO levels, device V_{oc}, J_{sc}, FFs, PCEs, EQE peaks and EQE edges are summarized in tabular form in FIG. 15(a). Device transmittance spectra, J-V characteristics and EQE spectra are summarized in FIGS. 13(c)-(f), respectively. Surprisingly, the summary demonstrates that P2 as a very narrow bandgap NIR donor works well with various type of very narrow gap NIR non-fullerene acceptors. The devices showed good J_{sc}, V_{OC}, solar cell efficiencies and transparency. EQE maximum up to 30% in the NIR region and EQE responses beyond 1100 nm were demonstrated. Device transmittance over 50% are achieved in the visible light region (400-600 nm). The combination of P2:COTIC-2F exhibited a J_{sc} over 10 mA/cm² and PCE close to 3%, despite of its close to zero HOMO/HOMO energy offset with P2 and very narrow bandgap of 1.1 eV. P2 has a high crystalline packing structure due to its rigid backbone and linear side chain structure, which is expected to give good device thermal stability.

FIG. 13 (h) shows the spectral responsivity of the BHJ photodiodes based on P2:IOTIC-4F, P2:o-IO1 and P2:COTIC-2F, respectively. All devices showed broad photo-response across the visible to NIR region. The maximum responsivity of 0.115, 0.156 and 0.073 A/W was found at 770 nm, 870 nm and 860 nm for P2:IOTIC-4F, P2:o-IO1 and P2:COTIC-2F under short-circuit condition, respectively. The shot noise-limited specific detectivity D* of the photodetectors is shown in FIGS. 13 (j) and (k). All devices showed broad photo-response across the visible to NIR region. At 0 V, high specific detectivity of 1.70×10¹² Jones at 770 nm, 1.93-10¹² Jones at 870 nm, and 6.93×10¹² Jones at 860 nm are obtained for P2:IOTIC-4F, P2: o-IO1 and P2:COTIC-2F based devices, respectively.

### C. Possible Modifications and Variations

As described herein, related non-fullerene acceptor materials can be designed and synthesized with different chemical structures to tune chemical properties for narrower bandgaps and higher device efficiencies. Further examples of NFAs include those illustrated in FIG. 15(b).

In addition, the device performance can be improved through further morphology control of active layer. Further device optimization includes but not limited to donor/acceptor ratio, solvent, solution concentration, processing additive type and amount, film deposition method (spin coating, blade coating, drop casting, spray coating, ink-jet printing etc.), film deposition temperature, film thickness, buffer layers (electron transporting layer, hole transporting layer).

### D. Further Device Examples

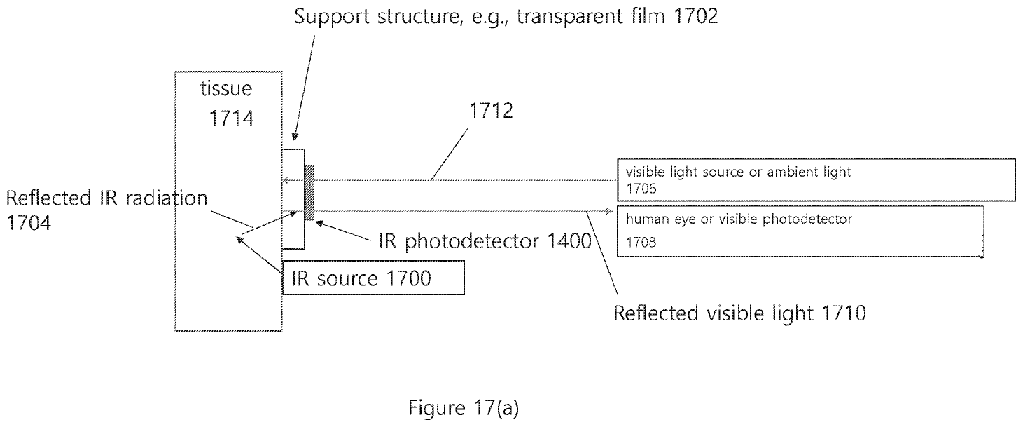

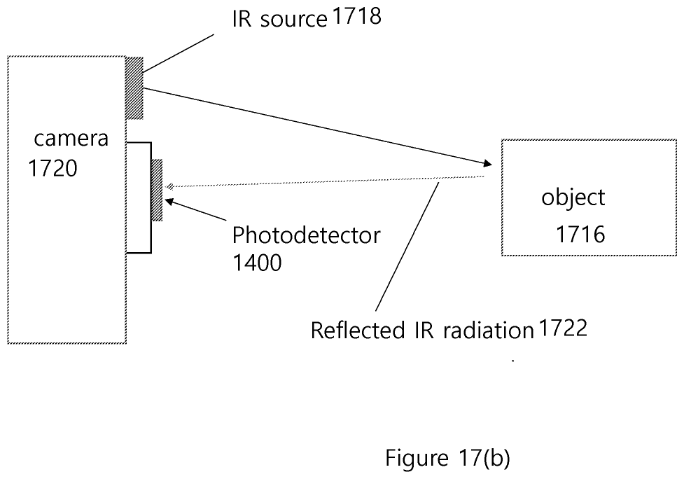

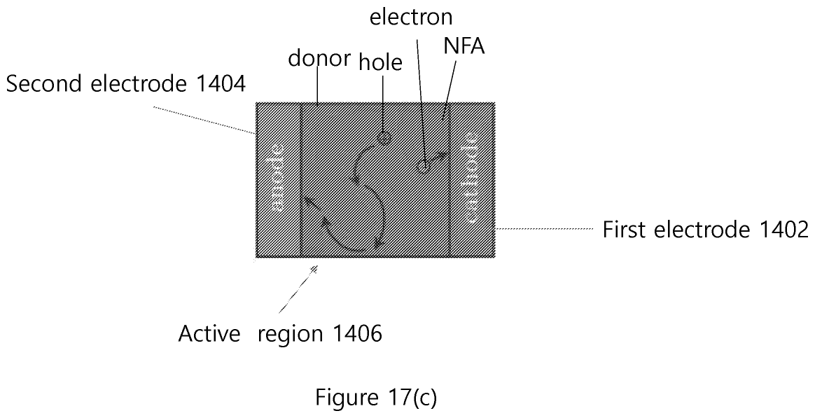

The compositions of matter according to the embodiments described herein may be included in solar cell or photodetector devices having the structure illustrated in FIGS. 14(a)-(b), for example. Although FIGS. 14(a)-(b) are labeled with layers specific compositions, they can be any compositions as described herein. The active region in FIGS. 14(a)-(b) can be the BHJ illustrated in FIG. 17(c).

The above systems can be applied to other organic electronic devices including organic field effect transistor and organic sensor. With a similar working principle to organic solar cells (OSCs), organic photodetectors (OPDs) with NIR responsivity have plenty of applications such as image sensing, night surveillance, optical communication, and health monitoring. Considering that the spectral response window of organic semiconductors can be readily tuned by rational molecular design, NIR OPDs have been emerged as a cost-effective material choice; typically, epitaxial grown inorganic materials such as InGaAs or quantum dots are cost intensive. Narrow bandgap (NBG) non-fullerene electron acceptors (NFA) are an emerging class of NIR organic absorbers that overcome the shortages of the BHJ photodiodes based on fullerenes. Of note are the structural flexibility that provides an opportunity to promote an energy level variability as well as to tailor absorption characteristics toward NIR light with outstanding optoelectronic responses such as efficient charge generation with low photon energy losses. In this context, the recent impressive improvement in device efficiency of OSC is expected to be of particular relevance with the advent of highly efficient NIR NFA materials. Conversely, the vast majority of state-of-the-art OPD systems comprise a narrow bandgap polymer governing the absorption range of the device, combined with a fullerene. These OPDs are expected to exhibit disadvantages over commercially available inorganic devices (e.g. their relatively low photo responsivities in the NIR region), which can be attributed to intrinsic properties of the fullerene acceptor.

As described herein, a bulk heterojunction comprising PM2, P2, X2 or its analogs and an acceptor (NFA) of maximum absorption wavelength in the NIR region generates photo-current under NIR light irradiation while transmitting most of light in the visible region. This unique feature allows us to fabricate semi-transparent NIR photodetectors. These photodetectors can be placed onto a display without greatly changing color tone and reducing brightness so that the display can recognize shape and motion of object in front of it. They can also be placed onto photodetectors detecting visible light without greatly reducing the responsivity of the photodetectors underneath so that the stack of photodetectors can detect visible and NIR separately at the same time. They can be useful not only for displays and image sensors but also for health monitoring systems including blood flow meters, hart rate monitors, and proprioception sensors, and metrology applications. Semi-transparent or transparent photodetectors can open up possibility for easier integration with other optical devices.

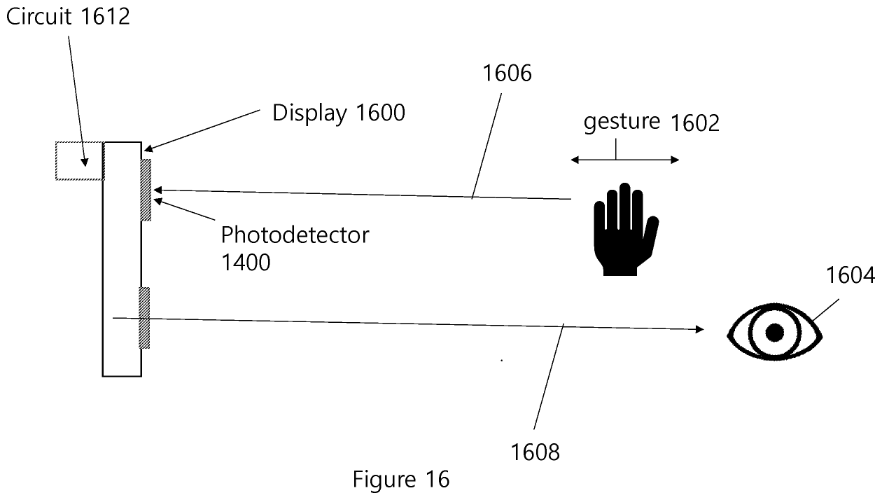

FIG. 16 illustrates a device comprising a photodetector 1400 according to embodiments described herein, comprising a display 1600 emitting electromagnetic radiation 1608 having visible wavelengths (e.g., in a range of 400 nm-600 nm); the photodetector on or above the display; an optional screen or window on or above the photodetector (not shown), wherein the display is readable by eye 1604 of a viewer through the photodetector; and a circuit 1612, computer, or processor connected to the photodetector, the circuit/computer, or processor determining a gesture of the viewer from a signal outputted from photodetector in response to infrared radiation 1606 incident on the photodetector. People (e.g., viewers) can see the images on the monitor or display and the gesture sensor (comprising the photodetectors on the display) can recognize the viewer's gesture(s). In one or more examples, viewers can switch a TV program or channel using their gestures (detected by the photodetector and circuit) without touching the display or a remote control device.

FIG. 17(a) illustrates a health monitoring system (e.g., pulse oximeter) comprising an infrared (IR) light source 1700 and detector 1400 on support structure 1702 (e.g., transparent film). The detector 1400 measures electromagnetic radiation 1704 outputted from the light source and scattered or reflected from living tissue 1714 or cells. The device comprises visible light source 1706 emitting visible electromagnetic radiation 1712. Photodetector 1400 is transparent for visible light 1712 and reflected visible light 1712 so that a doctor or patient may check the condition of the tissue (e.g., the skin condition) under the transparent IR monitoring system (comprising photodetector 1400) using a detector or human eye (1708).

In one or more examples, the health system using NIR photodetectors according to embodiments described herein does not need a separate light source because the detector can sense ambient light, leading to a more compact health monitoring system.

FIG. 17(b) illustrates an example metrology system for measuring system, comprising a camera 1720. In one or more examples, the camera may include the photodetector 1400 according to embodiments described herein. Also shown is infrared source 1718 emitting infrared radiation which is reflected from object 1716 to form reflected infrared (IR) radiation 1722 reflected from the object 1716. Detection of reflected IR radiation 1722 by detector 1400 enables measurement of the distance between camera and the object.

### E. Process Steps

E.1: Example Donors

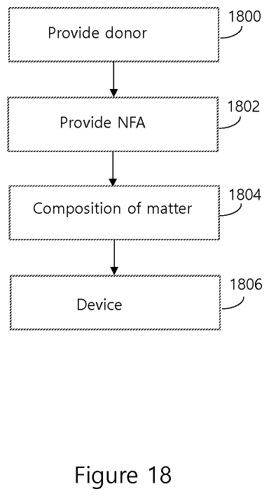

FIG. 18 is a flowchart illustrating a method of making a composition of matter and a device.

Block 1800 represents providing one or more donors (e.g., electron donors).

In one or more examples of this invention, the semiconductor donor compound comprises a conjugated main chain section, the conjugated main chain section having a repeating donor unit (D1) comprises a dithiophene structure.

In one or more embodiments, the repeating donor unit of the semiconductor compound comprises the structure:

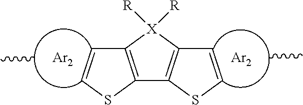

wherein each Ar₂ is independently a substituted or non-substituted aromatic functional group, or each Ar₂ is nothing and the valence of the ring is completed with hydrogen. In some embodiments, the Ar₂ groups can be the same. Each R is independently hydrogen or a substituted or non-substituted alkyl, aryl, alkoxy or thioether chain. In some embodiments, the R groups can be the same. X is C, Si, Ge, N or P. In some embodiments, the R comprising the substituted or non-substituted alkyl, aryl, alkoxy or thioether chain can be a C₆-C₅₀ substituted or non-substituted chain, —(CH₂CH₂O)ₙ (n=2˜20), C₆H₅, —CₙF₍₂ₙ₊₁₎ (n=2˜20), —(CH₂)ₙN(CH₃)₃Br (n=2˜20), 2-ethylhexyl, PhCₘH₂ₘ₊₁ (m=1-20), —(CH₂)ₙN(C₂H₅)₂ (n=2˜20), —(CH₂)ₙSi(CₘH₂ₘ₊₁)₃ (m, n=1 to 20), or —(CH₂)ₙSi(OSi(CₘH₂ₘ₊₁)₃)ₓ(CₚH₂ₚ₊₁)_{y} (m, n, p=1 to 20, x+y=3).

In one or more embodiments, the donor unit comprises:

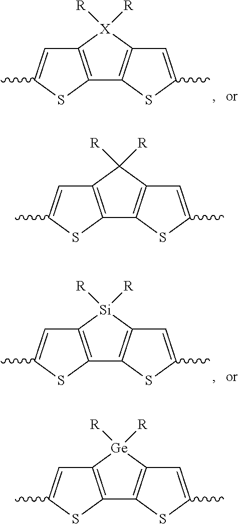

In one or more embodiments, the semiconductor donor compound is a polymer.

In one or more embodiments, the semiconductor compound comprises a conjugated main chain section, the conjugated main chain section having a repeating acceptor unit (A) that comprises the structure:

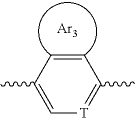

wherein each Ar₃ is independently a substituted or non-substituted aromatic functional group, or each Ar₃ is nothing and the valence of the ring is completed with hydrogen. Each T is N, C—H, C—F or C—Cl.

In one or more embodiments, the semiconductor compound comprises a conjugated main chain section, the conjugated main chain section having an acceptor unit (A) that comprises the structure:

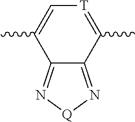

wherein each T is C—H, N, C—F or C—Cl, and Q is O, S, Se or N—R₄, Each R₄ is independently hydrogen or a substituted or non-substituted alkyl or aryl chain.

In one or more embodiments, the acceptor unit comprises:

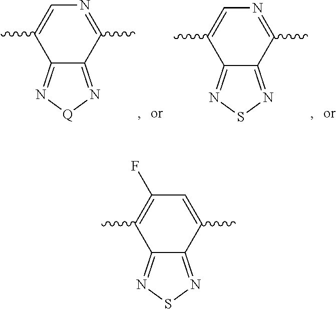

In one or more embodiments, the acceptor unit is regioregularly arranged along the conjugated main chain section.

In one or more embodiments, the semiconductor donor compound is a polymer.

In one or more embodiments, the semiconductor compound comprises a conjugated main chain section, the conjugated main chain section further comprises a second donor unit (D2) that comprises the structure:

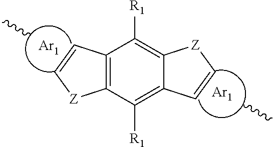

wherein each Ar₁ is independently a substituted or non-substituted aromatic functional group, or each Ar₁ is nothing and the valence of the ring is completed with hydrogen. In some embodiments, the Ar₁ groups can be the same. Each R₁ and R₄ is independently hydrogen or a substituted or non-substituted alkyl, aryl, alkoxy or thioether chain. In some embodiments, the R₁ groups can be the same. Each Z is independently O, S, Se, or N—R₄. In some embodiments, the Z groups can be the same. The R₁ and R₄ comprising the substituted or non-substituted alkyl, aryl, alkoxy or thioether chain can be a C₆-C₅₀ substituted or non-substituted chain, —(CH₂CH₂O)ₙ (n=2˜20), C₆H₅, —CₙF₍₂ₙ₊₁₎ (n=2˜20), —(CH₂)ₙN(CH₃)₃Br (n=2˜20), 2-ethylhexyl, PhCₘH₂ₘ₊₁ (m=1-20), —(CH₂)ₙN(C₂H₅)₂ (n=2˜20), —(CH₂)ₙSi(CₘH₂ₘ₊₁)₃ (m, n=1 to 20), or —(CH₂)ₙSi(OSi(CₘH₂ₘ₊₁)₃)ₓ(CₚH₂ₚ₊₁)_{y} (m, n, p=1 to 20, x+y=3).

In one or more embodiments, the second donor unit comprises:

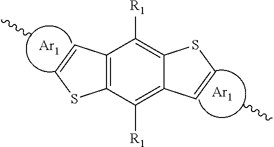

In one or more embodiments, the second donor unit comprises:

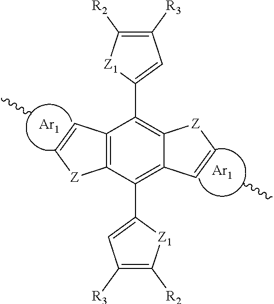

wherein each Ar₁ is independently a substituted or non-substituted aromatic functional group, or each Ar₁ is nothing and the valence of the ring is completed with hydrogen. In some embodiments, the Ar₁ groups can be the same. Each R₂, R₃ and R₄ is independently hydrogen or a substituted or non-substituted alkyl, aryl, alkoxy or thioether chain. In some embodiments, the R₂ groups can be the same. In some embodiments, the R₃ groups can be the same. Each Z and Z₁ is independently O, S, Se, or N—R₄. In some embodiments, the Z groups can be the same. In some embodiments, the Z₁ groups can be the same.

In one or more embodiments, the second donor unit comprises:

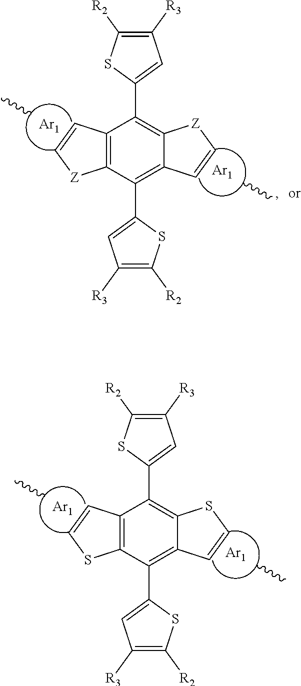

In one or more embodiments, the second donor unit comprises:

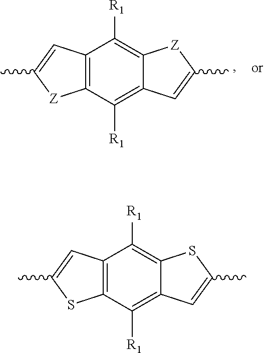

In one or more embodiments, the second donor unit comprises:

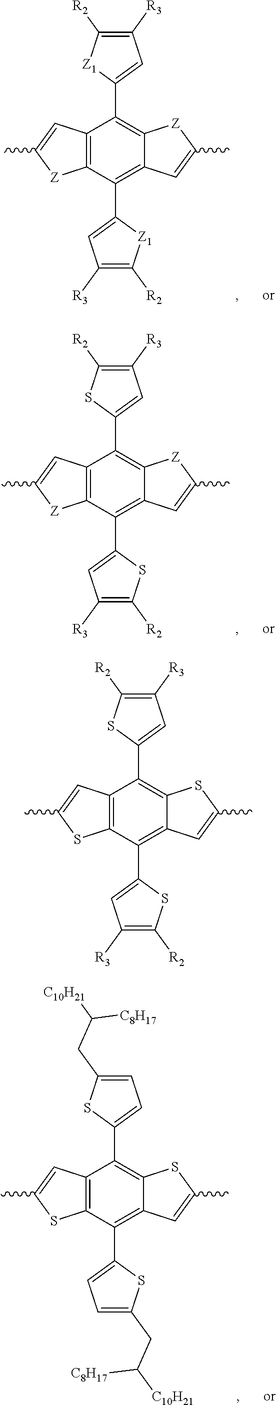

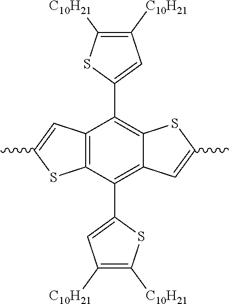

In one or more embodiments, the second donor unit comprises:

In one or more embodiments, the semiconductor donor compound is a polymer.

In one or more embodiments, the semiconductor donor compound comprises a conjugated main chain section, the conjugated main chain section further comprises a second donor unit (D2) that comprises the structure:

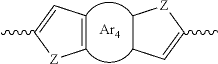

wherein each Ar₄ is independently a substituted or non-substituted aromatic functional group, or each Ar₄ is nothing and the valence of the ring is completed with hydrogen. In some embodiments, the Ar₄ groups can be the same. Each Z is independently O, S, Se, or N—R₄. Each R₄ is independently hydrogen or a substituted or non-substituted alkyl, aryl, alkoxy or thioether chain.

In one or more embodiments, the second donor unit comprises:

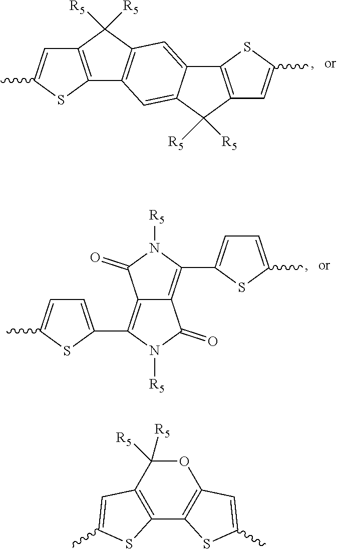

In one or more embodiments, the semiconductor compound is a polymer.

In one or more embodiments, the semiconductor compound comprises a conjugated main chain section, the conjugated main chain section having a D1-A structure, while D1 is a first donor unit, A is an acceptor unit.

In one or more embodiments, the semiconductor compound comprises a conjugated main chain section, the conjugated main chain section having a D1-A structure that comprises the structure:

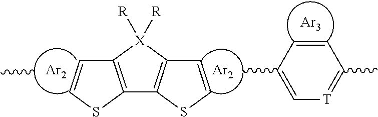

wherein each Ar₂ and Ar₃ is independently a substituted or non-substituted aromatic functional group, or each Ar₂ and Ar₃ is nothing and the valence of the ring is completed with hydrogen. In some embodiments, the Ar₂ groups can be the same. In some embodiments, the Ar₃ groups can be the same. Each R is independently hydrogen or a substituted or non-substituted alkyl, aryl, alkoxy or thioether chain. In some embodiments, the R groups can be the same. X is C, Si, Ge, N or P. T is C—H, N, C—F or C—Cl.

In one or more embodiments, the acceptor unit is regioregularly arranged along the conjugated main chain section.

In one or more embodiments, the conjugated main chain section comprises:

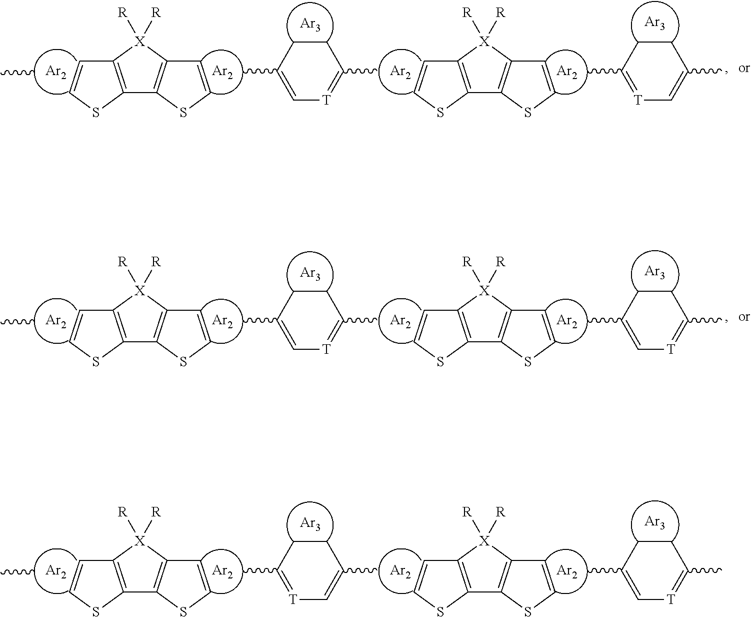

wherein the acceptor unit is regioregularly arranged along the conjugated main chain section as shown in the structure.

In one or more embodiments, the conjugated main chain section comprises:

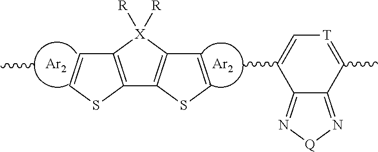

wherein each Ar₂ s independently a substituted or non-substituted aromatic functional group, or each Ar₂ is nothing and the valence of the ring is completed with hydrogen. In some embodiments, the Ar₂ groups can be the same. Each R and R₄ is independently hydrogen or a substituted or non-substituted alkyl, aryl, alkoxy or thioether chain. In some embodiments, the R groups can be the same. X is C, Si, Ge, N or P. T is C—H, N, C—F or C—Cl. Q is O, S, Se or N—R₄.

In one or more embodiments, the conjugated main chain section comprises:

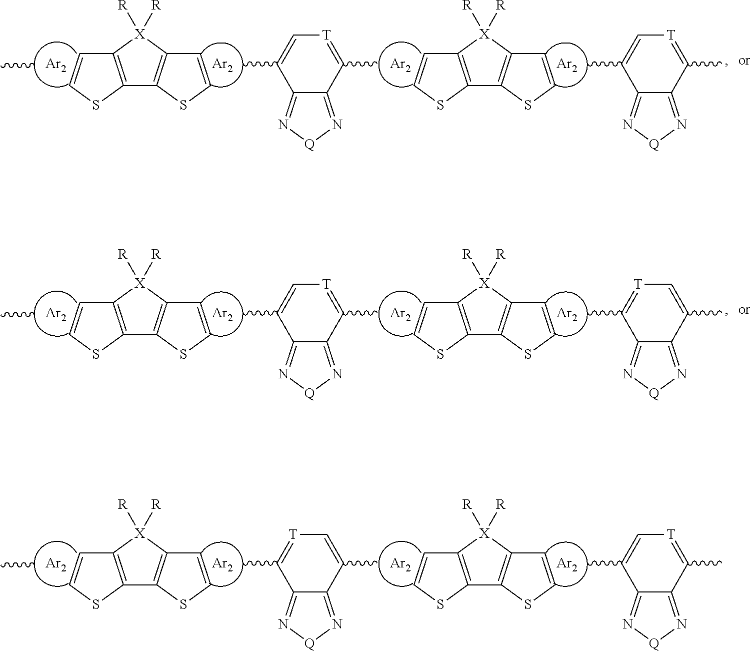

wherein the acceptor unit is regioregularly arranged along the conjugated main chain section as shown in the structure.

In one or more embodiments, the conjugated main chain section comprises;

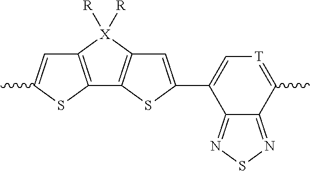

Each R is independently hydrogen or a substituted or non-substituted alkyl, aryl, alkoxy or thioether chain. In some embodiments, the R groups can be the same. X is C, Si, Ge, N or P. T is C—H, N, C—F or C—Cl.

In one or more embodiments, the conjugated main chain section comprises:

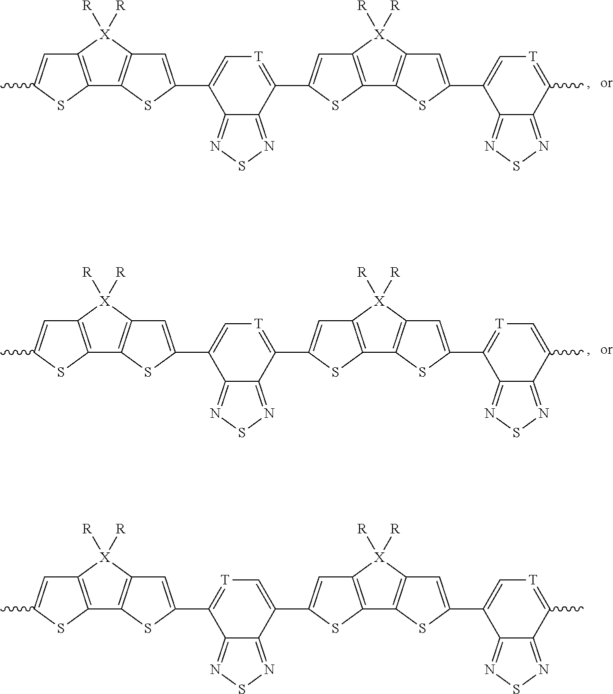

In one or more embodiments, the acceptor unit is regioregularly arranged along the conjugated main chain section.

In one or more embodiments, the conjugated main chain section comprises:

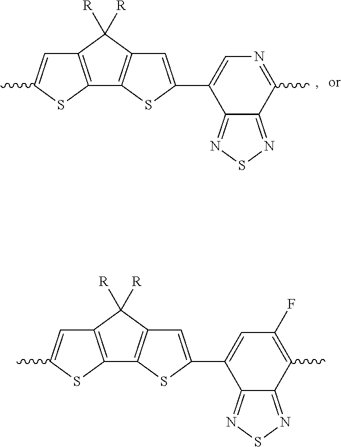

In one or more embodiments, the conjugated main chain section comprises:

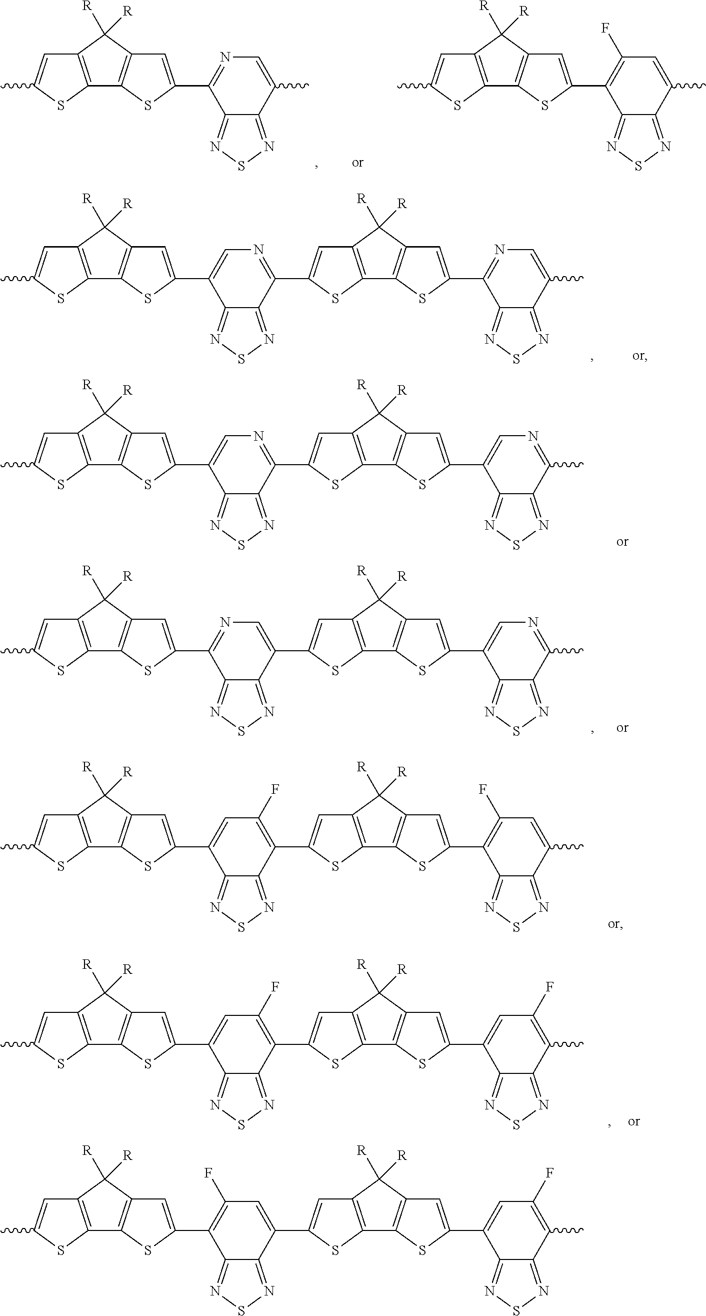

In one or more embodiments, the acceptor unit is regioregularly arranged along the conjugated main chain section.

In one or more embodiments, the semiconductor compound is a semiconductor polymer.

In one or more embodiments, the semiconductor polymer comprises the structure:

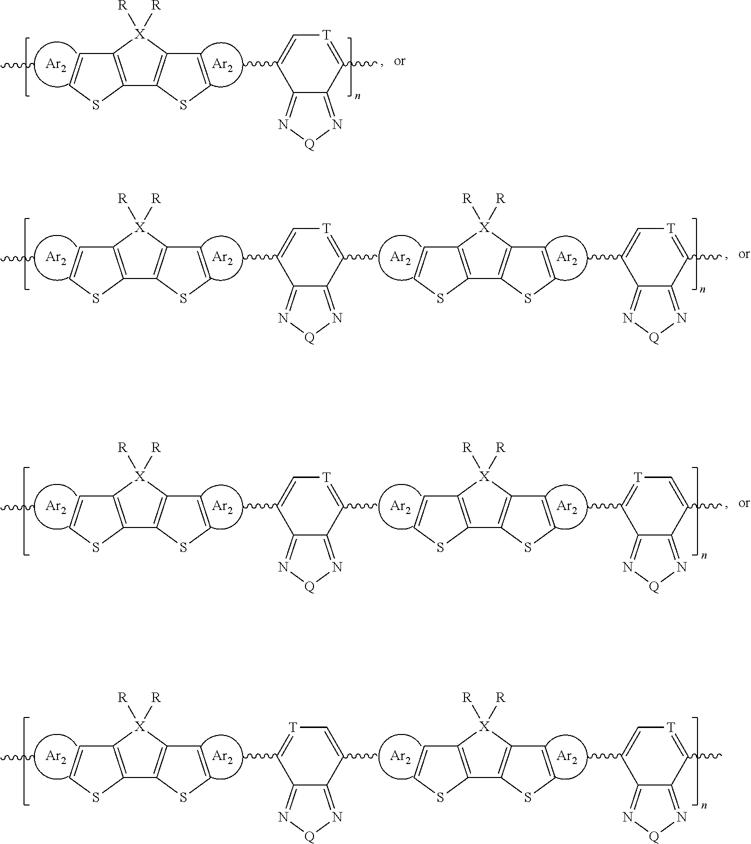

wherein each Ar₂ s independently a substituted or non-substituted aromatic functional group, or each Ar₂ is nothing and the valence of the ring is completed with hydrogen. In some embodiments, the Ar₂ groups can be the same. Each R and R₄ is independently hydrogen or a substituted or non-substituted alkyl, aryl, alkoxy or thioether chain. In some embodiments, the R groups can be the same. X is C, Si, Ge, N or P. T is C—H, N, C—F or C—Cl. Q is O, S, Se or N—R₄. n is an integer.

In one or more embodiments, the acceptor unit is regioregularly arranged along the semiconductor polymer backbone.

In one or more embodiments, the semiconductor polymer comprises the structure:

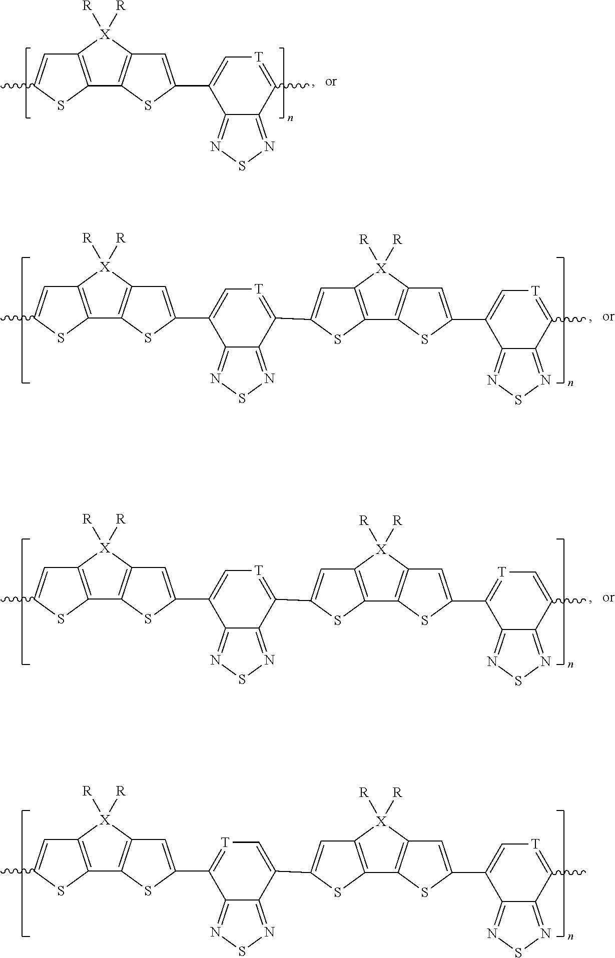

Wherein each R is independently hydrogen or a substituted or non-substituted alkyl, aryl, alkoxy or thioether chain. In some embodiments, the R groups can be the same. X is C, Si, Ge, N or P. T is C—H, N, C—F or C—Cl.

In one or more embodiments, the acceptor unit is regioregularly arranged along the semiconductor polymer backbone.

In one or more embodiments, the semiconductor polymer comprises the structure:

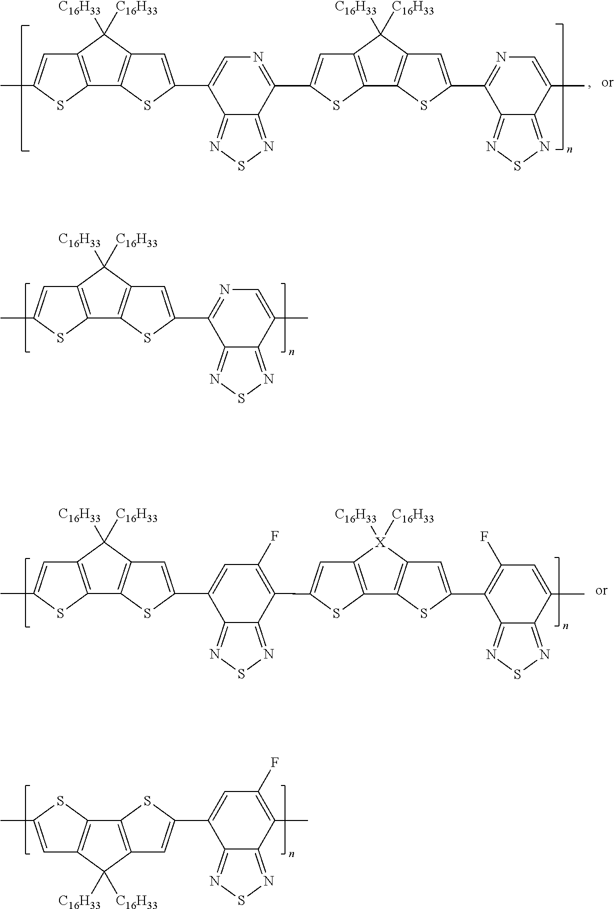

In one or more embodiments, the acceptor unit is regioregularly arranged along the semiconductor polymer backbone.

In one or more embodiments, the semiconducting compound further comprising the structure:

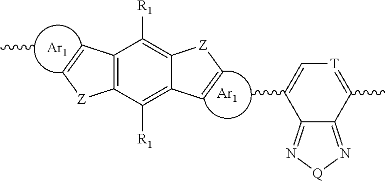

wherein:

each Ar is independently a substituted or non-substituted aromatic functional group, or each Ar is nothing and the valence of the ring is completed with hydrogen. each Z is independently O, S, Se, or N—R₄; each R₁, R₄ is independently hydrogen or a substituted or non-substituted alkyl, aryl, alkoxy or thioether chain. each Q is independently O, S Se, or N—R₄; T is C—H, N, C—F, or C—Cl.

In one or more embodiments, the semiconducting compound further comprising the structure:

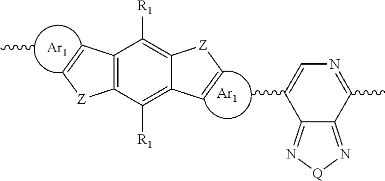

In one or more embodiments, the semiconductor compound comprises a conjugated main chain section, the conjugated main chain section having a D1-A-D2-A structure, while D1 is a first donor unit, D2 is a second donor unit, A is an acceptor unit.

In one or more embodiments, the semiconductor compound comprises a conjugated main chain section, the conjugated main chain section having a D1-A-D2-A structure that comprises the structure:

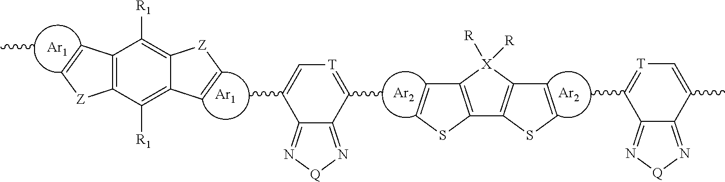

wherein each Ar₁ and Ar₂ is independently a substituted or non-substituted aromatic functional group, or each Ar₁ and Ar₂ is nothing and the valence of the ring is completed with hydrogen. In some embodiments, the Ar₁ groups can be the same. In some embodiments, the Ar₂ groups can be the same. Each R, R₁ and R₄ is independently hydrogen or a substituted or non-substituted alkyl, aryl, alkoxy or thioether chain. In some embodiments, the R groups can be the same. In some embodiments, the R₁ groups can be the same. Each Z is independently O, S, Se, or N—R₄. In some embodiments, the Z groups can be the same. X is C, Si, Ge, Nor P; T is C—H, N, C—F, or C—Cl, and Qis O, S, Se or N—R₄. In one or more embodiments, the acceptor unit is regioregularly arranged along the conjugated main chain section as shown in the structure.

In one or more embodiments, the conjugated main chain section comprises:

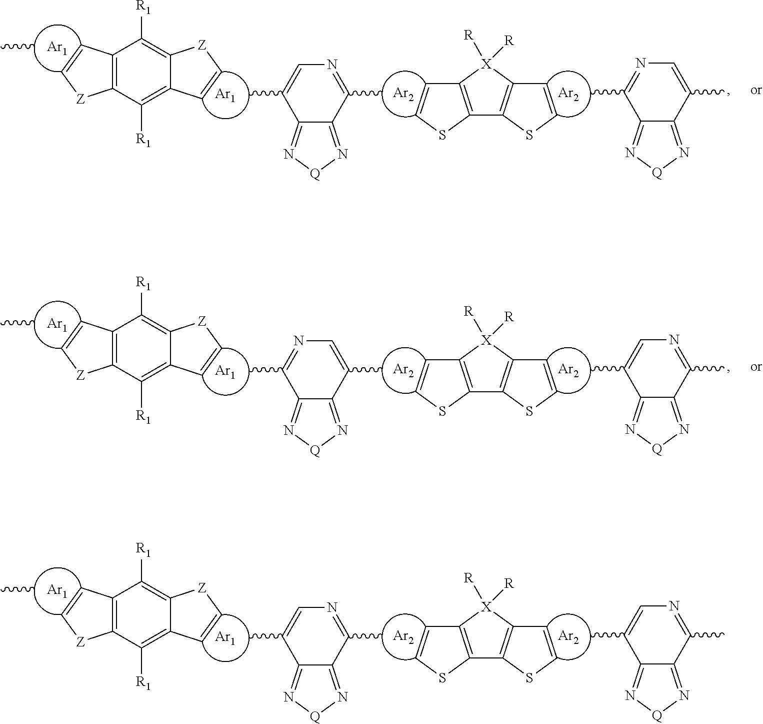

wherein the acceptor unit is regioregularly arranged along the conjugated main chain section as shown in the structure.

In one or more embodiments, the conjugated main chain section comprises:

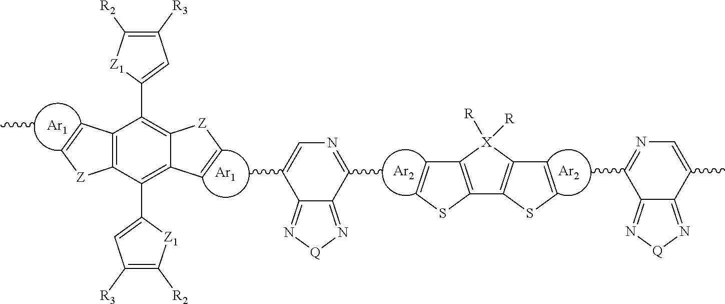

wherein each Ar₁ and Ar₂ is independently a substituted or non-substituted aromatic functional group, or each Ar₁ and Ar₂ is nothing and the valence of the ring is completed with hydrogen. In some embodiments, the Ar₁ groups can be the same. In some embodiments, the Ar₂ groups can be the same. Each R, R₂, R₃ and R₄ is independently hydrogen or a substituted or non-substituted alkyl, aryl, alkoxy or thioether chain. In some embodiments, the R groups can be the same. In some embodiments, the R₂ groups can be the same. In some embodiments, the R₃ groups can be the same. Each Z and Z₁ is independently O, S, Se, or N—R₄. In some embodiments, the Z groups can be the same. In some embodiments, the Z₁ groups can be the same. X is C, Si, Ge, N or P, and Q is O, S, Se or N—R₄. In one or more embodiments, the acceptor unit is regioregularly arranged along the conjugated main chain section as shown in the structure.

In one or more embodiments, the conjugated main chain section comprises:

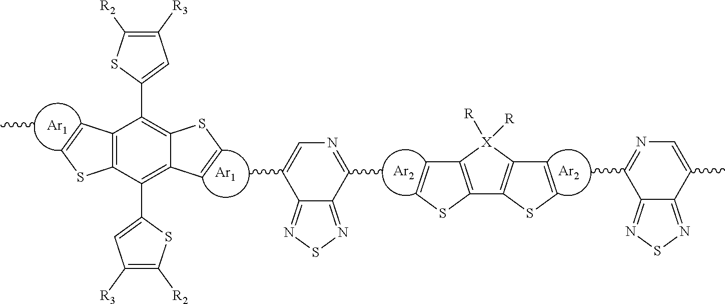

In one or more embodiments, the conjugated main chain section comprises:

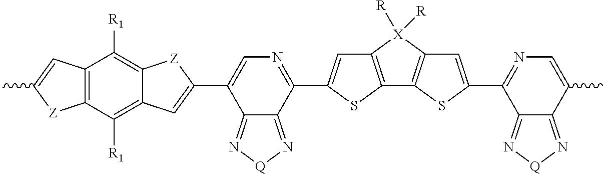

wherein each R, R₁ and R₄ is independently hydrogen or a substituted or non-substituted alkyl, aryl, alkoxy or thioether chain. In some embodiments, the R groups can be the same. In some embodiments, the R₁ groups can be the same. Each Z is independently O, S, Se, or N—R₄. In some embodiments, the Z groups can be the same. X is C, Si, Ge, N or P, and Q is O, S Se, or N—R₄. In one or more embodiments, the acceptor unit is regioregularly arranged along the conjugated main chain section as shown in the structure.

In one or more embodiments, the conjugated main chain section comprises:

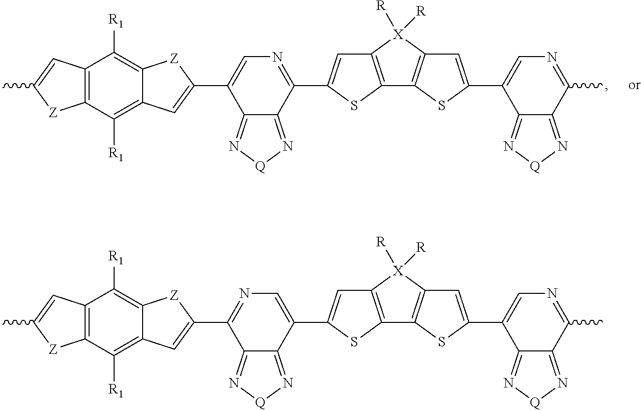

wherein the acceptor unit is regioregularly arranged along the conjugated main chain section as shown in the structure.

In one or more embodiments, the conjugated main chain section comprises:

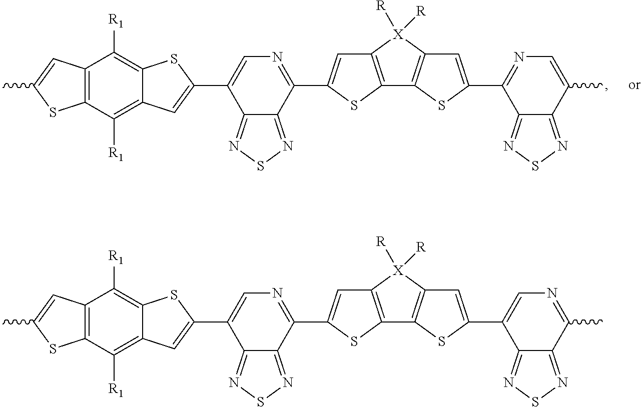

In one or more embodiments, the conjugated main chain section comprises:

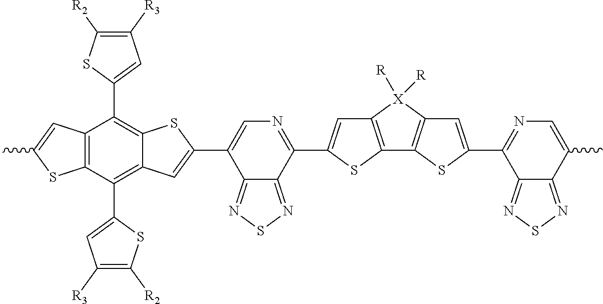

wherein each R, R₂ and R₃ is independently hydrogen or a substituted or non-substituted alkyl, aryl, alkoxy or thioether chain. In some embodiments, the R groups can be the same. In some embodiments, the R₂ groups can be the same. In some embodiments, the R₃ groups can be the same. X is C, Si, Ge, N or P In one or more embodiments, the acceptor unit is regioregularly arranged along the conjugated main chain section as shown in the structure.

In one or more embodiments, the conjugated main chain section comprises.

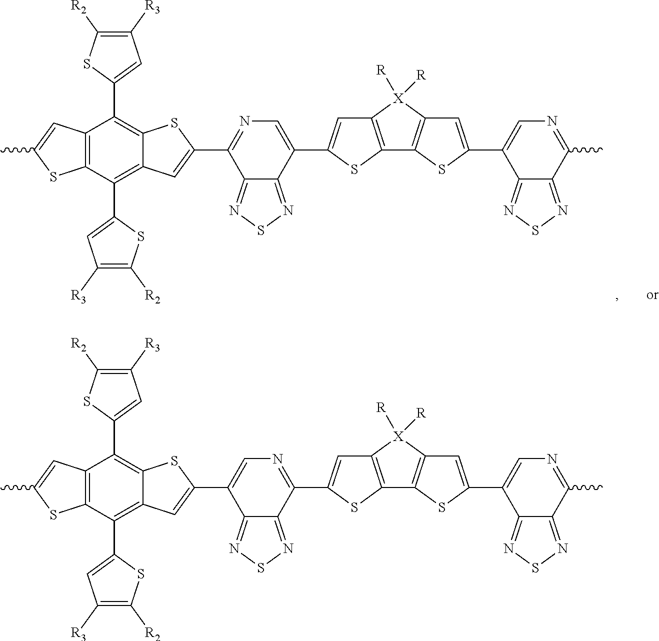

Wherein the acceptor unit is regioregularly arranged along the conjugated main chain section as shown in the structure.

In one or more embodiments, the conjugated main chain section comprises:

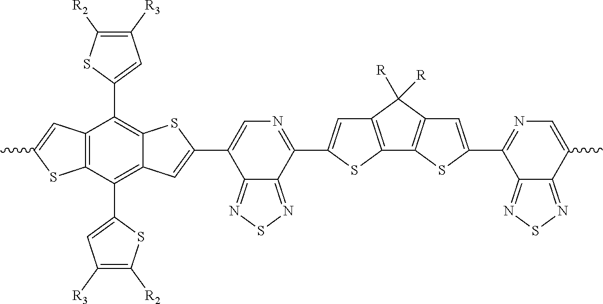

In one or more embodiments, the semiconductor compound is a semiconductor polymer.

In one or more embodiments, the semiconductor polymer comprises:

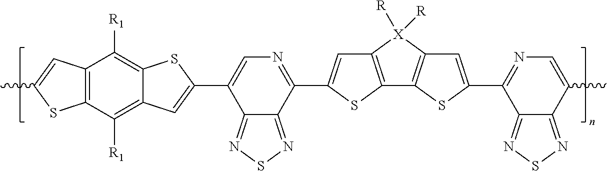

wherein each R and R₁ is independently hydrogen or a substituted or non-substituted alkyl, aryl, alkoxy or thioether chain. In some embodiments, the R groups can be the same. In some embodiments, the R₁ groups can be the same. X is carbon (C), silicon (Si), germanium (Ge), nitrogen (N) or phosphorus (P) In one or more embodiments, the acceptor unit is regioregularly arranged along the conjugated main chain section as shown in the structure.

In one or more embodiments, the semiconductor polymer comprises:

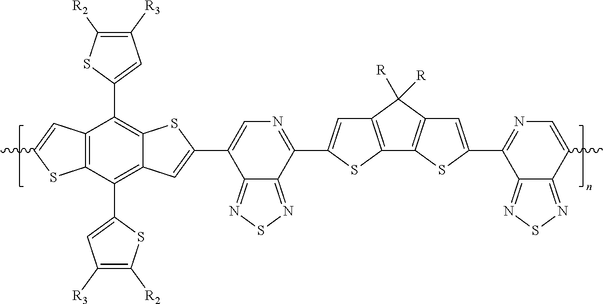

wherein each R, R₂ and R₃ is independently hydrogen or a substituted or non-substituted alkyl, aryl, alkoxy or thioether chain. In some embodiments, the R groups can be the same. In some embodiments, the R₂ groups can be the same. In some embodiments, the R₃ groups can be the same. In one or more embodiments, the acceptor unit is regioregularly arranged along the conjugated main chain section as shown in the structure.

In one or more embodiments, the semiconductor polymer comprises:

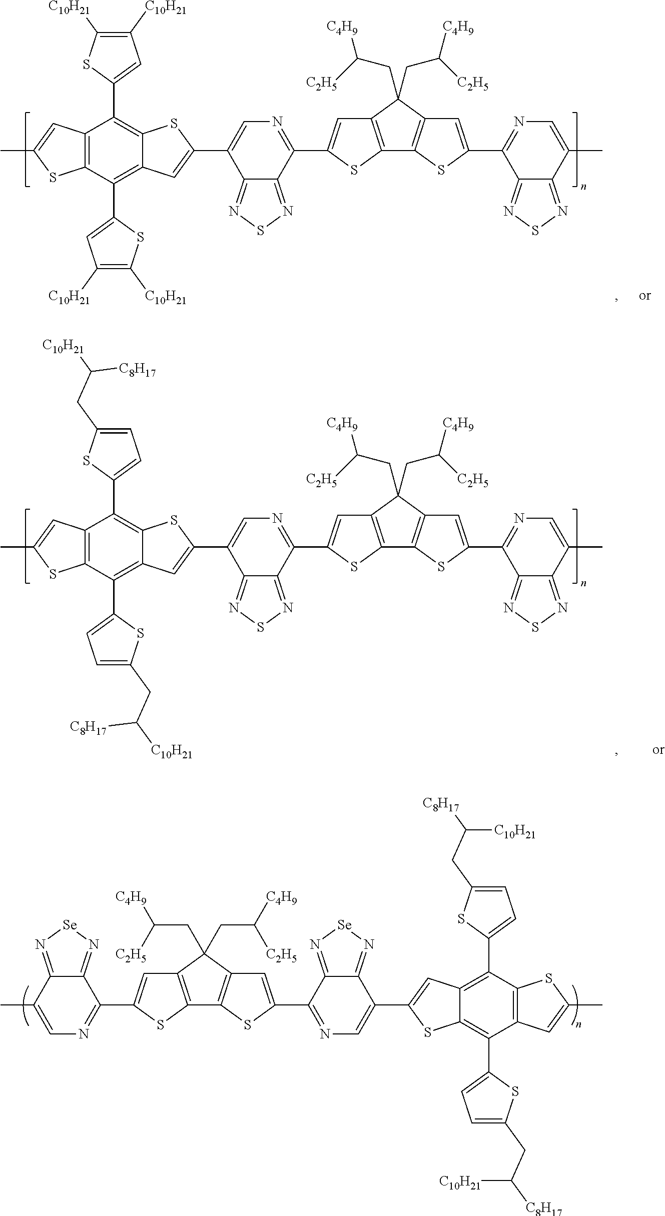

In one or more embodiments, the semiconductor donor polymer comprises a conjugated main chain section, the conjugated main chain section having a repeating unit of D1-A-D2-A structure, while D1 is a first donor unit with strong electron donating capability, D2 is a second donor unit with weaker electron donating capability, A is a strong acceptor unit. Such backbone structures can tune the optical bandgap, HOMO-LUMO levels and yield well-ordered polymer crystallite phases in thin films.

In one or more embodiments, the small molecule donor comprises the structure of E-A-(D1-A)ₙ-E, while D1 can be selected from any of the above listed first donor units, and A can be selected from any of the above listed acceptor units. n=1-10. E is a terminal end group which can be independently hydrogen or a substituted or non-substituted alkyl, aryl or alkoxy group.

In one or more embodiment, the small molecule donor comprises the structure of E-A-(D1-A)ₙ-E, while D1 is

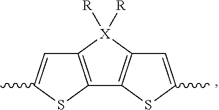

### and A is

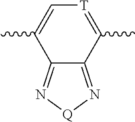

wherein each R and R₄ is independently hydrogen or a substituted or non-substituted alkyl, aryl, alkoxy or thioether chain. X is C, Si, Ge, N or P. T is C—H, N, C—F or C—Cl, and Q is O, S, Se or N—R₄, E is an alkylated bithiophene, n=1, 2, 3, 4 or 5. Each T can be the same or different. Each X can be the same or different.

In one or more embodiment, the small molecule donor comprises the structure:

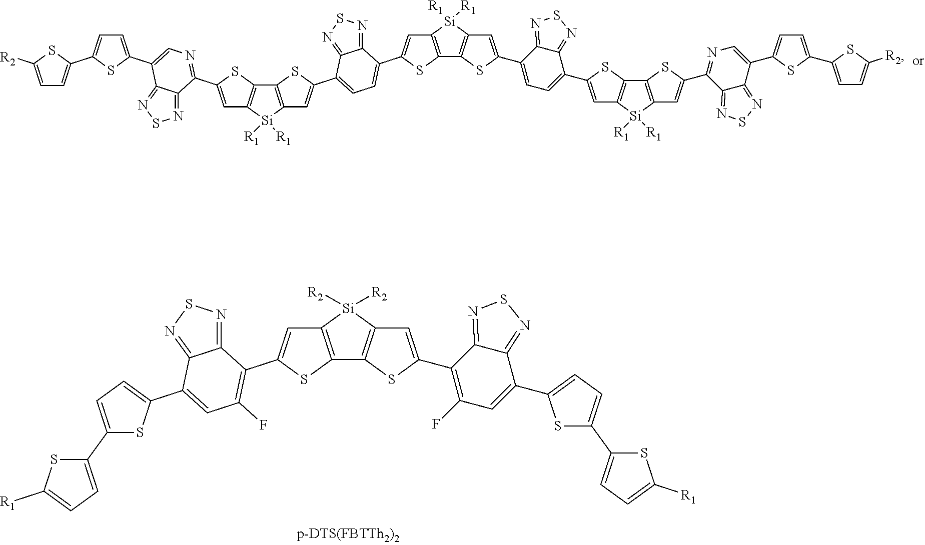

wherein each R₁ and R₂ is independently hydrogen or a substituted or non-substituted alkyl, aryl, alkoxy or thioether chain. In one or more embodiments, R₁ is 2-ethylhexyl, and R₂ is hexyl. In another embodiment, R₂ is 2-ethylhexyl, and R₁ is hexyl.

The R, R₁, R₂, R₃ and R₄ groups can be a linear or branched side-chain comprising a C₃-C₅₀, C₅-C₅₀, C₈-C₅₀, or C₉-C₅₀ substituted or non-substituted alkyl chain. Examples of alkyl chains include isopropyl, sec-butyl, t-butyl, 1,2-dimethylpropyl, 1,1-dimethyl-propyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 6-methylheptyl, 1-methylheptyl, 1,1,3,3-tetramethylbutyl, 1-, 2-, 3-, 4-, 5-, 6- or 7-methyloctyl, 1-, 2-, 3-, 4- or 5-ethylheptyl, 1-, 2- or 3-propylhexyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, dimethyloctyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-, 2-pentylheptyl, branched butyl, pentyl, hexyl, heptyl, octyl, nonyl, decyl, undecyl, dodecyl, tridecyl, tetradecyl, pentadecyl, hexadecyl, heptadecyl, octadecyl, nonoadecyl, eicosyl with one or more branch points at any carbon of the alkyl chain, such as 2 (or 1, or 3, or 4)-ethylhexyl, 2 (or 1, or 3, or 4)-hexyldecyl, 2 (or 1, or 3, or 4)-octyldodecyl, 2 (or 1 or 3, or 4)-butyloctyl, 4 (or 1, or 2, or 3, or 5, or 6)-butyldecyl, 5 (or 1, or 2, or 3, or 4, or 6, or 7)-butylundecyl, 6 (or 1, or 2, or 3, or 4, or 5, or 7, or 8)-butyldodecyl, 12 (or 1, or 2, or 3, or 4, or 5, or 6, or 7, or 8, or 9, or 10, or 11, or 13, or 14)-butyloctadecyl, and the like.

E.2. Example Non-Fullerene Acceptors

Block 1802 represents combining the donor(s) with one or more non-fullerene acceptor (NFA), e.g., to form a blend of the one or more donors and the one or more NFAs.

In one or more embodiments, the non-fullerene acceptors in this invention comprise the general structure:

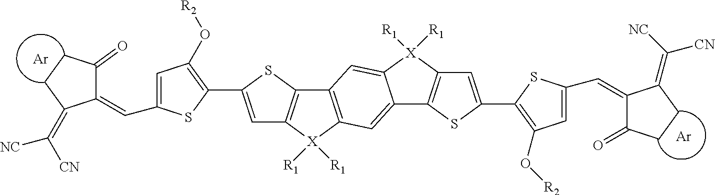

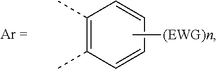

EWG=any electron withdrawing group, can be but not limited to F, Cl. Br, I, CN, CF₃, NO₂, sulfonate, ketone, ester, n=1, 2, 3 or 4. Examples of Ar can be but not limited to the following:

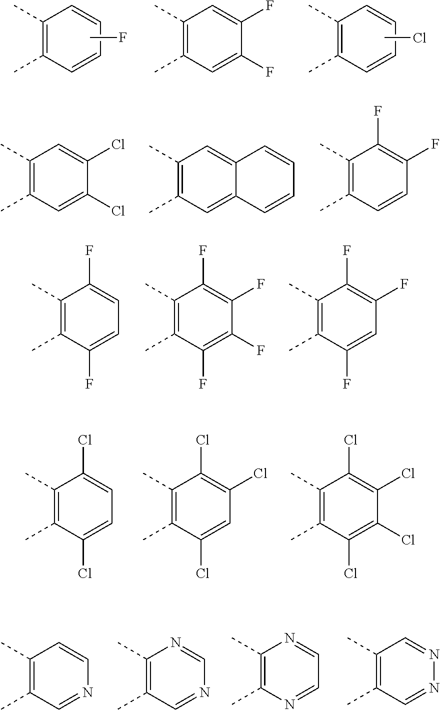

wherein X is C, Si, Ge, N or P; Each R₁ and R₂ is independently hydrogen or a substituted or non-substituted alkyl, aryl or alkoxy chain.

In one or more embodiments, the non-fullerene acceptors in this invention comprise the general structure:

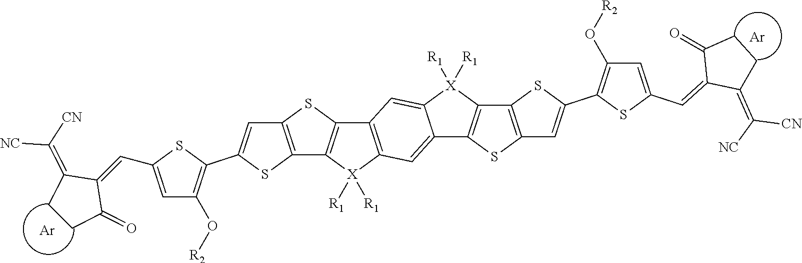

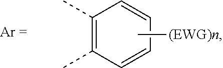

EWG=any electron withdrawing group, can be but not limited to F, Cl. Br, I, CN, CF₃, NO₂, sulfonate, ketone, ester, n=1, 2, 3 or 4. Examples of Ar can be but not limited to the following:

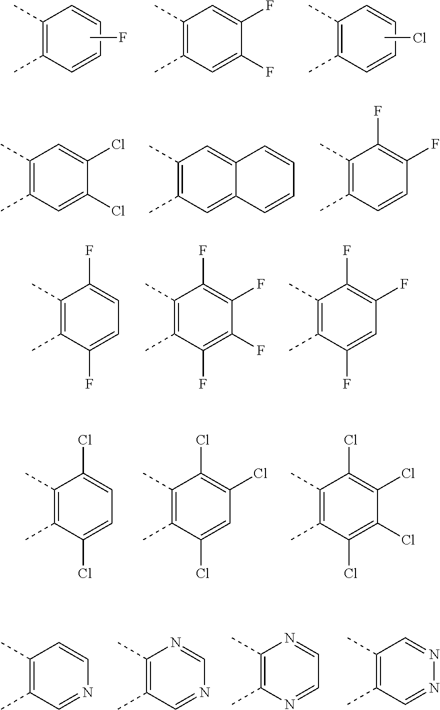

wherein X is C, Si, Ge, N or P; Each R₁ and R₂ is independently hydrogen or a substituted or non-substituted alkyl, aryl or alkoxy chain.

In one or more embodiments, the non-fullerene acceptors in this invention comprise the general structure:

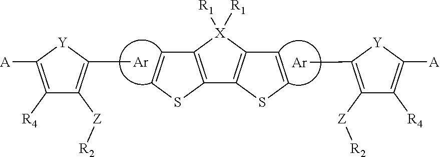

wherein each Ar is independently a substituted or non-substituted aromatic functional group, or each Ar is independently nothing and the valence of its respective thiophene ring is completed with hydrogen, each Ar may comprise one, two, three or more 5-membered or 6-membered aromatic rings; X is C, Si, Ge, N or P; Y is O, S, Se or N—R₃; Z is oxygen (O), sulphur/sulfur (S), selenium (Se), or N—R₃; Each R₁, R₂ and R₃ is independently hydrogen or a substituted or non-substituted alkyl, aryl or alkoxy chain; R₄ is either a hydrogen or the same as Z—R₂. In some embodiments, the R₁, R₂ and R₃ groups can be the same. The R₁, R₂ and R₃ comprising the substituted or non-substituted alkyl, aryl or alkoxy chain can be a C₆-C₅₀ substituted or non-substituted alkyl or alkoxy chain, —(CH₂CH₂O)n (n=2˜30), C₆H₅, —CₙF₍₂ₙ₊₁₎ (n=2˜50), —(CH₂)ₙN(CH₃)₃Br (n=2˜50), 2-ethylhexyl, PhCₘH₂ₘ₊₁ (m=1-50), —(CH₂)ₙN(C₂H₅)₂ (n=2˜50), —(CH₂)ₙSi(CₘH₂ₘ₊₁)₃ (m, n=1 to 50), or —(CH₂)ₙSi(OSi(CₘH₂ₘ₊₁)₃)ₓ(CₚH₂ₚ₊₁)_{y} (m, n, p=1 to 50, x+y=3). Examples of A are listed in FIGS. 22a-g of U.S. patent application Ser. No. 16/179,294.

The R₁, R₂, R₃ and R₄ groups can be a linear or branched side-chain comprising a C₃-C₅₀, C₅-C₅₀, C₈-C₅₀, or C₉-C₅₀ substituted or non-substituted alkyl chain. Examples of alkyl chains include isopropyl, sec-butyl, t-butyl, 1,2-dimethylpropyl, 1,1-dimethyl-propyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 6-methylheptyl, 1-methylheptyl, 1,1,3,3-tetramethylbutyl, 1-, 2-, 3-, 4-, 5-, 6- or 7-methyloctyl, 1-, 2-, 3-, 4- or 5-ethylheptyl, 1-, 2- or 3-propylhexyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, dimethyloctyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-, 2-pentylheptyl, branched butyl, pentyl, hexyl, heptyl, octyl, nonyl, decyl, undecyl, dodecyl, tridecyl, tetradecyl, pentadecyl, hexadecyl, heptadecyl, octadecyl, nonoadecyl, eicosyl with one or more branch points at any carbon of the alkyl chain, such as 2 (or 1, or 3, or 4)-ethylhexyl, 2 (or 1, or 3, or 4)-hexyldecyl, 2 (or 1, or 3, or 4)-octyldodecyl, 2 (or 1 or 3, or 4)-butyloctyl, 4 (or 1, or 2, or 3, or 5, or 6)-butyldecyl, 5 (or 1, or 2, or 3, or 4, or 6, or 7)-butylundecyl, 6 (or 1, or 2, or 3, or 4, or 5, or 7, or 8)-butyldodecyl, 12 (or 1, or 2, or 3, or 4, or 5, or 6, or 7, or 8, or 9, or 10, or 11, or 13, or 14)-butyloctadecyl, and the like.

In one or more embodiments, the non-fullerene acceptors in this invention comprise the general structure:

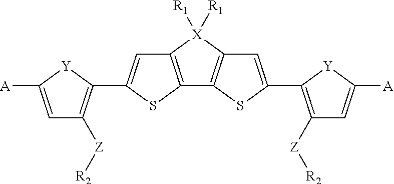

wherein X is C, Si, Ge, N or P; Y is O, S or Se; Z is O or S; Each R₁ and R₂ is independently hydrogen or a substituted or non-substituted alkyl, aryl or alkoxy chain. A is an acceptor moiety. Examples of A are listed in FIGS. 22a-g of U.S. patent application Ser. No. 16/179,294.

In one or more embodiments, the non-fullerene acceptors comprise the general structure:

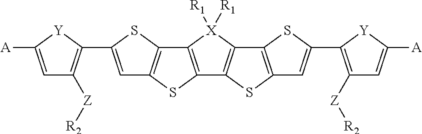

wherein X is C, Si, Ge, N or P; Y is O, S or Se; Z is O or S; Each R₁ and R₂ is independently hydrogen or a substituted or non-substituted alkyl, aryl or alkoxy chain. A is an acceptor moiety. Examples of A are listed in FIGS. 22a-g of U.S. patent application Ser. No. 16/179,294.

In one or more embodiments, the non-fullerene acceptors in this invention comprise the general structure:

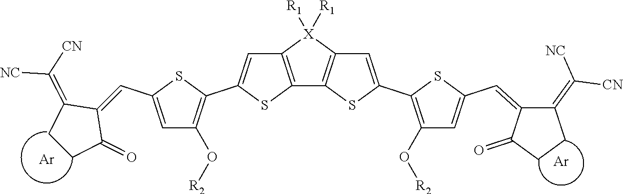

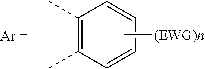

EWG=any electron withdrawing group, can be but not limited to F, Cl. Br, I, CN, CF₃, NO₂, sulfonate, ketone, ester, n=1, 2, 3 or 4. Examples of Ar can be but not limited to the following:

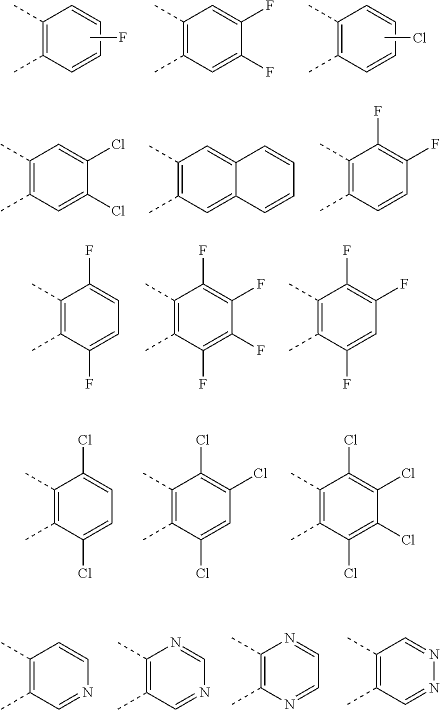

wherein X is C, Si, Ge; Each R₁ and R₂ is independently hydrogen or a substituted or non-substituted alkyl, aryl or alkoxy chain.

In one embodiment, the organic semiconducting molecule has the structure

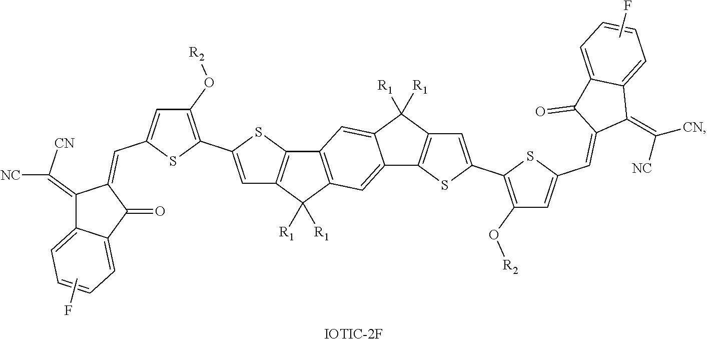

wherein

R₁ is 4-hexylphenyl, and

R₂ is 2-ethylhexyl or R₁ and R₂ are each independently a solubilizing chain, hydrogen or a substituted or non-substituted alkyl, aryl or alkoxy chain as described herein.

In one or more embodiments, the A-D′-D-D′-A semiconductors comprise the general structure:

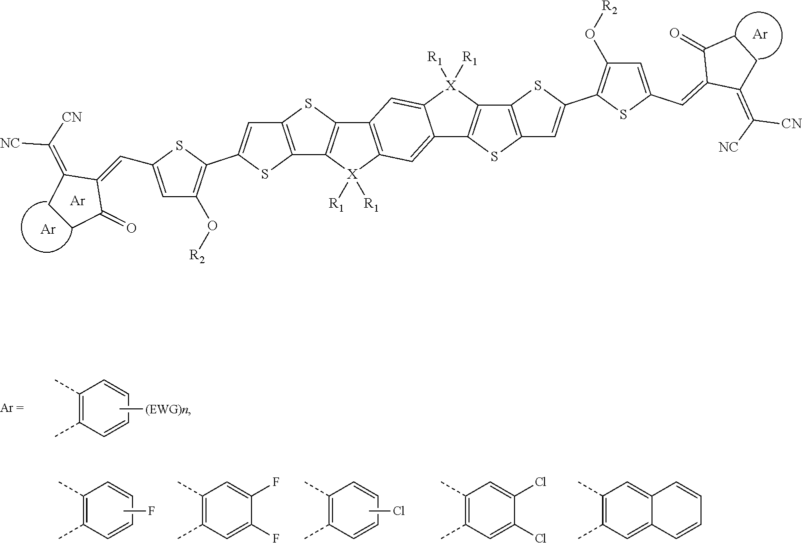

wherein X s C, Si, Ge; EWG=any electron withdrawing group, can be but not limited to F, Cl. Br, I, CN, NO₂, sulfonate, ketone, ester; n=1, 2, 3 or 4; Each R₁ and R₂ is independently hydrogen or a substituted or non-substituted alkyl, aryl or alkoxy chain.

In another embodiment, the NFA has the structure:

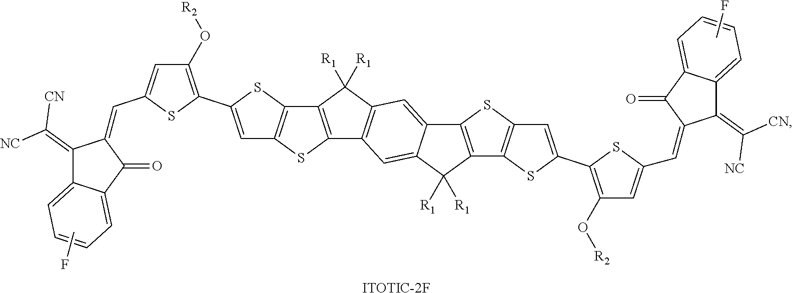

wherein
R₁ is 4-hexylphenyl, and
R₂ is 2-ethylhexyl, or R₁ and R₂ are each independently hydrogen, a solubilizing chain, or a substituted or non-substituted alkyl, aryl or alkoxy chain as described herein.

Other examples include, but are not limited to.

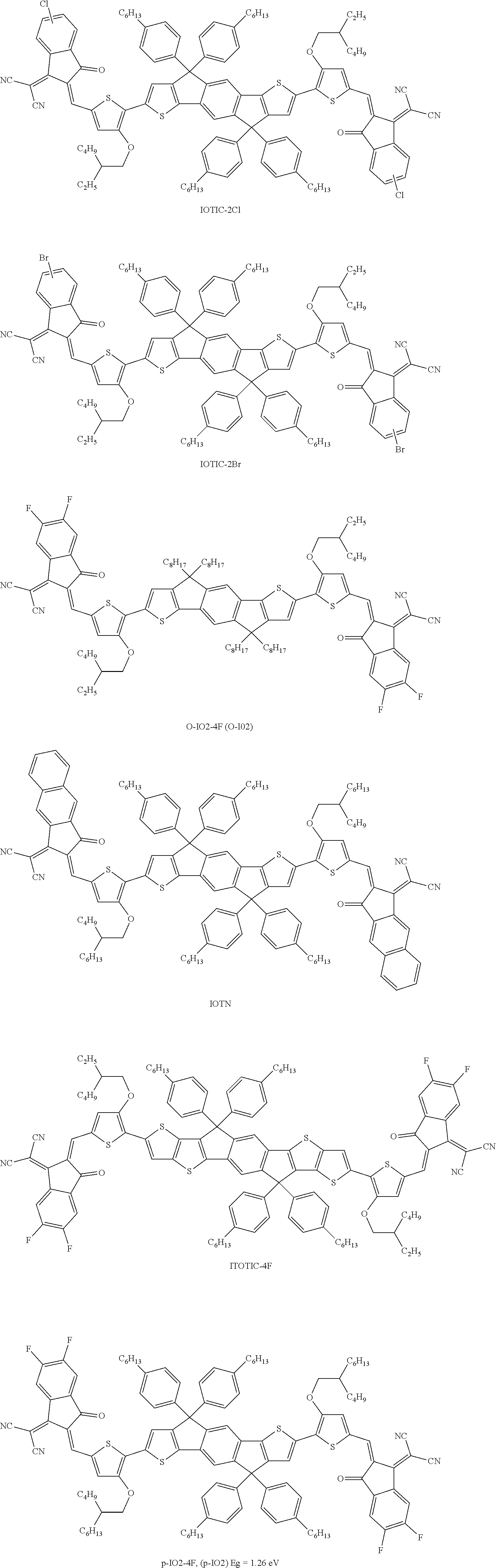

wherein C₈H₁₇, 4-hexylphenyl, C₆H₁₃ and 2-ethylhexyl can be replaced with hydrogen, a solubilizing chain, or a substituted or non-substituted alkyl, aryl or alkoxy chain as described herein.
In yet a further embodiment, the organic semiconducting molecule has the structure:

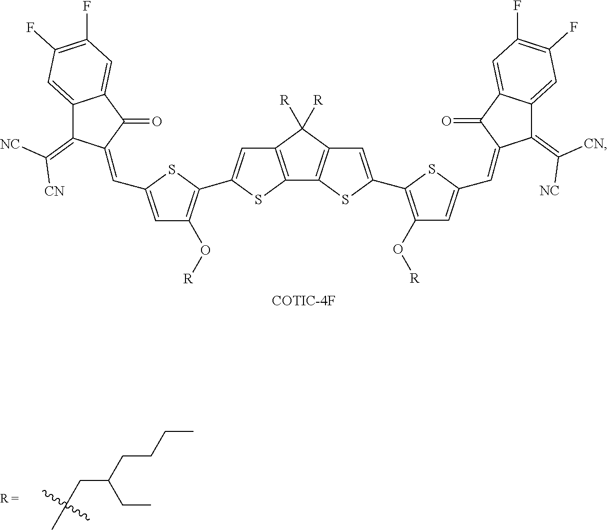

wherein

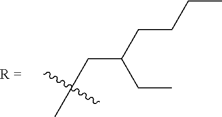 [SMILES: *CC(CC)CCCC]

or R is a solubilizing chain or hydrogen or a substituted or non-substituted alkyl, aryl or alkoxy chain as described herein.

In yet another embodiment, the NFA has the structure:

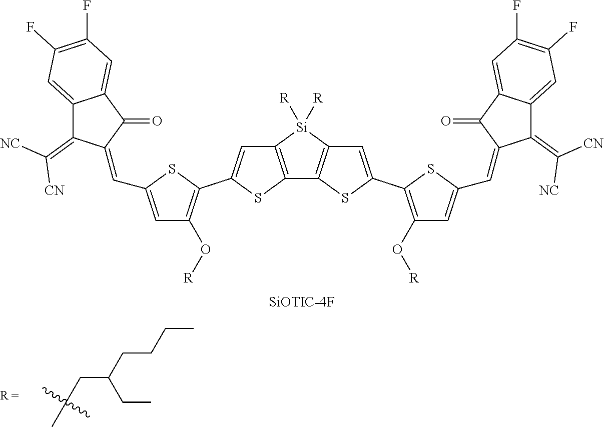

or wherein R is a solubilizing chain or hydrogen or a substituted or non-substituted alkyl, aryl or alkoxy chain as described herein.

In yet further examples, the molecule or acceptor comprises

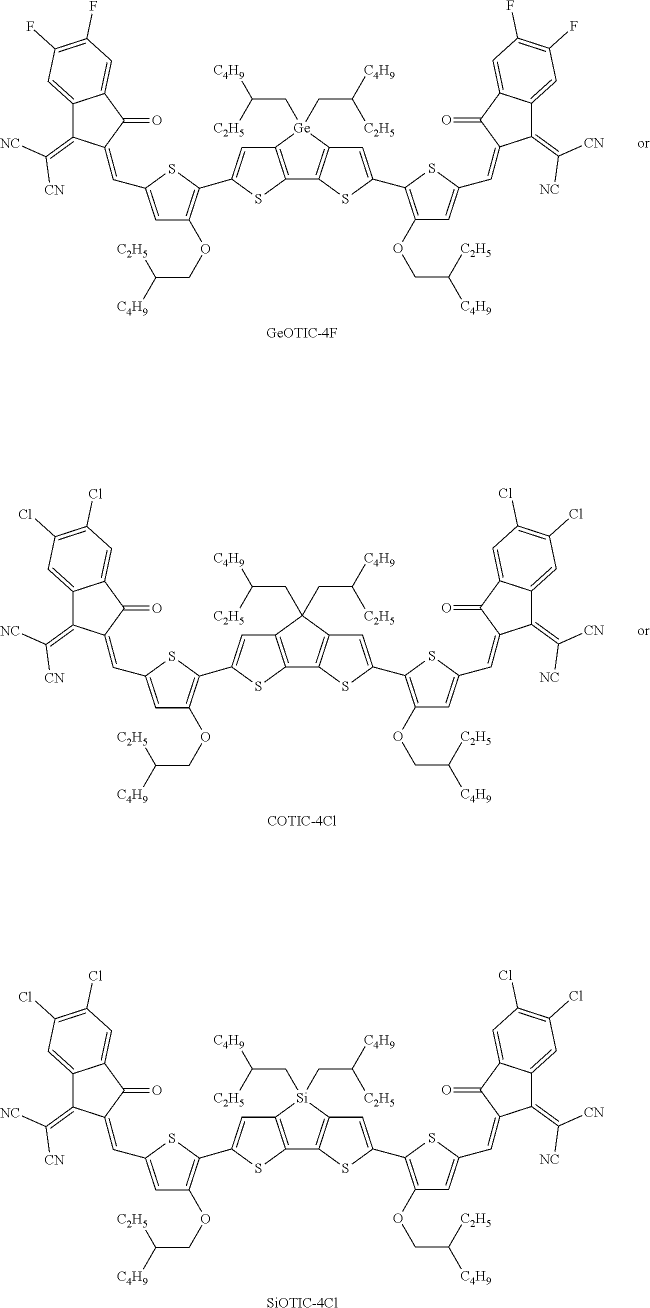

wherein 2-ethylhexyl, C₂H₅, C₄H₉ can be replaced with R that is a solubilizing chain or hydrogen or a substituted or non-substituted alkyl, aryl or alkoxy chain as described herein.

Examples of dithiophene units include those illustrated in Table B (FIG. 30B) in U.S. Utility patent application Ser. No. 14/426,467, filed on Mar. 6, 2015, by Hsing-Rong Tseng, Lei Ying, Ben B. Y. Hsu, Christopher J. Takacs, and Guillermo C. Bazan, entitled “FIELD-EFFECT TRANSISTORS BASED ON MACROSCOPICALLY ORIENTED POLYMERS,” Attorney's Docket No. 30794.0514-US-WO (UC REF 2013-030). Further examples of dithiophene units are illustrated in Table 3 of U.S. Utility patent application Ser. No. 15/406,382, filed on Jan. 1, 2017, by Hsing-Rong Tseng, Lei Ying, Ben B. Y. Hsu, Christopher J. Takacs, and Guillermo C. Bazan, entitled “FIELD-EFFECT TRANSISTORS BASED ON MACROSCOPICALLY ORIENTED POLYMERS,” Attorney's Docket No. 30794.643-US-I1 (UC REF 2013-030-4).

Block 1804 represents the end result, a composition of matter comprising the NFA and the donor.

Block 1806 represents optionally processing the composition of matter in a device.

In one or more examples, the device or composition of matter comprises a plurality of the electron donors and a plurality of the organic semiconducting molecules that are phase separated, wherein the organic semiconducting molecules are disposed in a hierarchical network and the electron donors comprising the second organic semiconducting molecules occupy spaces in the hierarchical network. In one example, the hierarchical network comprises larger mid rib shaped regions connected by smaller or thinner regions. In one example, the composition of matter is solution processed with an additive that promotes formation of the hierarchical network.

In one or more embodiments, the semiconductor donor compound has a HOMO in a range of −4.8 eV to −5.5 eV, a LUMO in a range of −3.5 eV to −4.0 eV, and a bandgap in a range of 1.0 eV to 1.4 eV.

In one or more embodiments, the semiconductor donor compound has HOMO in a range of −5.0 eV to −5.3 eV, a LUMO in a range of −3.5 eV to −3.9 eV, and a bandgap in a range of 1.0 eV to 1.3 eV.

In one or more embodiments, the semiconductor donor compound has a bandgap narrower than 1.3 eV.

In one or more embodiments, the semiconductor donor compound has a bandgap narrower than 1.2 eV or narrower than 1.1 eV.

In one or more embodiments, the semiconductor donor compound has a main absorption band between 600 and 1200 nm.

In one or more embodiments, the semiconductor donor compound has a main absorption band between 600 and 900 nm.

In one or more embodiments, the semiconductor donor compound has a main absorption band between 700 and 1100 nm.

In one or more embodiments, the semiconductor donor compound has a maximum extinction coefficient in solution of at least 1×10 M⁻¹ cm⁻¹.

In one or more embodiments, the NFA has a HOMO in a range of −5.0 eV to −5.5 eV, a LUMO in a range of −3.8 eV to −4.3 eV, and a bandgap in a range of 1.0 eV to 1.4 eV.

In one or more embodiments, the NFA has a bandgap narrower than 1.3 eV.

In one or more embodiments, the NFA has a bandgap narrower than 1.2 eV or narrower than 1.1 eV.

In one or more embodiments, the NFA has a main absorption band between 850 and 1100 nm.

In one or more embodiments, the NFA has a maximum extinction coefficient in solution of at least 1×10⁵ M⁻¹ cm⁻¹.

In one or more embodiments, the device:
Has an active layer thickness less than 3 micrometers, or 1 micrometer;
Has an active layer thickness less than 500 nanometers, or 300 nanometers;
Has an active layer thickness in between 100 nm to 300 nm, or in between 50 nm to 100 nm;
has a transmittance of at least 70% for visible electromagnetic radiation having the wavelength in a range of 400 nm to 600 nm;
has a transmittance of at least 50% for visible electromagnetic radiation having the wavelength in a range of 400 nm to 600 nm;
has an average visible transmittance (AVT) of at least 50% for visible electromagnetic radiation having the wavelength in a range of 370 nm to 750 nm;
has an energetic offset between the donor and acceptor HOMO levels (HOMO_{D}-HOMO_{A}, ΔE_{HOMO}) of no more than 0.2 eV;
has an energetic offset between the donor and acceptor HOMO levels (HOMO_{D}-HOMO_{A}, ΔE_{HOMO}) of no more than 0.1 eV;
has an external quantum efficiency (EQE) over 50%, over 55%, or over 60% in the wavelength range of 600-900 nm; a short circuit current J_{SC} over 17 mA cm⁻²;
has an external quantum efficiency (EQE) over 45%, over 55%, or over 60%, in the wavelength range of 850-1000 nm;
has an external quantum efficiency (EQE) over 10%, over 20%, or over 30%, or over 40% in the wavelength range of 950-1100 nm;
has a short circuit current J_{SC} over 5 mA cm⁻², or over 10 mA cm⁻²;
has a responsivity of 0.5 AW⁻¹ at 920 nm wavelength, −0.1V applied bias.
has a responsivity of 0.35 AW⁻¹ at 900 nm wavelength, 0 V applied bias.
has a responsivity of 0.5 AW⁻¹ at 920 nm wavelength, −2V applied bias.
has a responsivity over 0.3 AW⁻¹ in the wavelength range of 700-900 nm wavelength, 0V applied bias.
has a shot noise-limited specific detectivity over 3×10¹² Jones in the wavelength range of 600-1000 nm wavelength, 0 V applied bias.
has a shot noise-limited specific detectivity over 1×10¹² Jones in the wavelength range of 400-1100 nm wavelength, 0 V applied bias.
has a shot noise-limited specific detectivity over 3×10¹¹ Jones in the wavelength range of 400-1000 nm wavelength, −2.5 V applied bias.
has a dark current as low as 1×10⁻⁹ A/cm² at 0V applied bias.

In one or more embodiments, the active region (e.g., in the solar cell or the photodetector) is sensitive to infrared wavelengths (i.e., the bandgap of the acceptor molecule and/or donor molecule are sufficiently low to absorb infrared radiation).

The photovoltaic device may have a standard or inverted structure. It may comprise a substrate, a first electrode deposited on the substrate, a second electrode, an electron conducting/hole blocking layer deposited either between the first electrode and the active layer, or between the active layer and the second electrode, and an optional hole conducting/electron blocking layer deposited either in between the first electrode and the active layer, or between the active layer and the second electrode.

In one or more examples, the device 1400, as illustrated in FIG. 14, comprises a cathode 1402; an anode 1404; and the active region 1406 having a thickness between the cathode and the anode; and wherein:

holes and electrons are generated in the active region in response to electromagnetic radiation incident on the active region,

the electrons are collected in the electron acceptor and are transmitted through to the cathode, and

the holes are collected in the electron donor and transmitted through to the anode.

Also illustrated is a hole blocking layer 1408 between the cathode and the active region, and an electron blocking layer 1410 between the anode and the active region.

During operation, either or both the electron donor and the electron acceptor absorb photons to create electron-hole pairs, the electron acceptor (interfacing with the electron donor) receives or collects the electron in the electron hole pair and transports the electron to the cathode interface layer/hole blocking layer and the cathode. The hole is transported by the electron donor to the anode interface layer/electron blocking layer and then the anode.

In one or more examples, the device 1400 comprises an infrared photodetector, comprises a first electrode 1402 (e.g., anode or cathode); a first carrier transport layer 1408; an active layer 1406, wherein the first carrier transport layer (e.g., hole blocking layer or electron blocking layer) is between the first electrode and the active layer; a second carrier transport layer 1410, wherein the active layer is between the first carrier transport layer and the second carrier transport layer; and a second electrode 1404 (e.g., anode or cathode) on the second carrier transport layer (e.g., hole blocking layer or electron blocking layer). The active layer comprises the composition of matter of Block 1804 and a transmittance of the photodetector is 50% or more at the wavelengths of 400-600 nm. In one or more embodiments, transmittance of the active layer is 70% or more (e.g., in a range of 70%-90%, 70%-99%, 70%-100%). In one or more examples, transmittance of the photodetector of 50% or more (e.g., in a range of 50%-90%, 50%-99%, 50%-100%) in the wavelengths of 400-600 nm means that the mean transmittance for the wavelengths (in the range of 400-600 nm) is 50% or more. The transmittance of the photodetector can be measured, if the photodetector is stacked on a transparent matter such as window, by differentiating the transmittance of the stack of the transparent matter and the photodetector and that of the transparent matter without the photodetector.

In one or more examples (referring also to FIGS. 14a and 14b), the transmittance of the photodetector is a measure of the amount of the electromagnetic radiation incident on a first surface (e.g., surface A) of the photodetector that is also transmitted through and is outputted through a second surface (e.g., surface B) of the photodetector. In one or more examples, the transmittance may be expressed as a percentage i.e., transmittance is the percentage of the incident intensity I₁ on the first surface that is transmitted as output intensity I₂ through the second surface, or T=100×(I₂/I₁), referring also to FIGS. 14a and 14b.

In one or more examples (referring also to FIGS. 14a and 14b), the transmittance of the active layer is a measure of the amount of the electromagnetic radiation incident on a first surface (e.g., surface C) of the active layer that is also transmitted through and is outputted through a second surface (e.g., surface D) of the active layer 1406 (second surface and first surface on opposite sides of active layer, first surface in contact with first carrier transport layer and second surface in contact with second carrier transport layer). In one or more examples, the transmittance may be expressed as a percentage i.e., transmittance is the percentage of the incident intensity 13 on the first surface of the active layer that is transmitted as output intensity 14 through the second surface of the active layer, or T=100×(I₄/I₃).

In one or more examples, the thickness 1450 of photodetector is preferably less than 3 micrometers.

Examples of a substrate include, but are not limited to, a flexible substrate, a plastic substrate, a polymer substrate, a metal substrate, a silicon substrate, or a glass substrate. In one or more embodiments, the flexible substrate is at least one film or foil selected from a polyimide film, a polyether ether ketone (PEEK) film, a polyethylene terephthalate (PET) film, a polyethylene naphthalate (PEN) film, a polytetrafluoroethylene (PTFE) film, a polyester film, a metal foil, a flexible glass film, and a hybrid glass film. Examples of cathode interface layer include, but are not limited to ZnO and/or ITO. The ZnO can include multiple layers (e.g., two layers) and have a surface roughness of less than 5 nm over an area of 0.2 cm².

Examples of anode interface layer include, but are not limited to MoOx having a thickness in a range of 5-150 nm. Further examples include, but are not limited to, the hole transporting/conducting layer material selected from, but not limited to, the group comprising or consisting of poly(3,4-ethylenedioxythiophene): polystyrene sulfonate (PEDOT:PSS), p-type organic small molecule semiconductors such as Spiro-MeOTAD, pentacene, biscarbazolylbenzene, oligomer semiconductors, polymer semiconductors such as PTAA, poly(3-hexylthiophene-2,5-diyl) (P3HT), donor-acceptor copolymer semiconductors such as PCPDTBT, PCDTBT, metal oxides such as Cul, CuBr, CuSCN, Cu₂O, CuO or CIS. VOₓ, NbOₓ, MoOₓ, WOₓ, NiOₓ, where x is 3 or less than 3, or other main group or transition metal oxides and a compound as shown in FIG. 1 of U.S. Ser. No. 14/954,131.

Examples of cathode material include, but are not limited to, ITO. In further examples, the electron transporting/conducting layer material is selected from, but not limited to, the group comprising or consisting of TiO₂, ZnO, SnO, SnO₂, SiO₂, CeO₂, ZrO₂, CdSe, WO₃, ZnSnO₄, PbI₂, SrTiO₃, fullerene based electron acceptors (C₆₀, C₇₀, PC₆₁BM, PC₇₁BM, ICBA), borane based electron acceptors (3TPYMB), Bathocuproine (BCP), bathophenanthroline (Bphen), ITIC type of non-fullerene acceptors, NDI and PDI based non-fullerene acceptors, and the combination of above (double layer). The electron transporting layer may have a thickness of 2 nm to 500 nm, preferably a thickness of 20 nm to 200 nm, more preferably a thickness of 20-50 nm, or 50 nm to 100 nm.

Examples of cathode and anode materials include, but are not limited to, a metal or at least one material selected from gold, aluminum, copper, silver, silver paste, palladium, platinum, nickel, a combination/bilayer of metal and molybdenum oxide or molybdenum (wherein the MoOx is an interlayer), a liquid metal (e.g., mercury alloy, eutectic gallium indium), a transparent conductive layer, carbon nanotubes, graphene, carbon paste, PEDOT:PSS, and a conjugated polvelectrolyte.

In one or more embodiments, the electrodes, anode, cathode, interface layers, electron transporting/hole blocking layers, hole transporting/electron blocking layers of the electronic device can be transparent or semitransparent.

The active layer, electron transporting/hole blocking layers, hole transporting/electron blocking layers of the electronic device may be deposited by solution casting or vapor deposition. Illustrative thin film deposition methods include a spin coating method, a casting method, a microgravure coating method, a gravure coating method, a bar coating method, a roll coating method, a blade coating method, a wire bar coating method, a dip coating method, a spray coating method, a free span coating method, a dye coating method, a screen printing method, a flexo printing method, an offset printing method, an inkjet printing method, a dispenser printing method, a nozzle coating method and a capillary coating method, for forming a film from a solution.

In one or more examples, the active layer has a thickness in a range of 50-600 nm. In some embodiments, the active layer has a thickness in a range of 87-300 nm. In some embodiments, the active layer has a thickness of at least 300 nanometers or in a range of 200 nm to 1 micrometer.

### F. Advantages and Improvements

Advantages of organic photosensitive devices include the ability to tune optical and electrical properties for NIR irradiation absorption. Small molecules and polymers which absorb up to NIR wavelengths (Absorption >700 nm) have been reported with improvement of OPV performance due to the enhanced current density from electrical conversion of photon in NIR region.^{[11-13]} Introduction of non-fullerene acceptors has also led to improved performance of organic solar cells beyond the limit of PCBM used in the past.^{[8-10]}

However, conventional organic photovoltaic (OPV) devices face some competition with silicon solar cells and perovskite solar cells in terms of commercial applications as a kind of next generation energy source due to their relatively lower device efficiencies. The present disclosure has surprisingly demonstrated that organic devices according to embodiments described herein can include active region compositions with frontier molecular orbitals and molecular structure tailored for strong absorption profiles (and high device efficiency) in the NIR while maintaining high transparency at visible wavelengths. Such significantly improved transparency is surprising because materials that are more transparent at visible wavelengths are typically also be more transparent at near infrared wavelengths, leading to less desirable photosensitive properties at the near infrared wavelengths. Specifically, is not easy to develop narrow gap donors and acceptors, and it is much harder to develop a combination with good absorptive performance at NIR wavelengths when the bandgaps become narrower and narrower. When the bandgaps become narrower, it is more difficult to achieve the energy offset between HOMO_{D}/HOMO_{A} and the energy offset between LUMO_{D}/LUMO_{A} (see FIG. 1(b)) needed to achieve charge separation of electrons and holes for proper functioning of the solar cell or photodetector device (HOMO_{D} is the HOMO of the donor, HOMO_{A} the HOMO of the acceptor, LUMO_{D} is the LUMO of the donor, and LUMO_{A} is the LUMO of the acceptor, where HOMO is Highest occupied molecule orbital and LUMO is the lowest un-occupied molecular orbital).

Moreover, the HOMOs and LUMOs are affected by the molecular conformation (flat, bent etc.) and the environment (crystalline or amorphous domains, surrounded by other donors or acceptors etc.). Thus, in a BHJ the HOMOs and LUMOs are a range instead of a single number. As a result, it is harder to develop new materials that have the right HOMO/LUMO range and energy offset when the bandgaps become narrower and narrower.

The improved transmission of the active regions at visible wavelengths has enabled new devices (e.g., displays with gesture sensors or pulse oximeters) requiring higher transparency at visible wavelengths.

Device and Composition of Matter Examples

Compositions of matter and devices according to embodiments described herein include, but are not limited to, the following.

2. A composition of matter, comprising:

a semiconducting compound of the structure (and isomers thereof):

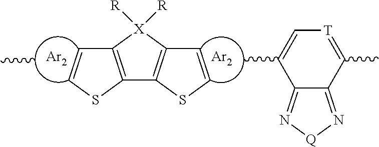

wherein:

each Ar₂ is independently a substituted or non-substituted aromatic functional group, or Ar₂ is nothing and the valence of the ring is completed with hydrogen;

each Q is independently O, S Se, or N—R₄;

each R and R₄ is independently hydrogen or a substituted or non-substituted alkyl, aryl, alkoxy or thioether chain;

T is N, C—F, or C—Cl;

X is C, Si, Ge, N or P; and

a non-fullerene acceptor combined with the semiconducting compound.

2. The composition of matter of example 1, wherein the semiconducting compound comprises the structure:

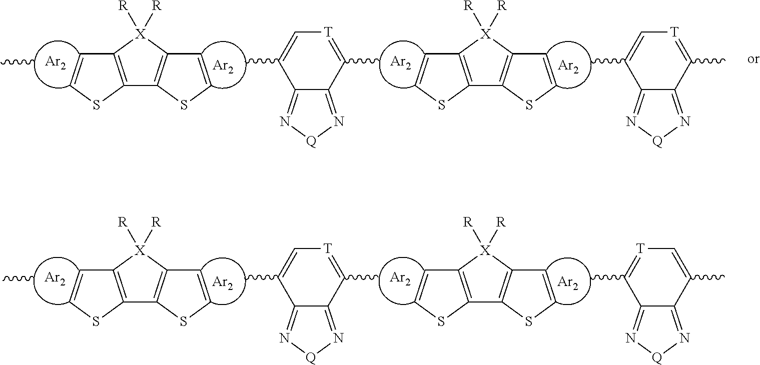

3. The composition of matter of example 2, wherein the semiconducting compound comprises the structure:

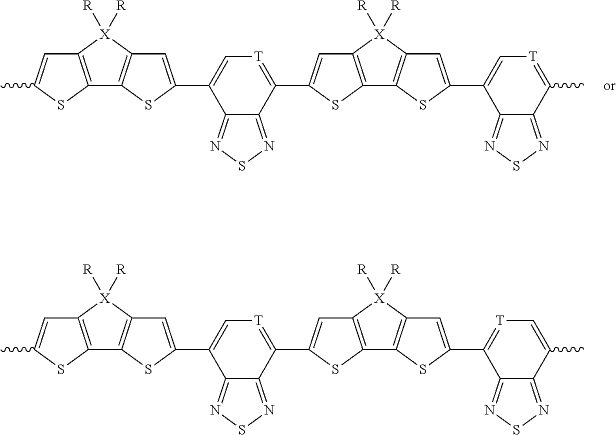

4. The composition of matter of example 1, wherein the semiconducting compound comprises the structure:

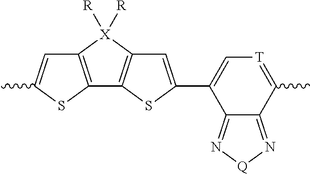

wherein:

each R is independently hydrogen or a substituted or non-substituted alkyl, aryl, alkoxy or thioether chain;

T is N, C—F, or C—Cl; and

X is C, Si, Ge, Nor P.

5. The composition of matter of example 1, wherein the semiconducting compound further comprises the structure:

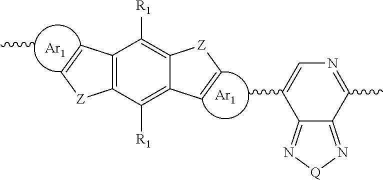

wherein:

each Ar₁ is independently a substituted or non-substituted aromatic functional group, or each Ar₁ is nothing and the valence of the ring is completed with hydrogen.

each Z is independently O, S, Se, or N—R₄;

each R₁, R₄ is independently hydrogen or a substituted or non-substituted alkyl, aryl, alkoxy or thioether chain.

each Q is independently O, S Se, or N—R₄,

6. The composition of matter of example 5, wherein the semiconducting compound comprises the structure:

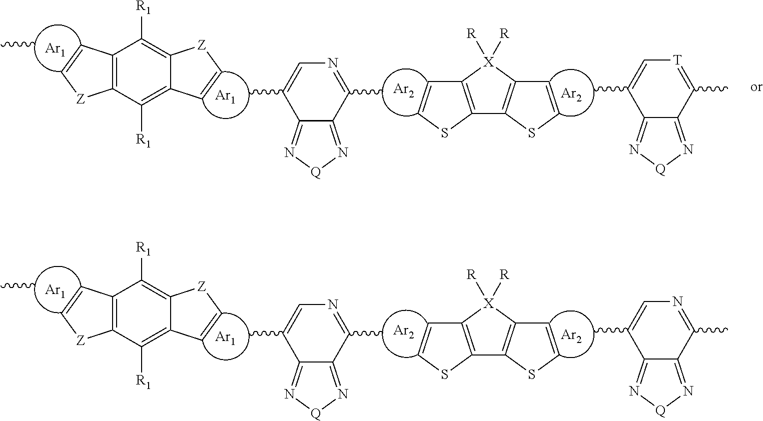

7. The composition of matter of example 6, wherein the semiconducting compound comprises the structure:

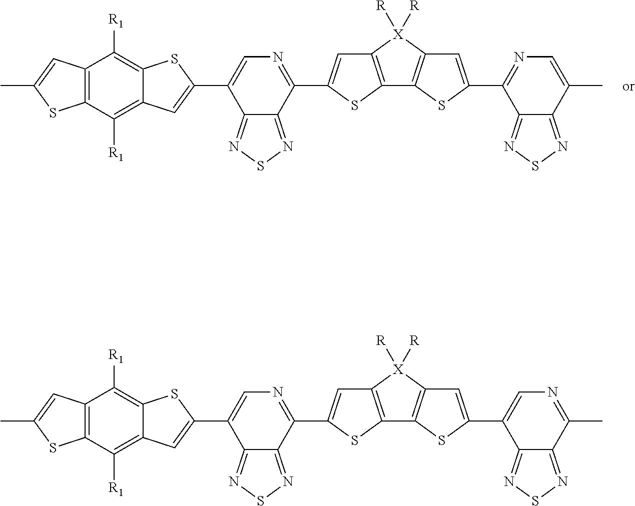

8. The composition of matter of any of the examples 1-7, wherein the non-fullerene acceptor has the structure:

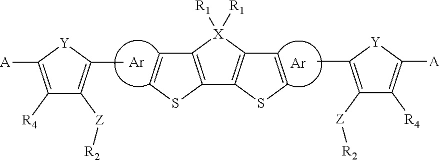

wherein:

each Ar is independently a substituted or non-substituted aromatic functional group, or each Ar is independently nothing and the valence of its respective thiophene ring is completed with hydrogen;

X is C, Si, Ge, N or P; Y is O, S, Se or N—R₃;

Z is O, S, Se, or N—R₃;

A is an acceptor moiety; and

each R₁, R₂ and R₃ is independently hydrogen or a substituted or non-substituted alkyl, aryl or alkoxy chain: R₄ is either a hydrogen or the same as Z—R₂.

9. The composition of matter of any of the examples 1-8, wherein the non-fullerene acceptor has a bandgap less than or equal to the bandgap of the semiconducting compound.

10. The composition of matter of any of the examples 1-9, wherein the semiconducting compound comprises a semiconducting polymer having a repeat unit comprising the structure of any of the examples 1-7.

11. The composition of matter of example 10, wherein the semiconducting polymer comprises a semiconducting polymer having the structure:

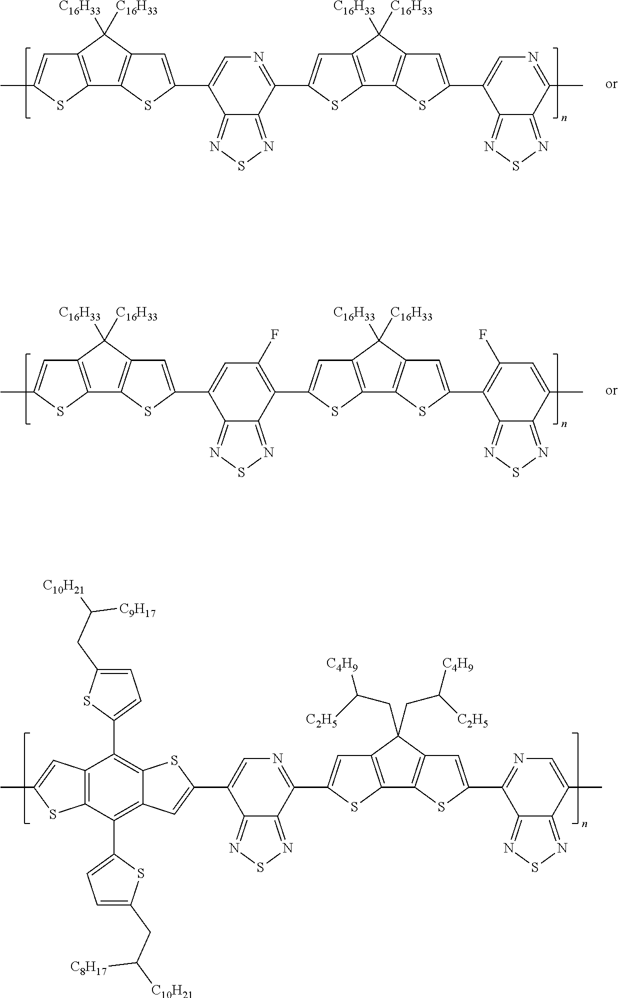

12. The composition of matter of examples 10 or 11, wherein the acceptor unit in the semiconducting compound is regioregularly arranged along the conjugated main chain section (the side chains comprising C and H, e.g., C₁₀H₂₁ can be any R comprising a substituted or non-substituted alkyl, aryl or alkoxy chain as described herein.

13. The composition of matter of any of the examples 1-12, wherein the non-fullerene acceptor comprises;

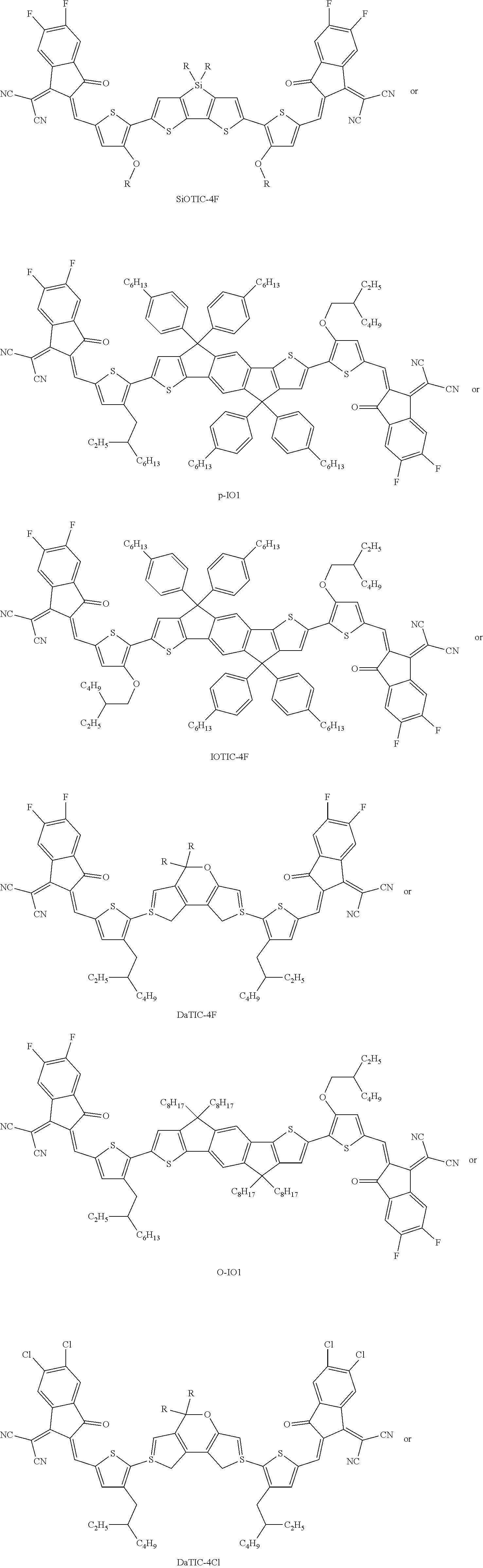

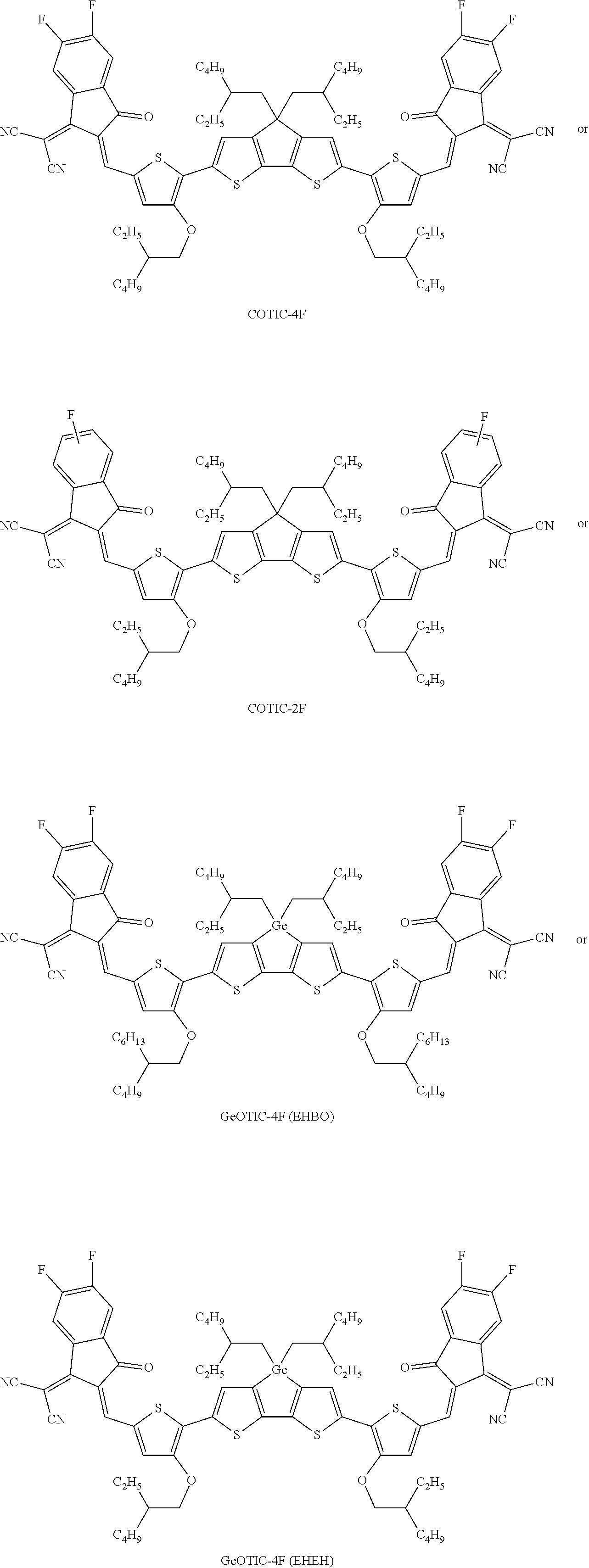

wherein R is a solubilizing chain comprising a substituted or non-substituted alkyl, aryl or alkoxy chain and the side chains comprising C and H may be any solubilizing chain comprising a substituted or non-substituted alkyl, aryl or alkoxy chain.

14. The composition of matter of any of the examples 1-13, wherein the non-fullerene acceptor has the structure:

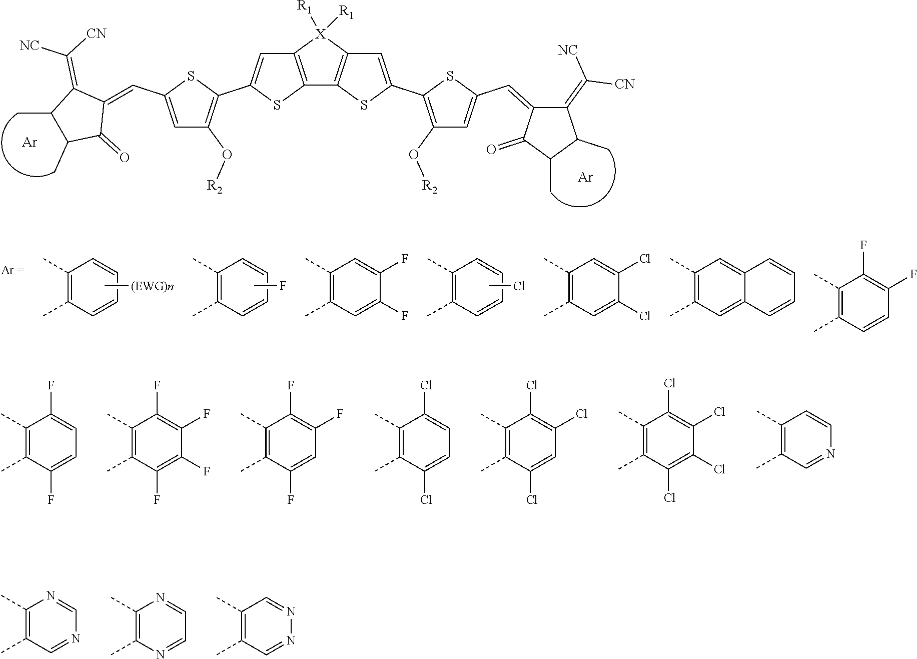

wherein n is an integer; X is C, Si, Ge; EWG=any electron withdrawing group; and each R₁ and R₂ is independently hydrogen or a substituted or non-substituted alkyl, aryl or alkoxy chain.

15. The composition of matter of any of the examples 1-14, wherein the semiconducting compound comprises a semiconducting small molecule having a repeat unit comprising the structure of any of the examples 1-7.

16. The composition of matter of example 15, wherein the semiconducting compound is a small molecule donor comprising the structure of E-A-(D1-A)ₙ-E, wherein:

D1 is

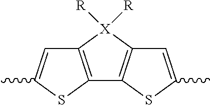

A is

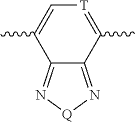

each R is independently hydrogen or a substituted or non-substituted alkyl, aryl, alkoxy or thioether chain;

X is C, Si, Ge, N or P;

each T is independently C—H, N, C—F or C—Cl;

Q is O, S, Se or N—R₄,

E is an alkylated bithiophene, and

n=1, 2 or 3.

17. The composition of matter of any of the examples 1-16, further comprising a bulk heterojunction comprising the composition of matter, wherein the semiconducting compound comprises a donor forming an interconnected network and heterointerface with the non fullerene acceptor, the donor and the acceptor are phase separated, and the donor phase is optionally crystalline. In one or more examples, the NFAs and the donors are combined under conditions to form a blend comprising the donors in first domains and the NFAs in second domains, wherein each of the first domains and second domains are micro or nano sized (e.g., the domains each have a width of 50 nm or less), the first domains and the second domains are sufficiently spatially separated for charge separation of electrons and holes to occur during operation of the solar cell or photodetector (electrons and holes generated in response to incident electromagnetic radiation), but the domains are also sufficiently mixed to form a heterointerface between the donors and the NFAs.

In one or more examples, the first domains and the second domains are not composed of pure donor or pure acceptor phases, respectively, so that the donors and acceptors have sufficient miscibility.

FIG. 17(c) illustrates a bulk heterojunction, showing the first domain (comprising donor, e.g., P2, X2, PM2 or other organic semiconducting compound as described herein) and the second domain (comprising NFA) that are spatially separated but in contact with each other for efficient charge separation. This blend can be the active region in devices as illustrated herein, e.g., as illustrated in FIG. 14a-14b.

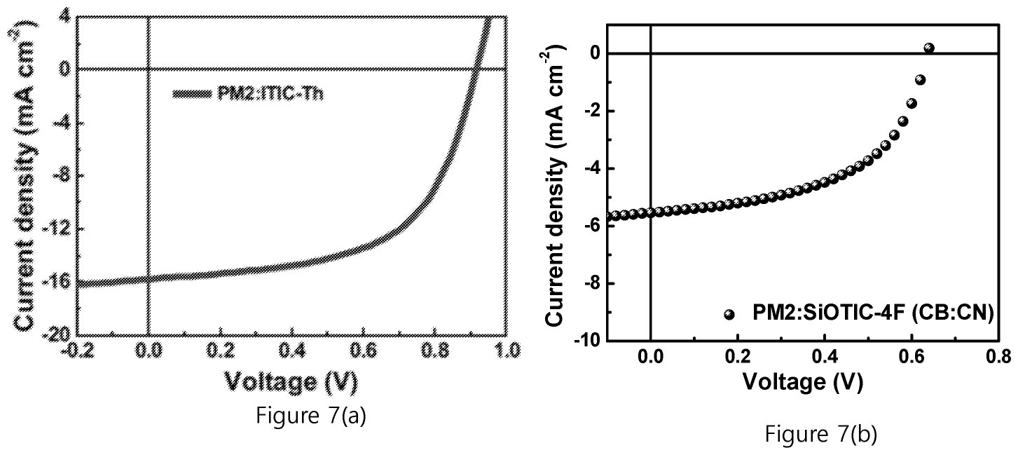

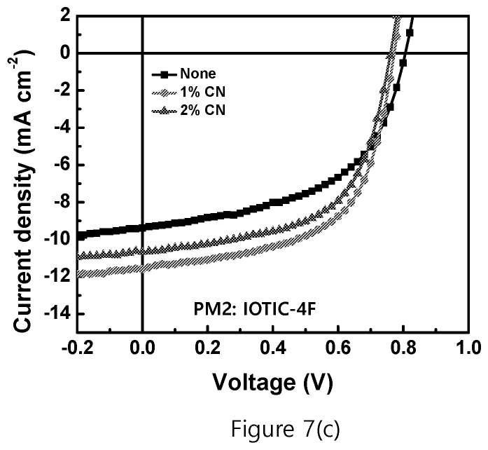

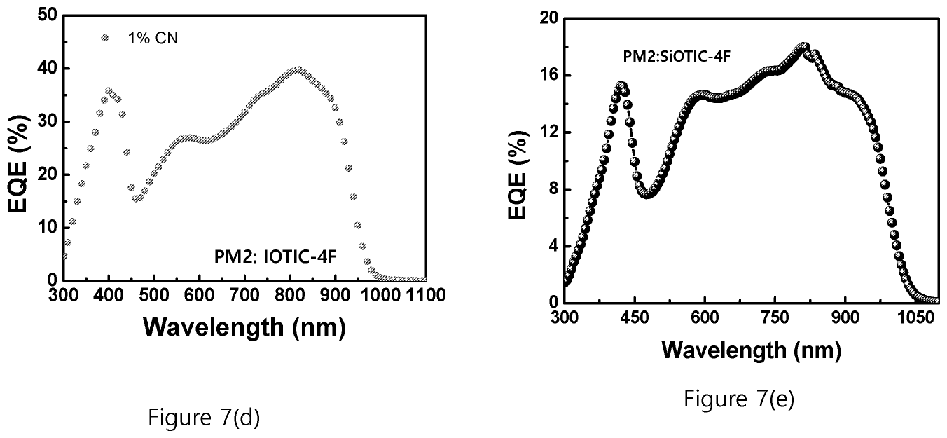

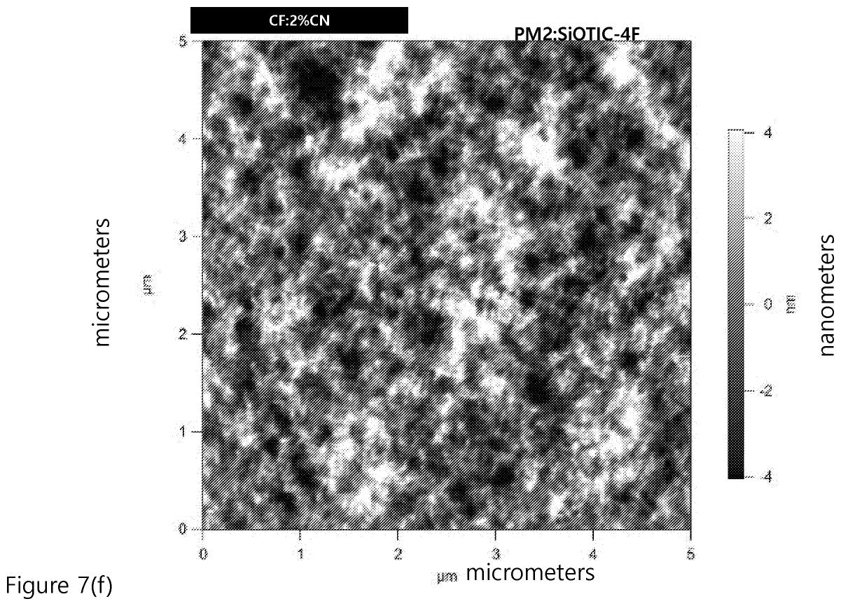

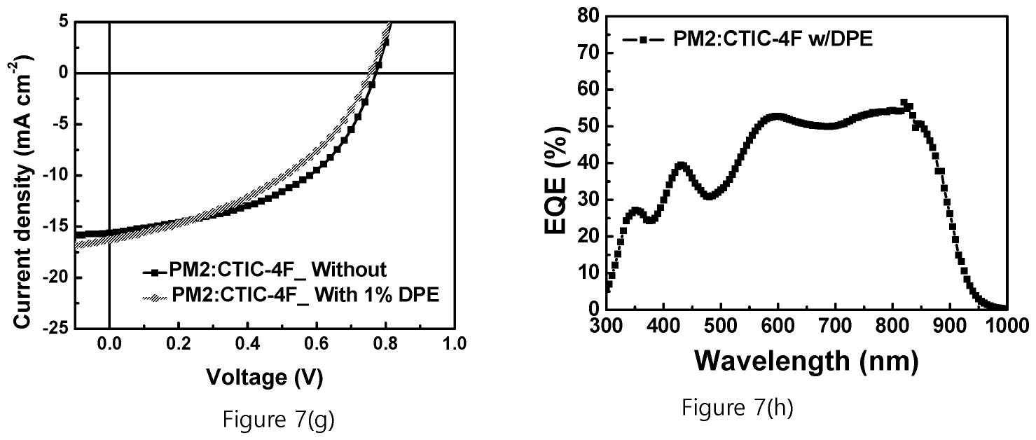

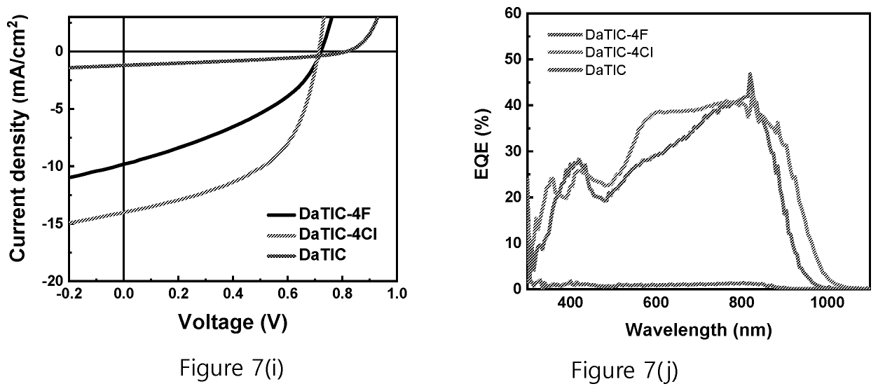

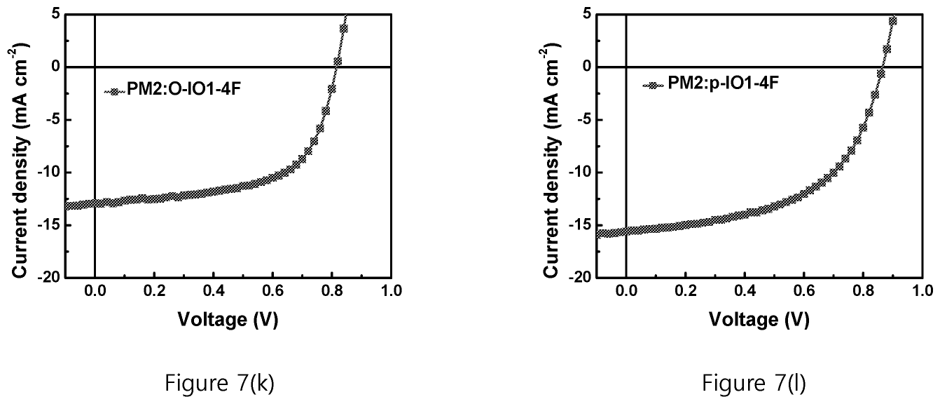

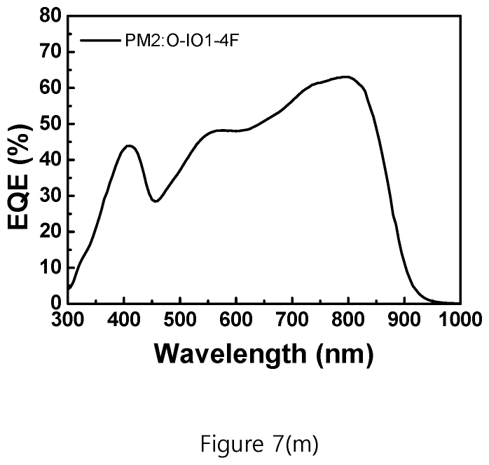

Atomic force microscope images show that is some example blends described herein, the aggregates of donors or acceptor in the blend are relatively small. Conventional devices have suffered from large aggregates of acceptors which easily crystallize using unoptimized molecular structure or film deposition processes. In one or more embodiments described herein, film material structures and compositions and/or processing environment (e.g., solvent, annealing) enable the formation of the donor and acceptor domains that are preferably smaller than 50 nm. FIG. 7(f) shows a good blend that does not have “hills” or height variations of >100 nm. FIG. 7(f) shows an improved morphology because most of the height variation is within the range of −4 to 4 nm (the scale bar). In one or more examples, the surface roughness of the film comprising the blend of donors and acceptors is 4 nm or less over the surface area of the film.

18a. The composition of matter of any of the examples 1-17, wherein the non fullerene acceptor has a bandgap of 1.3 eV or less (e.g., in a range of 0.8 eV-1.3 eV or 1 eV-1.3 eV where eV is electron volts).

19. A device comprising the composition of matter of any of the examples 1-18, wherein the device comprises a solar cell.

20. A device comprising the composition of matter of any of the examples 1-19, wherein the device comprises a photodetector.

21. The device of any of the examples 19-20, further comprising an active region comprising the composition of matter of any of the examples 1-18, wherein holes and electrons are generated in the active region in response to electromagnetic radiation incident on the active region,

the electrons are collected in the non-fullerene acceptor and are transmitted through to a cathode,

the holes are collected in the semiconducting compound comprising a donor and transmitted through to an anode,

so that the device outputs current in response to the electromagnetic radiation.

22. The device of any of the examples 19-21, comprising:

a film comprising a thickness of less than 1 micrometer and comprising:
the semiconducting compound having a transmittance of at least 70% for visible electromagnetic radiation having the wavelength in a range of 400 nm to 600 nm, and
the photodetector having an external quantum efficiency (EQE) of at least 30%, a responsivity of at least 0.1 A/W, and a specific detectivity of at least 10¹⁰ Jones for the electromagnetic radiation having the wavelength in a range of 700 nm-900 nm.

23. The device of example 22, wherein the composition of matter has the structure:

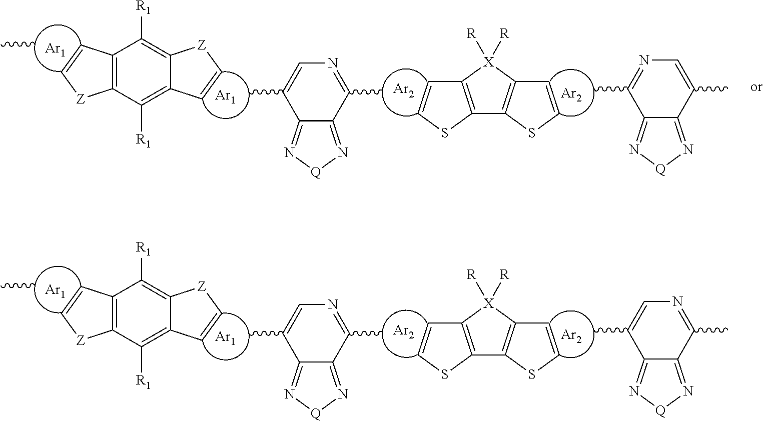

each Ar₁ and Ar₂ is independently a substituted or non-substituted aromatic functional group, or each Ar₁ and Ar₂ is nothing and the valence of the ring is completed with hydrogen.

each Z is independently O, S, Se, or N—R₄;

each X is C, Si, Ge, N or P;

each Q is independently O, S Se, or N—R₄;

and

each R, R₁ and R₄ is independently hydrogen or a substituted or non-substituted alkyl, aryl, alkoxy or thioether chain.

24. The device of example 23, wherein the semiconducting compound has the structure:

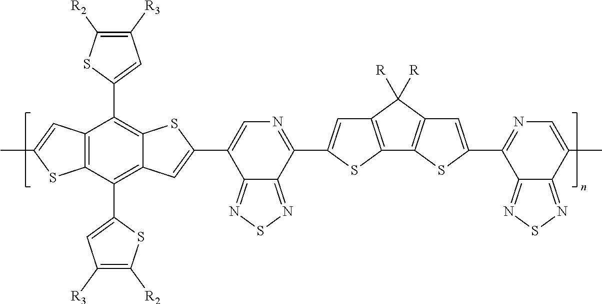

where each R, R₂ and R₃ is independently hydrogen or a substituted or non-substituted alkyl, aryl, alkoxy or thioether chain, and

n is an integer.

25. The device of example 22, further comprising:

a source of the visible electromagnetic radiation;

the photodetector on the source; and

a cover or window on the photodetector, such that the visible electromagnetic radiation is transmitted through the photodetector and the cover or window to a viewer.

26. The device of any of the examples 20-25, further comprising a biomedical sensor, wherein the photodetector measures electromagnetic radiation scattered or reflected from living tissue or cells.

27. A device, comprising:

an active region comprising organic semiconducting compounds (e.g., of any of the examples 1-18) outputting an electrical signal in response to electromagnetic radiation incident on the active region, the active region having:

a thickness less than 1 micrometer;

a transmittivity of at least 70% for the electromagnetic radiation having the wavelength in a range of 400 nanometers (nm) to 600 nm, and

an EQE of at least 30% for the electromagnetic radiation having the wavelength in a range of 700 nm-900 nm.

28. The device of example 27, wherein the semiconducting compound has the structure:

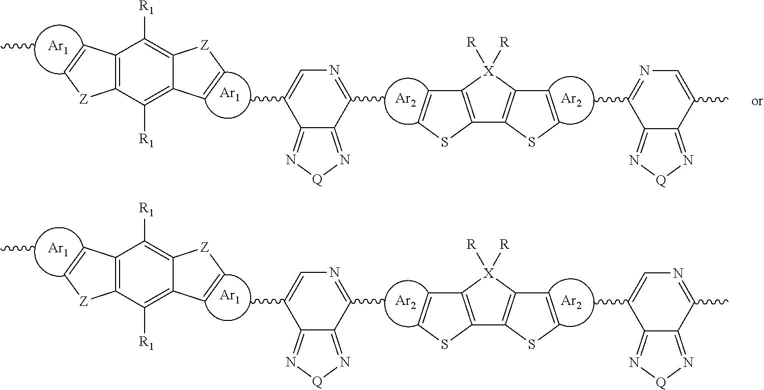

each Ar₁ and Ar₂ is independently a substituted or non-substituted aromatic functional group, or each Ar₁ and Ar₂ is nothing and the valence of the ring is completed with hydrogen.

each Z is independently O, S, Se, or N—R₄;

each X is C, Si, Ge, N or P;

each Q is independently O, S Se, or N—R₄,

and

each R, R₁ and R₄ is independently hydrogen or a substituted or non-substituted alkyl, aryl, alkoxy or thioether chain.

29. An infrared photodetector, comprising:

a first electrode;

a first carrier transport layer;

an active layer, wherein the first carrier transport layer is between the first electrode and the active layer;

a second carrier transport layer, wherein the active layer is between the first carrier transport layer and the second carrier transport layer; and

a second electrode on the second carrier transport layer, wherein:

the active layer comprises the composition of matter of any of the examples 1-18, and

a transmittance of the photodetector is 50% or more at the wavelengths of 400-600 nm.

30. The photodetector of example 29, wherein the semiconducting compound in the active layer comprises P2.

31. The photodetector of example 29, wherein the semiconducting compound in the active layer comprises PM2.

32. A device comprising the photodetector of any of the examples 29-31, comprising:

a display emitting the wavelengths;

the photodetector on or above the display;

a screen on or above the photodetector, wherein the display is readable by eye of a viewer through the photodetector; and

a circuit connected to the photodetector, the circuit determining a gesture of the viewer from a signal outputted from photodetector in response to infrared radiation incident on the photodetector.

33. The device of example 32, wherein the photodetector has a thickness of 3 micrometers or less.

### REFERENCES FOR FIRST EXAMPLE

The following references are incorporated by reference herein
1. Yang (Michael) Yang, Wei Chen, Letian Dou, Wei-Hsuan Chang, Hsin-Sheng Duan, Brion Bob, Gang Li and Yang, Nature Photonics, 2015, 9, 190-198
2. Seyeong Song, Kang Taek Lee, Chang Woo Koh, Hyebeom Shin, Mei Gao, Han Young Woo, Doojin Vak and Jin Young Kim, Energy Environ. Sci., 2018, 11, 3248-3255
3. Ram Prakash Singh, Omkar Singh Kushwaha, Macromol. Symp. 2013, 327, 128-149
4. P. R. Berger and M. Kim, J. Renewable Sustainable Energy, 2018, 10, 013508
5. Yankang Yang, Zhi-Guo Zhang, Haijun Bin, Shanshan Chen, Liang Gao, Lingwei Xue, Changduk Yang, and Yongfang Li, J. Am. Chem. Soc. 2016, 138, 15011-15018
6. Yuze Lin, Jiayu Wang, Zhi-Guo Zhang, Huitao Bai, Yongfang Li, Daoben Zhu, and Xiaowei Zhan, Adv Mater. 2015, 27, 1170-1174
7. Xin Song, Nicola Gasparini, Long Ye, Huifeng Yao, Jianhui Hou, Harald Ade, and Derya Baran, ACS Energy Lett. 2018, 3, 669-676
8. Jianqiu Wang, Shenkun Xie, Dongyang Zhang, Rong Wang, Zhong Zheng, Huiqiong Zhou and Yuan Zhang, J. Mater. Chem. A, 2018, 6, 19934-19940
9. Sixing Xiong, Lin Hu, Lu Hu, Lulu Sun, Fei Qin, Xianjie Liu, Mats Fahlman, and Yinhua Zhou, Adv. Mater. 2019, 31, 1806616
10. Lingling Zhan, Shuixing Li, Huotian Zhang, Feng Gao, Tsz-Ki Lau, Xinhui Lu, Danyang Sun, Peng Wang, Minmin Shi,* Chang-Zhi Li, and Hongzheng Chen, Adv. Sci. 2018, 5, 1800755
11. Weiwei Li, Koen H. Hendriks, W. S. Christian Roelofs, Youngju Kim, Martijn M. Wienk, and René A. J. Janssen, Adv. Mater. 2013, 25, 3182-3186
12. Letian Dou, Chun-Chao Chen, Ken Yoshimura, Kenichiro Ohya, Wei-Hsuan Chang, Jing Gao, Yongsheng Liu, Eric Richard and Yang, Macromolecules, 2013, 46, 3384-3390
13. Zhong Zheng, Shaoqing Zhang, Jianqi Zhang, Yunpeng Qin, Wanning Li, Runnan Yu, Zhixiang Wei, and Jianhui Hou, Adv. Mater. 2016, 28, 5133-5138
14. Xiaofen Chen, Xiaofeng Liu, Mark A. Burgers, Ye Huang, and Guillermo C. Bazan, Angew. Chem. Int. Ed. 2014, 53, 14378-14381
15. Xiaofeng Liu, Yanming Sun, Louis A. Perez, Wen, Michael F. Toney, Alan J. Heeger, and Guillermo C. Bazan, J. Am. Chem. Soc. 2012, 134, 20609-20612
16. Xiaofeng Liu, Yanming Sun, Ben B. Y. Hsu, Andreas Lorbach, Li Qi, Alan J. Heeger, and Guillermo C. Bazan, J. Am. Chem. Soc. 2014, 136, 5697-5708
17. Xiaofeng Liu, Ben B. Y. Hsu, Yanming Sun, Cheng-Kang Mai, Alan J. Heeger, and Guillermo C. Bazan, J. Am. Chem. Soc. 2014, 136, 16144-16147
18. Christopher J. Takacs, Yanming Sun, Gregory C. Welch, Louis A. Perez, Xiaofeng Liu, Wen, Guillermo C. Bazan, and Alan J. Heeger, J. Am. Chem. Soc. 2012, 134, 16597-16606
19. Ming Wang, Hengbin Wang, Michael Ford, Jianyu Yuan, Cheng-Kang Mai, Stephanie Fronk and Guillermo C. Bazan, J. Mater. Chem. A, 2016, 4, 15232-15239
20. Ye Huang, Edward J. Kramer, Alan J. Heeger, and Guillermo C. Bazan, Chem. Rev. 2014, 114, 7006-7043
21. Hansol Lee, Chaneui Park, Dong Hun Sin, Jong Hwan Park, and Kilwon Cho, Adv. Mater. 2018, 30, 1800453
22. Jaewon Lee, Seo-Jin Ko, Hansol Lee, Jianfei Huang, Ziyue Zhu, Martin Seifrid, Joachim Vollbrecht, Viktor V. Brus, Akchheta Karki, Hengbin Wang, Kilwon Cho, Thuc-Quyen Nguyen, and Guillermo C. Bazan, ACS Energy Lett. 2019, 4, 1401-1409
23. Liyan Yang, Shaoqing Zhang, Chang He, Jianqi Zhang, Huifeng Yao, Yang, Yun Zhang, Wenchao Zhao, and Jianhui Hou, J. Am. Chem. Soc. 2017, 139, 1958-1966
24. Haijun Bin, Yankang Yang, Zhi-Guo Zhang, Long Ye, Masoud Ghasemi, Shanshan Chen, Yindong Zhang, Chunfeng Zhang, Chenkai Sun, Lingwei Xue, Changduk Yang, Harald Ade, and Yongfang Li, J. Am. Chem. Soc. 2017, 139, 5085-5094
25. Yong Huo, Xiao-Ting Gong, Tsz-Ki Lau, Tong Xiao, Cenqi Yan, Xinhui Lu, Guanghao Lu, Xiaowei Zhan, and Hao-Li Zhang, Chem. Mater. 2018, 30, 8661-8668
26. Ke Gao, Sae Byeok Jo, Xueliang Shi, Li Nian, Ming Zhang, Yuanyuan Kan, Francis Lin, Bin Kan, Bo Xu, Qikun Rong, Lingling Shui, Feng Liu, Xiaobin Peng, Guofu Zhou, Yong Cao, and Alex K.-Y. Jen, Adv. Mater. 2019, 31, 1807842
27. Maged Abdelsamie, Neil D. Treat, Kui Zhao, Caitlin McDowell, Mark A. Burgers, Ruipeng Li, Detlef-M. Smilgies, Natalie Stingelin, Guillermo C. Bazan, and Aram Amassian, Adv. Mater. 2015, 27, 7285-7292
28. (a) M. A. Green, K. Emery, Y. Hishikawa, W. Warta, E. D. Dunlop, Progress Photoyoh: Res Appl, 2015, 23, 1; (b) N.
G. Park, M. Gratzel, T. Miyasaka, K. Zhu, K. Emery, Nat Energy, 2016, 1, 16152.
29. C. J. M. Emmott, J. A. R\ddot{ }ohr, M. Campoy-Quiles, T. Kirchartz, A. Urbina, N. J. Ekins-Daukesa, J. Nelson, Energy Environ. Sci., 2015, 8, 1317.
30. W. Chen, Q. Zhang, J. Mater. Chem C, 2017, 5, 1275.
31. Y. Lin, J. Wang, Z.-G. Zhang, H. Bai, Y. Li, D. Zhu, X. Zhan, Adv. Mater., 2015, 27, 1170.
32. (a) W. Zhao, S. Li, H. Yao, S. Zhang, Y. Zhang, B. Yang, J. Hou, J. Am. Chem. Soc., 2017, 139, 7148; (b) D. Baran, T. Kirchartz, S. Wheeler, S. Dimitrov, M. Abdelsamie, J. Gorman, R. S. Ashraf, S. Holliday, A. Wadsworth, N. Gasparini, P. Kaienburg, H. Yan, A.
33. Design of Nonfullerene Acceptors with Near-Infrared Light Absorption Capabilities. Advanced Energy Materials 2018, online. DOI: 10.1002/aenm.201801209.
34. Influence of molecular structure on the performance of low Voe loss polymer solar cells, Journal of Materials Chemistry A, 2016, 4, 15232.
35. Design and Properties of Intermediate-Sized Narrow Band-Gap Conjugated Molecules Relevant to Solution-Processed Organic Solar Cells, Journal of the American Chemical Society, 136, 5607-5708.
36. Single-Junction Organic Solar Cell with over 15% Efficiency Using Fused-Ring Acceptor with Electron-Deficient Core, Joule, 2019, 4, 1140.
37. X. Liu, Y. Lin, Y. Liao, J. Wu, Y. Zheng, J. Mater. Chem. C 2018, 6, 3499.
38. Z. Wu, W. Yao, A. E. London, J. D. Azoulay, T. N. Ng, Adv. Funct. Mater. 2018, 28, 1800391.
39. Q. Lin, A. Armin, P. L. Burn, P. Meredith, Laser Photonics Rev. 2016, 10, 1047.
40. L. Meng, Y. Zhang, X. Wan, C. Li, X. Zhang, Y. Wang, X. Ke, Z. Xiao, L. Ding, R Xia, H. Yip, Y. Cao, Y. Chen, Science 2018, 1094-1098.
41. Y. Lin, Y. Firdaus, M. Nugraha, F. Liu, S. Karuthedath, A. Emwas, W. Zhang, A. Seitkhan, M. Neophytou, H. Faber, E. Yengel, I. McCulloch, L. Tsetseris, F. Laquai, T. Anthopoulos, Adv. Sci. 2020, 7, 1903419.

### REFERENCES FOR SECOND EXAMPLE

The following references are incorporated by reference herein.
1. Lei Ying, Ben B. Y. Hsu, Hongmei Zhan, Gregory C. Welch, Peter Zalar, Louis A. Perez, Edward J. Kramer, Thuc-Quyen Nguyen, Alan J. Heeger, Wai-Yeung Wong, and Guillermo C. Bazan, J. Am. Chem. Soc. 2011, 133, 18538-1854
2. P. R Berger and M. Kim, J. Renewable Sustainable Energy, 2018, 10, 013508
3. Yankang Yang, Zhi-Guo Zhang, Haijun Bin, Shanshan Chen, Liang Gao, Lingwei Xue, Changduk Yang, and Yongfang Li, J. Am. Chem. Soc. 2016, 138, 15011-15018
4. Yuze Lin, Jiayu Wang, Zhi-Guo Zhang, Huitao Bai, Yongfang Li, Daoben Zhu, and Xiaowei Zhan, Adv. Mater. 2015, 27, 1170-1174
5. Yang (Michael) Yang, Wei Chen, Letian Dou, Wei-Hsuan Chang, Hsin-Sheng Duan, Brion Bob, Gang Li and Yang, Nature Photonics, 2015, 9, 190-198
6. Seyeong Song, Kang Taek Lee, Chang Woo Koh, Hyebeom Shin, Mei Gao, Han Young Woo, Doojin Vak and Jin Young Kim, Energy Environ. Sci., 2018, 11, 3248-3255
7. Ram Prakash Singh, Omkar Singh Kushwaha, Macromol. Symp. 2013, 327, 128-149.
8. Xin Song, Nicola Gasparini, Long Ye, Huifeng Yao, Jianhui Hou, Harald Ade, and Derya Baran, ACS Energy Lett. 2018, 3, 669-676
9. Jianqiu Wang, Shenkun Xie, Dongyang Zhang, Rong Wang, Zhong Zheng, Huiqiong Zhou and Yuan Zhang, J. Mater. Chem. A, 2018, 6, 19934-19940
10. Sixing Xiong, Lin Hu, Lu Hu, Lulu Sun, Fei Qin, Xianjie Liu, Mats Fahlman, and Yinhua Zhou, Adv. Mater. 2019, 31, 1806616
11. Lingling Zhan, Shuixing Li, Huotian Zhang, Feng Gao, Tsz-Ki Lau, Xinhui Lu, Danyang Sun, Peng Wang, Minmin Shi,* Chang-Zhi Li, and Hongzheng Chen, Adv. Sci. 2018, 5, 1800755
12. Weiwei Li, Koen H. Hendriks, W. S. Christian Roelofs, Youngju Kim, Martijn M. Wienk, and René A. J. Janssen, Adv. Mater. 2013, 25, 3182-3186
13. Letian Dou, Chun-Chao Chen, Ken Yoshimura, Kenichiro Ohya, Wei-Hsuan Chang, Jing Gao, Yongsheng Liu, Eric Richard and Yang, Macromolecules, 2013, 46, 3384-3390
14. Zhong Zheng, Shaoqing Zhang, Jianqi Zhang, Yunpeng Qin, Wanning Li, Runnan Yu, Zhixiang Wei, and Jianhui Hou, Adv. Mater. 2016, 28, 5133-5138
15. Liyan Yang, Shaoqing Zhang, Chang He, Jianqi Zhang, Huifeng Yao, Yang, Yun Zhang, Wenchao Zhao, and Jianhui Hou, J. Am. Chem. Soc. 2017, 139, 1958-1966
16. Haijun Bin, Yankang Yang, Zhi-Guo Zhang, Long Ye, Masoud Ghasemi, Shanshan Chen, Yindong Zhang, Chunfeng Zhang, Chenkai Sun, Lingwei Xue, Changduk Yang, Harald Ade, and Yongfang Li, J. Am. Chem. Soc. 2017, 139, 5085-5094
17. Yong Huo, Xiao-Ting Gong, Tsz-Ki Lau, Tong Xiao, Cenqi Yan, Xinhui Lu, Guanghao Lu, Xiaowei Zhan, and Hao-Li Zhang, Chem. Mater. 2018, 30, 8661-8668
18. Ke Gao, Sae Byeok Jo, Xueliang Shi, Li Nian, Ming Zhang, Yuanyuan Kan, Francis Lin, Bin Kan, Bo Xu, Qikun Rong, Lingling Shui, Feng Liu, Xiaobin Peng, Guofu Zhou, Yong Cao, and Alex K.-Y. Jen, Adv. Mater. 2019, 31, 1807842
19. Design of Nonfullerene Acceptors with Near-Infrared Light Absorption Capabilities. Advanced Energy Materials 2018, 8, 1801209.
20. Influence of molecular structure on the performance of low Voc loss polymer solar cells, Journal of Materials Chemistry A, 2016, 4, 15232.
21. Design and Properties of Intermediate-Sized Narrow Band-Gap Conjugated Molecules Relevant to Solution-Processed Organic Solar Cells, Journal of the American Chemical Society. 2014, 136, 5697-5708.

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

1. A composition of matter, comprising:
a semiconducting compound of the structure (and isomers thereof): 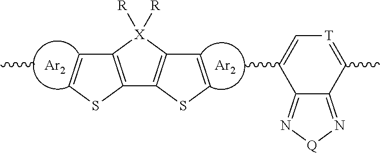
wherein:
each Ar₂ is independently a substituted or non-substituted aromatic functional group, or Ar₂ is nothing and the valence of the ring is completed with hydrogen;
each Q is independently O, S Se, or N—R₄;
each R and R₄ is independently hydrogen or a substituted or non-substituted alkyl, aryl, alkoxy or thioether chain;
T is N, C—F, or C—Cl;
X is C, Si, Ge, N or P; and
a non-fullerene acceptor combined with the semiconducting compound.

2. The composition of matter of claim 1, wherein the semiconducting compound comprises the structure:

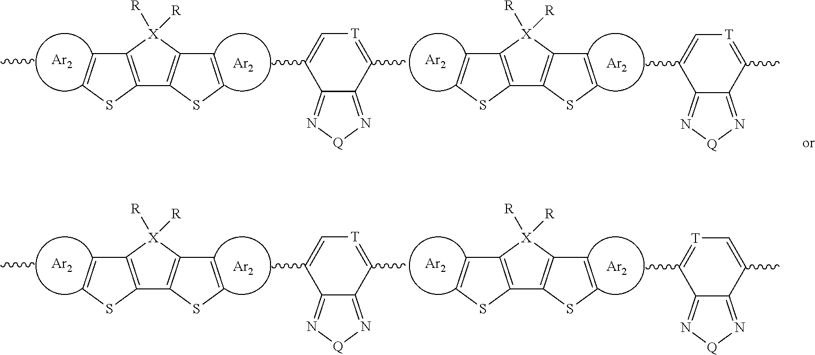

3. The composition of matter of claim 2, wherein the semiconducting compound comprises the structure:

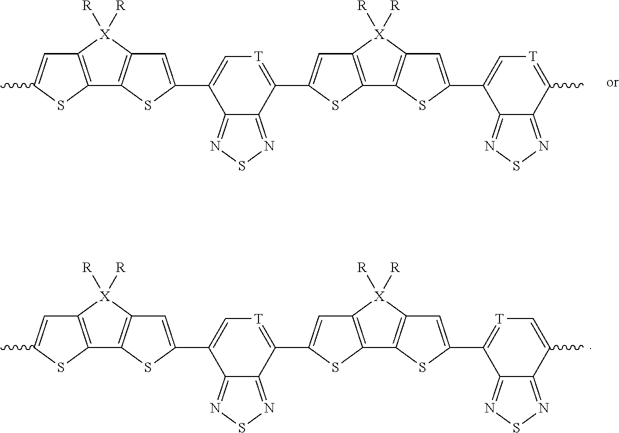

4. The composition of matter of claim 1, wherein the semiconducting compound comprises the structure: 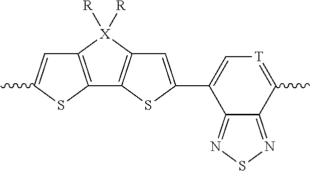
wherein:
each R is independently hydrogen or a substituted or non-substituted alkyl, aryl, alkoxy or thioether chain;
T is N, C—F, or C—Cl; and
X is C, Si, Ge, N or P.

5. The composition of matter of claim 1, wherein the semiconducting compound further comprises the structure: 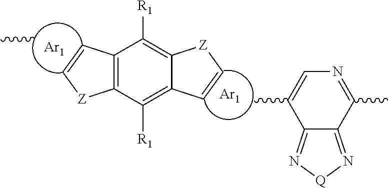
wherein:
each Ar₁ is independently a substituted or non-substituted aromatic functional group, or each Ar₁ is nothing and the valence of the ring is completed with hydrogen.
each Z is independently O, S, Se, or N—R₄;
each R₁, R₄ is independently hydrogen or a substituted or non-substituted alkyl, aryl, alkoxy or thioether chain.
each Q is independently O, S Se, or N—R₄;

6. The composition of matter of claim 5, wherein the semiconducting compound comprises the structure:

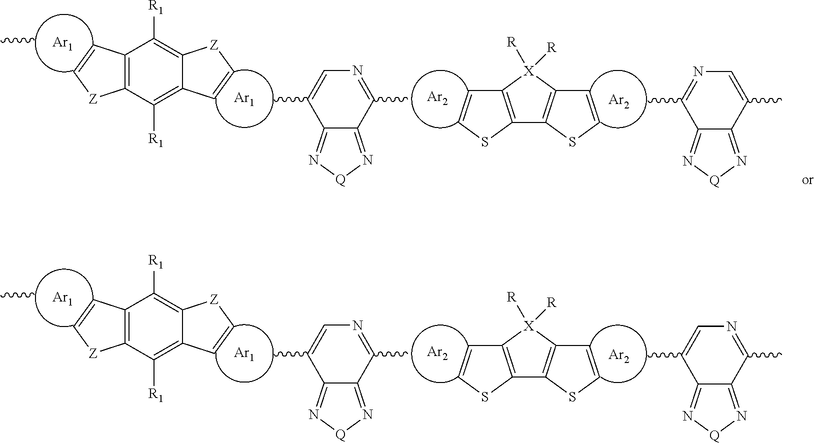

7. The composition of matter of claim 6, wherein the semiconducting compound comprises the structure:

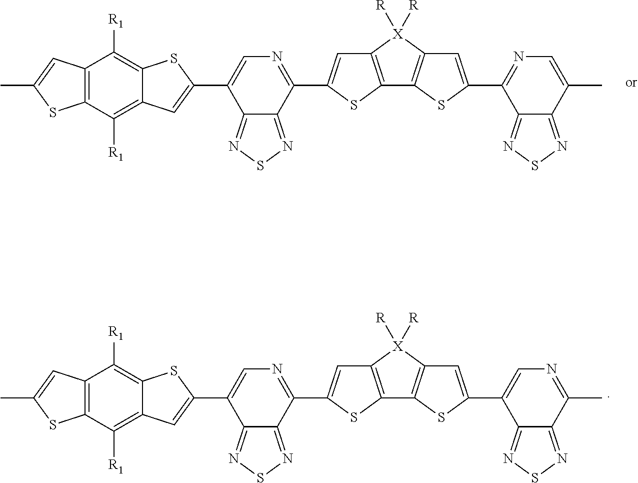

8. The composition of matter of claim 1, wherein the non-fullerene acceptor has the structure: 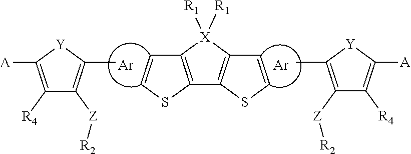
wherein:
each Ar is independently a substituted or non-substituted aromatic functional group, or each Ar is independently nothing and the valence of its respective thiophene ring is completed with hydrogen;
X is C, Si, Ge, N or P; Y is O, S, Se or N—R₃;
Z is O, S, Se, or N—R₃;
A is an acceptor moiety; and
each R₁, R₂ and R₃ is independently hydrogen or a substituted or non-substituted alkyl, aryl or alkoxy chain; R₄ is either a hydrogen or the same as Z—R₂.

9. The composition of matter of claim 8, wherein the non-fullerene acceptor has a bandgap less than or equal to the bandgap of the semiconducting compound.

10. The composition of matter of claim 9, wherein the semiconducting compound comprises a semiconducting polymer having a repeat unit comprising the structure of any of the claims 1-7.

11. The composition of matter of claim 10, wherein the semiconducting polymer comprises a semiconducting polymer having the structure:

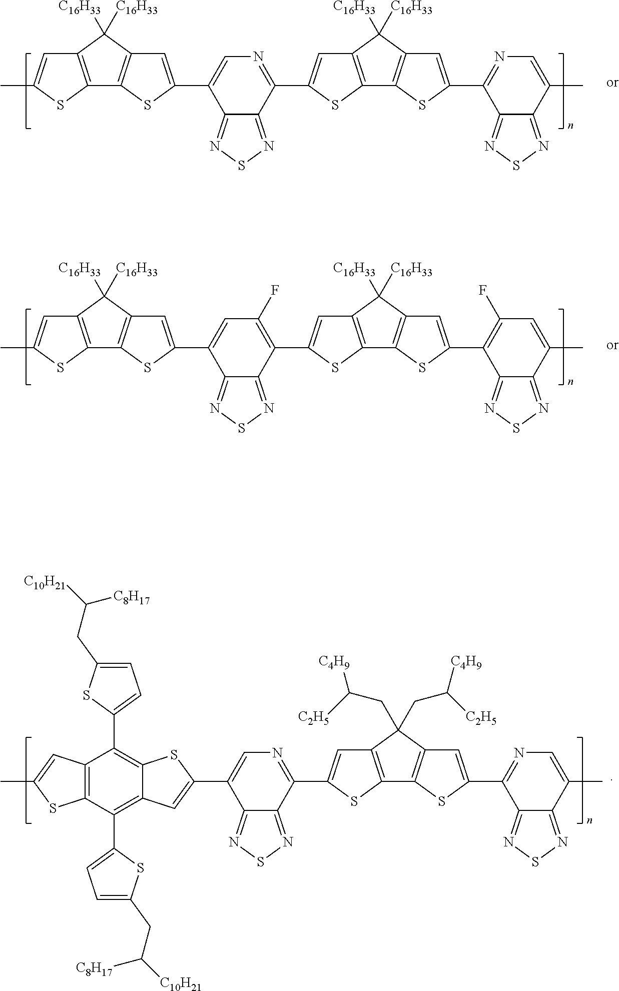

12. The composition of matter of claim 10, wherein the acceptor unit in the semiconducting compound is regioregularly arranged along the conjugated main chain section.

13. The composition of matter of claim 1, wherein the non-fullerene acceptor comprises: 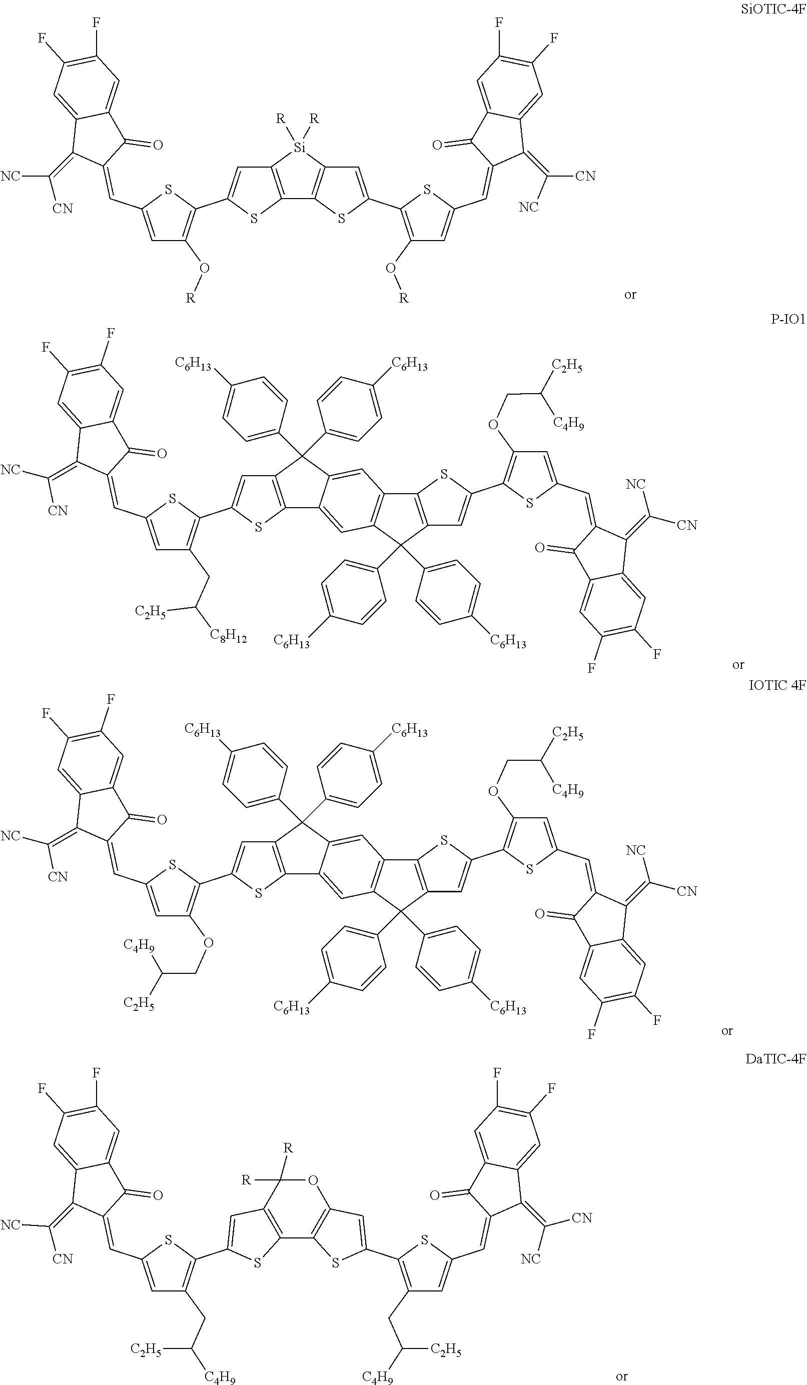 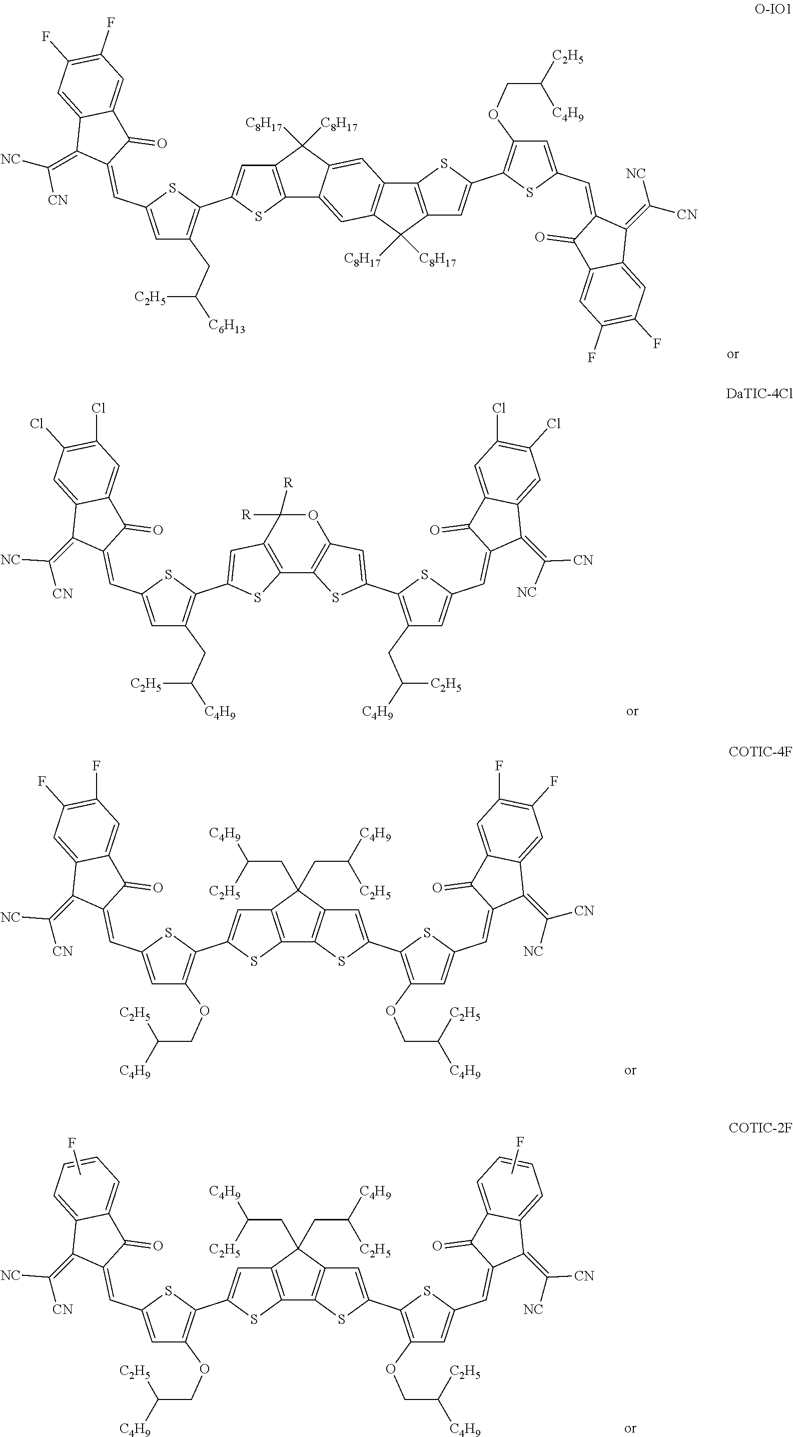 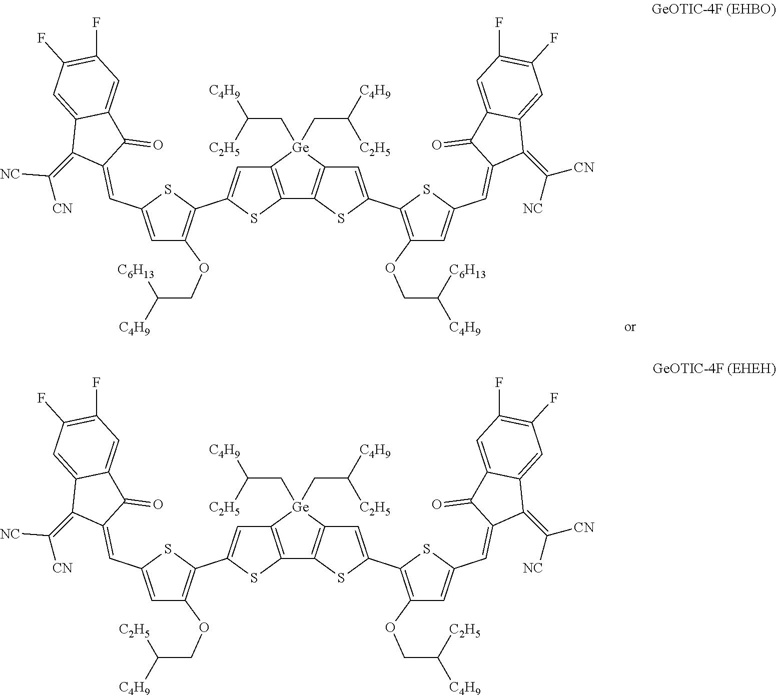
wherein R is a solubilizing chain comprising a substituted or non-substituted alkyl, aryl or alkoxy chain and the side chains comprising C and H may be any solubilizing chain comprising a substituted or non-substituted alkyl, aryl or alkoxy chain.

14. The composition of matter of claim 1, wherein the non-fullerene acceptor has the structure: 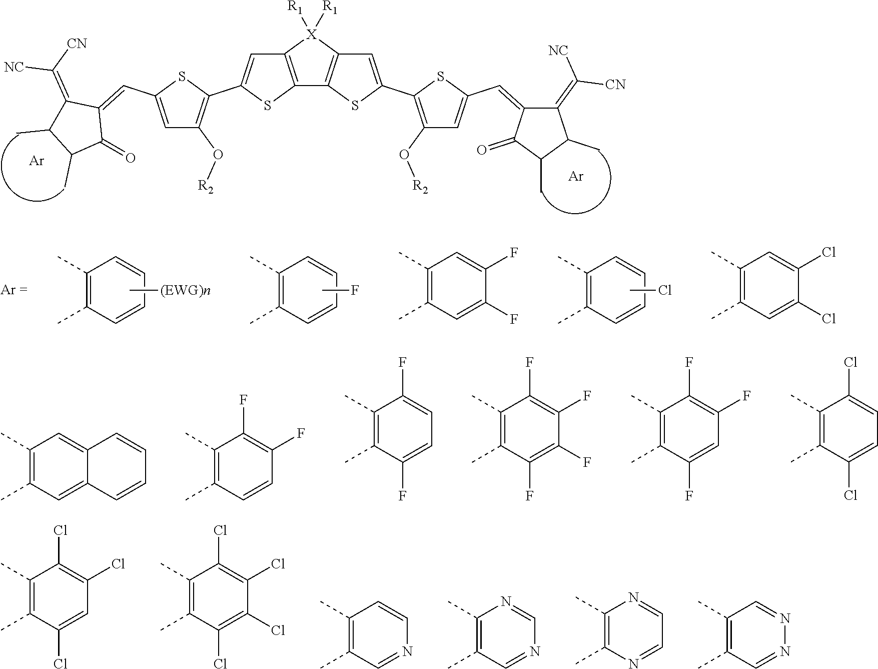
wherein n is an integer; X is C, Si, Ge; EWG=any electron withdrawing group; and each R₁ and R₂ is independently hydrogen or a substituted or non-substituted alkyl, aryl or alkoxy chain.

15. The composition of matter of claim 9, wherein the semiconducting compound comprises a semiconducting small molecule having a repeat unit comprising the structure of claim 1-7.

16. The composition of matter of claim 15, wherein the semiconducting compound is a small molecule donor comprising the structure of E-A-(D1-A)ₙ-E, wherein:
D1 is 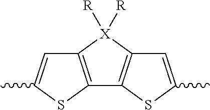
A is 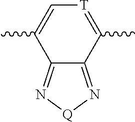
each R is independently hydrogen or a substituted or non-substituted alkyl, aryl, alkoxy or thioether chain;
X is C, Si, Ge, N or P;
each T is independently C—H, N, C—F or C—Cl;
Q is O, S, Se or N—R₄,
E is an alkylated bithiophene, and
n=1, 2 or 3.

17. The composition of matter of claim 1, further comprising a bulk heterojunction comprising the composition of matter, wherein the semiconducting compound comprises a donor forming an interconnected network with the non fullerene acceptor, the donor and the acceptor are phase separated, and the donor phase is crystalline.

18. The composition of matter of claim 1, wherein the non fullerene acceptor has a bandgap of 1.3 eV or less.

19. A device comprising the composition of matter of claim 1, wherein the device comprises a solar cell, further comprising an active region comprising the composition of matter, wherein holes and electrons are generated in the active region in response to electromagnetic radiation incident on the active region.
the electrons are collected in the non-fullerene acceptor and are transmitted through to a cathode,
the holes are collected in the semiconducting compound comprising a donor and transmitted through to an anode,
so that the device outputs current in response to the electromagnetic radiation.

20. A device comprising the composition of matter of claim 1, wherein the device comprises a photodetector, further comprising an active region comprising the composition of matter, wherein holes and electrons are generated in the active region in response to electromagnetic radiation incident on the active region,
the electrons are collected in the non-fullerene acceptor and are transmitted through to a cathode,
the holes are collected in the semiconducting compound comprising a donor and transmitted through to an anode,
so that the device outputs current in response to the electromagnetic radiation.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)
